(19) United States
(12) Patent Application Publication
(10) Pub. No.: US 2025/0326736 A1
(43) Pub. Date: Oct. 23, 2025

(54) FLUORESCENTLY-ACTIVE FREE RADICAL TAGS FOR SIMULTANEOUS GLYCAN QUANTITATION AND CHARACTERIZATION

(71) Applicant: MONTCLAIR STATE UNIVERSITY, Montclair, NJ (US)

(72) Inventors: Jinshan GAO, Montclair, NJ (US); Rayan MURTADA, Clifton, NJ (US)

(21) Appl. No.: 18/870,680

(22) PCT Filed: Jun. 2, 2023
(86) PCT No.: PCT/US2023/024228

§ 371 Date: Dec. 1, 2024

Related U.S. Application Data
(60) Provisional application No. 63/348,795, filed on Jun. 3, 2022.

(51) Int. Cl. C07D 401/12; C07D 211/94; G01N 33/58

(52) U.S. Cl. CPC C07D 401/12; C07D 211/94; G01N 33/582; G01N 2400/10

(57) ABSTRACT

Provided are compounds useful for the quantitation and/or characterization of glycans and other similar molecules. Such compounds include compounds of formula (1a) or (1b) substituted with at least a TEMPO group or analog thereof and a coupling site wherein X¹, X² and X may be one of NH, N-alkyl, O, NO, C═O, CH, N or CR⁹.

### CROSS-REFERENCE TO RELATED APPLICATIONS

This application claims priority of U.S. Provisional Application No. 63/348,795, filed Jun. 3, 2022, the contents of which is hereby incorporated by reference in its entirety.

### FIELD OF THE INVENTION

The invention is directed to fluorescently-active free radical tags including to substituted compounds of Formula (Ia) and Formula (Ib):

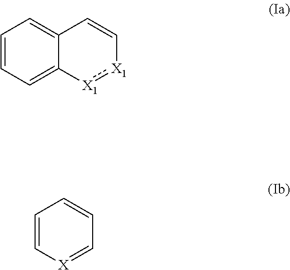

and to methods of using these and related compounds. One such use is to quantitate and characterize glycans.

All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

The study of glycans and related structures, or glycomics, has provoked significant thought and research in recent decades. Much attention has shifted towards the ability of quantitating, distinguishing, and characterizing glycans and their isomers. Unlike other biopolymer molecules, such as peptides and nucleic acids, which involve the linkage of subunits via a defined backbone that is consisted of amide and phosphodiester bonds, respectively, glycans can have monosaccharide subunits arranged in a branched manner via complex regiochemical and stereochemical linkages. Because glycan structures differ and are described by their types of connectivity, monosaccharide composition, and overall configuration, many constitutional isomers and stereoisomers are possible. Unlike other biomolecules, glycans are involved in essential functions, such as major metabolic, signaling, and structural roles, within living systems. Moreover, glycans are known to vary in structure and quantity upon the onset of many diseases, including but not limited to cancer metastases, autoimmune diseases, hereditary diseases, pathogen-host interactions, immune recognition, and Alzheimer's. Therefore, further advancements in the field of glycomics may lead to the future use of glycans as early biomarkers for certain human diseases in the medical setting.

Also important is the analysis of glycosylations derived from biopharmaceuticals which require an analytical approach that facilitates the characterization of glycans for monitoring and regulation purposes. The glycosylation of biopharmaceuticals is difficult to control as slight condition differences in pH, cell line, dissolved oxygen, temperature, ammonia concentrations, and manufacturing mode can influence glycosylation. As a result, a glycan characterization technique is required since the presence of certain glycosylations can affect the safety, efficacy, half-life, immune response, and binding. For example, the monoclonal antibody drug cetuximab, which targets the epidermal growth factor receptor to inhibit the progression of colorectal cancer, was processed with the presence of gal-α(1→3)-gal which caused anaphylaxis in patients. Ultimately, the rapid development of an improved and modern analytical technique that is capable of both quantitating and characterizing glycans from a host of sources across numerous industries is significantly beneficial.

Numerous techniques have been involved in the study of glycans, including high-performance liquid chromatography (HPLC), ion mobility, electrophoresis, and nuclear magnetic resonance (NMR) spectroscopy. For glycan structural analysis, HPLC, ion mobility, and NMR spectroscopy require well-characterized glycan standards that must be pure enough, which is challenging, time-consuming, and costly to obtain. In particular, NMR data are difficult to interpret since glycans involve many carbons and protons with similar chemical environments. Nonetheless, the use of HPLC for glycan quantitation following glycan characterization via mass spectrometry is the most powerful and optimal combination. Electrospray ionization mass spectrometry is noted for multiple dissociation techniques, minimal sample consumption, short acquisition time, high sensitivity, high mass accuracy, and high resolution. However, the ionization efficiency and fragmentation of free glycans via mass spectrometry is relatively poor compared to tagged glycans. Therefore, to further aid in the analysis of glycans, many research labs have adopted the development and use of tagging reagents. Yet, these tagging reagents fail to offer simultaneous glycan quantitation and characterization capabilities and involve additional challenges, such as poor chemical stability and limited analysis towards a single category of glycans. Moreover, in order to compensate for the presence of proteinaceous amines, certain tagging reagents that recruit an NHS-carbamate group for the rapid tagging functionality require a large amount of reagent by as much as 133-fold of what is required for labeling glycans. Consequently, a novel tagging reagent for the improved analysis of glycans are desired.

### SUMMARY OF THE INVENTION

The present invention provides compounds useful for the quantitation and/or characterization of glycans and other similar molecules. Such compounds include compounds of formula (Ia) or (Ib)

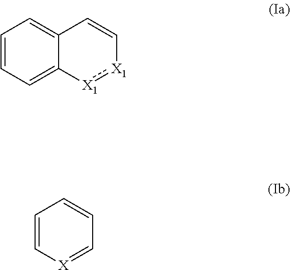

substituted with at least a TEMPO group or analog thereof and a coupling site wherein X¹, X² and X may be one of NH, N-alkyl, O, NO, C═O, CH, N or CR⁹.

Other such compounds include compounds having the following structures:

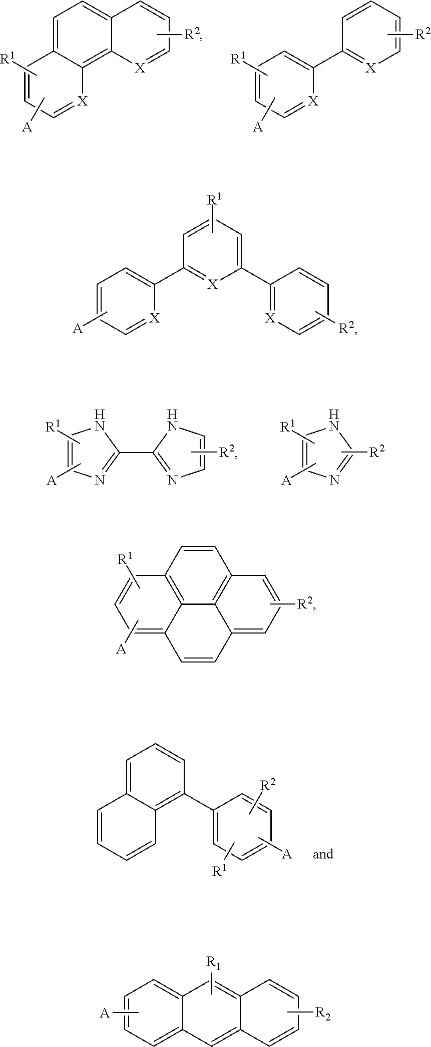

wherein R¹ is a glycan labeling site and R² and A may independently be hydrogen or a substituent.

The present invention is also directed to methods of quantitating and/or characterizing a glycan comprising obtaining a compound and contacting the compound with the glycan.

### BRIEF DESCRIPTION OF THE DRAWINGS

The drawings described below are for illustrative purposes only and are not intended to limit the scope of the invention.

FIG. 1 depicts an exemplary novel fluorescent tag (Glyc•RadiFluor) coupled with a free radical mechanistic approach towards glycan characterization and the other important sites for glycan analysis via HILIC-ESI⁺ MS.

FIG. 2 depicts the synthetic pathway for a novel fluorescent tag (Glyc•RadiFluor).

FIG. 3 depicts an ¹H NMR spectrum of 7-Amino- N-[2-(diethylamino)ethyl]-5-(TEMPO)methyl -2-quinolinecarboxamide.

FIG. 4 depicts a ¹³C NMR spectrum of 7-Amino- N-[2-(diethylamino)ethyl]-5-(TEMPO)methyl -2-quinolinecarboxamide.

FIG. 5 depicts a mass spectrum of 7-Amino- N-[2-(diethylamino)ethyl]-5-(TEMPO)methyl -2-quinolinecarboxamide in which this compound is detected as m/z 456.

FIG. 6 depicts a MS² spectrum of 7-Amino- N-[2-(diethylamino)ethyl]-5-(TEMPO)methyl -2-quinolinecarboxamide after being subjected to collisional-induced dissociation yields the loss of TEMPO and the remaining fragment of m/z 300.

FIG. 7 depicts a mass spectrum of the proton adduct of the conjugate which is detected as m/z 782.

FIG. 8 depicts a MS² spectrum of the conjugate after being subjected to collisional-induced dissociation yields the loss of TEMPO and the remaining fragment of m/z 626.

FIG. 9 depicts a MS³ spectrum of the conjugate after being subjected to collisional-induced dissociation yields fragmentations of the labeled lactose.

FIG. 10 depicts a calibration curve of 7-Amino- N-[2-(diethylamino)ethyl]-5-(TEMPO)methyl-2-quinolinecarboxamide in the nM range (NanoDrop™) fluorescence assay of the fluorescent tag from a range of 500 nM to 100 μM on the ThermoFisher Scientific™ NanoDrop™. The excitation wavelength is set to 365±10 nm and the emission wavelength is set to 520 nm.

FIG. 11 depicts calibration curve of 7-Amino- N-[2-(diethylamino)ethyl]-5-(TEMPO)methyl -2-quinolinecarboxamide in the nM range (Cary Eclipse®) including a fluoresence assay of the fluorescent tag between a concentration range of 1 nM to 1 μM on the Agilence Cary Eclipse®. The excitation wavelength is set to 390±5 nm and the emission wavelength is set to 520±5 nm.

FIG. 12 depicts an overlay of the fluorescence detection at 515 nm following excitation at 385 nm and reveals the separation via ultra-high performance liquid chromatography of a mixture of six reagent-labeled analytes on both columns: (in black) Glycan BEH Amide and (in red) Discovery C₁₈.

FIG. 13 depicts a spectrum of the total ion current caused by heated-electrospray ionization on the analytes separated by the Glycan BEH Amide column.

FIG. 14 depicts a mass spectrum at 8.53 min which relates to the results shown in FIG. 13 and reveals the expected m z peaks for the reagent-labeled maltose; [M+H]⁺=782.45 and [M+2H]²⁺=391.73.

FIG. 15 depicts a mass spectrum at 11.31 min which relates to the results shown in FIG. 13 and reveals the expected m z peaks for the reagent-labeled maltotriose; [M+H]⁺=944.51 and [M+2H]²⁺=472.76.

FIG. 16 depicts a mass spectrum at 14.31 min which relates to the results shown in FIG. 13 and reveals the expected m z peaks for the reagent-labeled maltotetraose; [M+H]⁺=1106.56 and [M+2H]²⁺=553.78.

FIG. 17 depicts a mass spectrum at 16.96 min which relates to the results shown in FIG. 13 and reveals the expected m z peaks for the reagent-labeled maltopentaose; [M+H]⁺=1268.61 and [M+2H]²⁺=634.81.

FIG. 18 depicts a mass spectrum at 19.15 min which relates to the results shown in FIG. 113 and reveals the expected m z peaks for the reagent-labeled maltohexaose; [M+H]⁺=1430.67 and [M+2H]²⁺=715.84.

FIG. 19 depicts a mass spectrum at 21.06 min which relates to the results shown in FIG. 13 and reveals the expected m z peaks for the reagent-labeled maltoheptaose; [M+H]⁺=1592.72 and [M+2H]²⁺=796.87.

FIG. 20 depicts a fluorescence chromatogram showing the separation for a ˜46 pmol injection of six GRF-derivatized maltosaccharides of variable lengths that were prepared in an equimolar mixture (˜7.7 pmol per analyte); the peaks are identified as follows: (1) maltose, (2) maltotriose, (3) maltotetraose, (4) maltopentaose, (5) maltohexaose, and (6) maltoheptaose. Based on the peak integrations (see Table 2), the average analyte quantity is 7.7±0.2 pmol.

FIG. 21 depicts the nomenclature for the glycan fragmentation ions.

FIG. 22 depicts a novel nomenclature for the reducing end open-chain isomer fragmentation ions.

FIGS. 23A and 23B depict: (A) the fragmentation patterns for the singly-protonated GRF-derivatized maltoheptaose, and (B) the HCD spectrum with NCE of 30 (arb) for the singly-protonated GRF-derivatized maltoheptaose; the fragmentations between ^{0,2}X and Z are associated with Z-nH₂O—ₓOH. (PIS and IS are abbreviations for the partial ionization site and ionization site, respectively in this and other figures.)

FIGS. 24A and 24B depict: (A) the fragmentation patterns for the singly-methylated GRF-derivatized maltoheptaose, and (B) the HCD spectrum with NCE of 30 (arb) for the singly-methylated GRF-derivatized maltoheptaose.

FIG. 25 depicts the HILIC separation and fluorescence detection of a sample containing unknown quantities of the branched isobaric glycans lacto-N-difucohexaose I (LNDFH I; peak 2) and lacto-N-difucohexaose II (LNDFH II; peak 1) after labeling with GRF.

FIGS. 26A, 26B, 26C and 26D depict: (A) the fragmentation patterns for the singly-protonated GRF-derivatized LNDFH II, (B) the HCD spectrum with NCE of 28 (arb) for the singly-protonated GRF-derivatized LNDFH II, (C) the fragmentation patterns for the singly-protonated GRF-derivatized LNDFH I, and (D) the HCD spectrum with NCE of 28 (arb) for the singly-protonated GRF-derivatized LNDFH I.

FIGS. 27A, 27B, 27C and 27D depict: (A) the fragmentation patterns for the singly-methylated GRF-derivatized LNDFH II, (B) the HCD spectrum with NCE of 32 (arb) for the singly-methylated GRF-derivatized LNDFH II, (C) the fragmentation patterns for the singly-methylated GRF-derivatized LNDFH I, and (D) the HCD spectrum with NCE of 32 (arb) for the singly-methylated GRF-derivatized LNDFH I.

FIG. 28 depicts a fluorescence chromatogram of the neutral GRF-derivatized dextran ladder separated via hydrophilic interaction liquid chromatography (HILIC).

FIG. 29 depicts an example logarithmic plot of the glucose unit value versus the retention time for the neutral GRF-derivatized dextran ladder that is specific for HILIC-FLD analysis.

FIG. 30 depicts a fluorescence chromatogram depicting the separation of GRF-derivatized RNase B high-mannose N-glycans; the peaks are identified as follows: (1) Man₅(GlcNAc)₂, (2) Man₆(GlcNAc)₂, (3) Man₇(GlcNAc)₂, (4) Man₉(GlcNAc)₂, (5) Man₉(GlcNAc)₂.

FIGS. 31A and 31B depict: (A) the HCD spectrum with NCE of 25 (arb) for the singly-protonated GRF-derivatized Man₅(GlcNAc)₂, and (B) the HCD spectrum with NCE of 28 (arb) for the singly-methylated GRF-derivatized Man₅(GlcNAc)₂.

FIGS. 32A and 32B depict: (A) the HCD spectrum with NCE of 25 (arb) for the singly-protonated GRF-derivatized Man₆(GlcNAc)₂, and (B) the HCD spectrum with NCE of 28 (arb) for the singly-methylated GRF-derivatized Man₆(GlcNAc)₂.

### DETAILED DESCRIPTION OF THE INVENTION

It is to be understood that the terminology employed herein is for the purpose of describing particular embodiments, and is not intended to be limiting. Further, although any methods, devices and materials similar or equivalent to those described herein can be used in the practice or testing of the invention, certain methods, devices and materials are now described.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The disclosure is further illustrated by the following descriptions, which are not to be construed as limiting this disclosure in scope or spirit to the specific descriptions herein described. It is to be understood that the descriptions are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims. For example, each embodiment disclosed herein is contemplated as being applicable to each of the other disclosed embodiments.

The present invention relates to methods and compositions for glycan analysis of complex solutions, including proteins and cells in a biological sample.

Provided is a compound of formula (Ia) or (Ib)

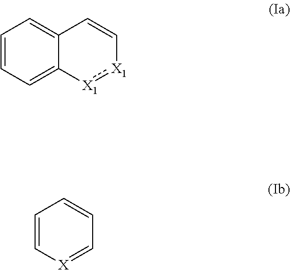

substituted with R¹ and A, and optionally substituted with R²,
wherein
[symbol] is a single or a double bond;
if [symbol] is a double bond, then:
X¹ is C and X² is C or N, or
X² is C and X¹ is N;
if [symbol] is a single bond, then:
X¹ is NH, N-alkyl, O, or NO and X² is C═O, or
X¹ is NH, N-alkyl, or NO and X² is O, or
X¹ is C═O and X² is NH, N-alkyl, O, or NO, or
X¹ is O and X² is NH, N-alkyl, or NO;
X is CH, N or CR⁹;
R¹ is selected from the group consisting of:

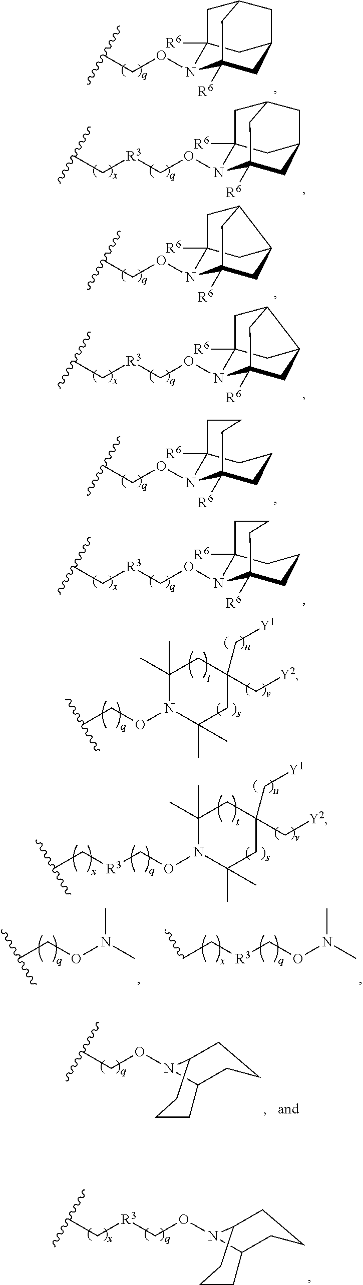

or optionally, a
derivative thereof;
Y¹ and Y² are each independently selected from the group consisting of:
H, O, OH, CH₃, N₃, CN, NH₂, R³—(CH₂)_{y}CO(CH₂)ₚCH₃, R³—(CH₂)_{y}COH, R³—(CH₂)_{y}—NH₂, N[(CH₂)_{y}(CH₃)]₂, R³—(CH₂)_{y}—N[(CH₂)ₚCH₃]₂, COOH, R³—(CH₂)_{y}—COOH, R³—(CH₂)_{y}—COO(CH₂)ₚCH₃, R³—(CH₂)_{y}—CN, R³—(CH₂)_{y}—N₃,

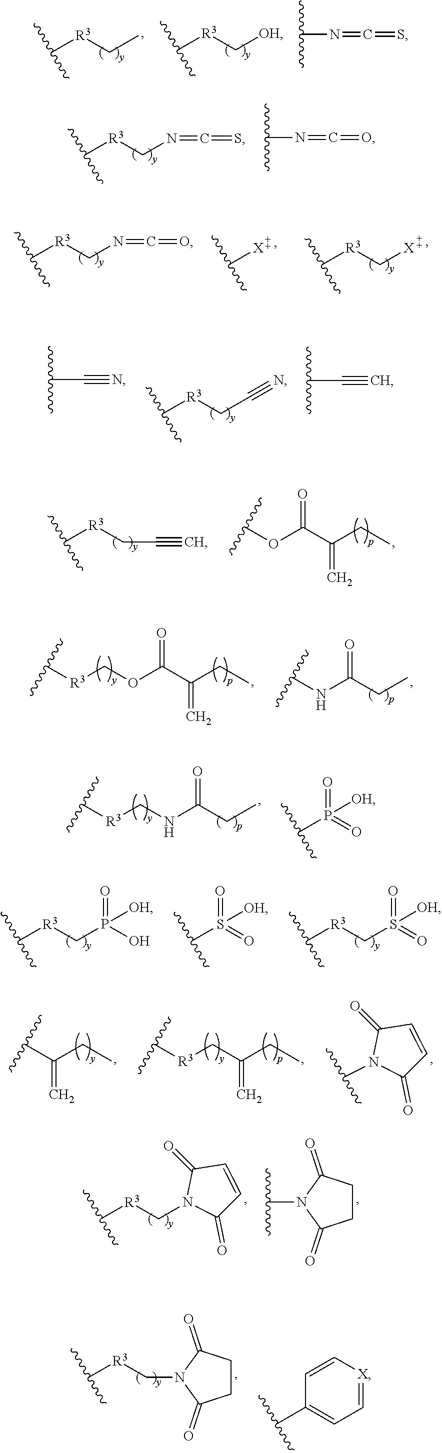

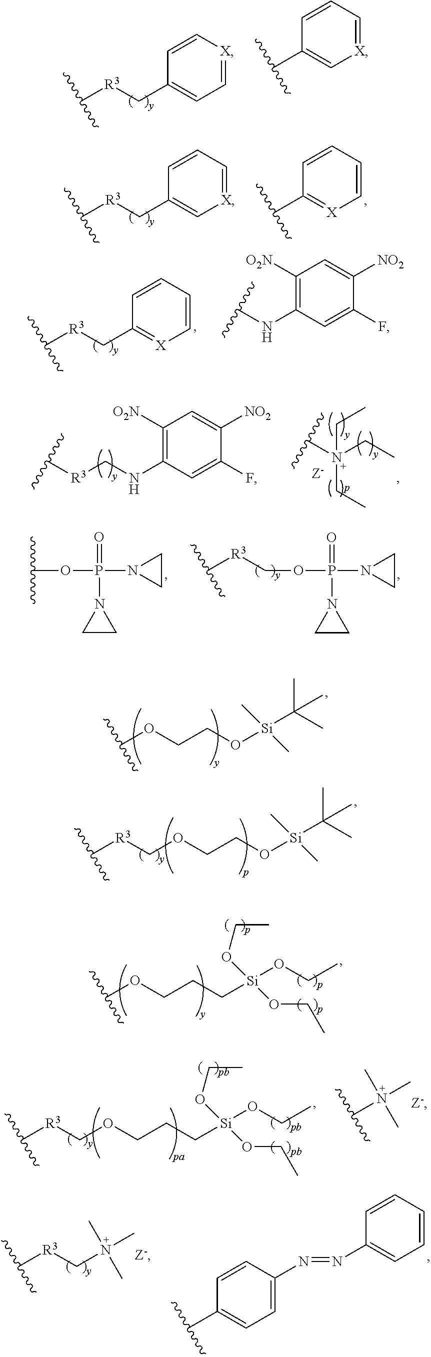

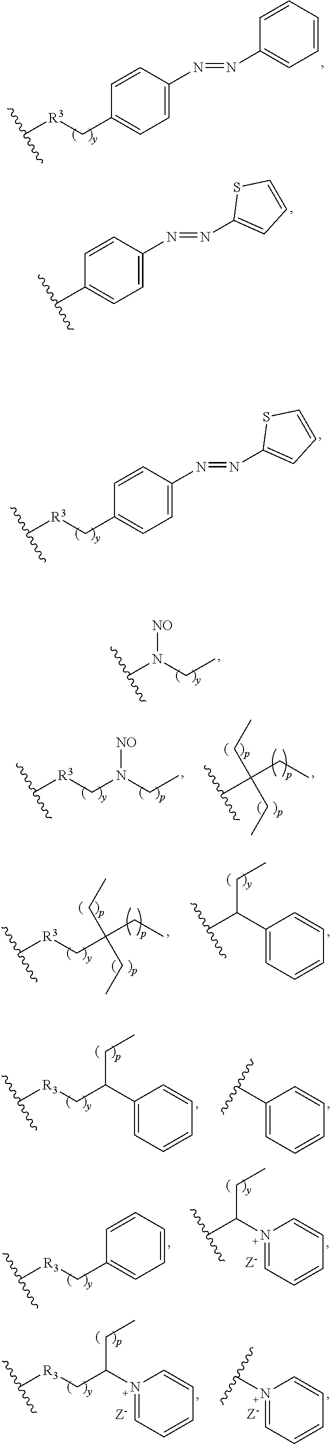

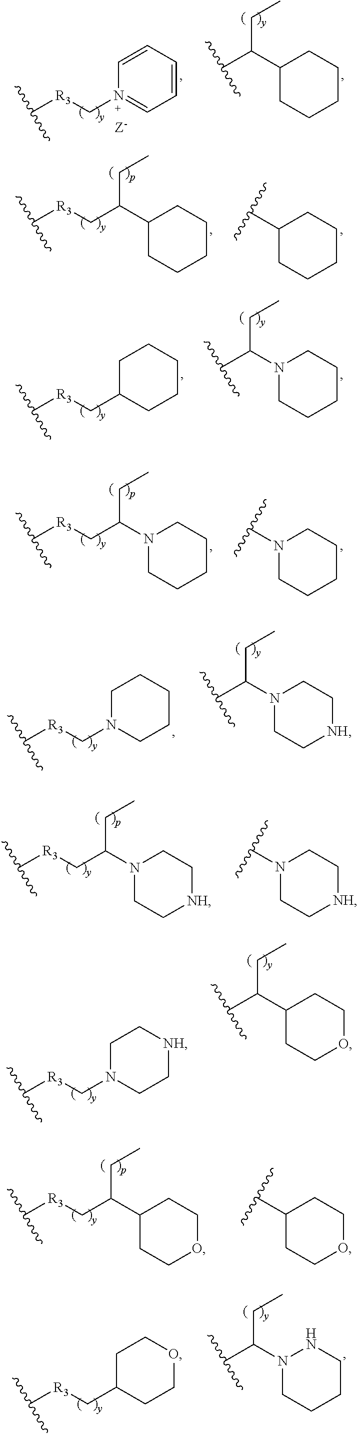

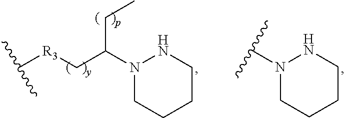

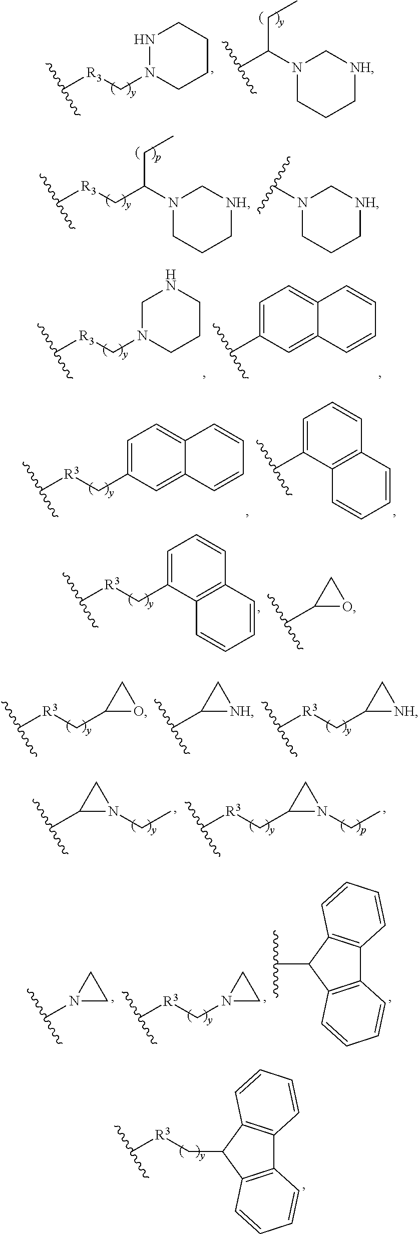

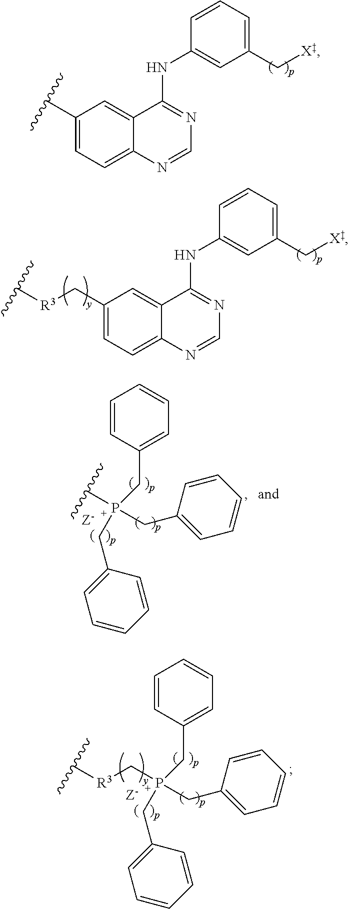

or, a and v are 0, and Y¹ and Y² form a structure together selected from the group consisting of

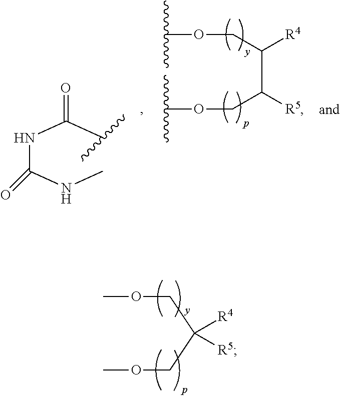

or, Y¹ and/or Y² function as a linker to a group independently selected from the groups defined in R¹ wherein the linker is selected from the group consisting of:

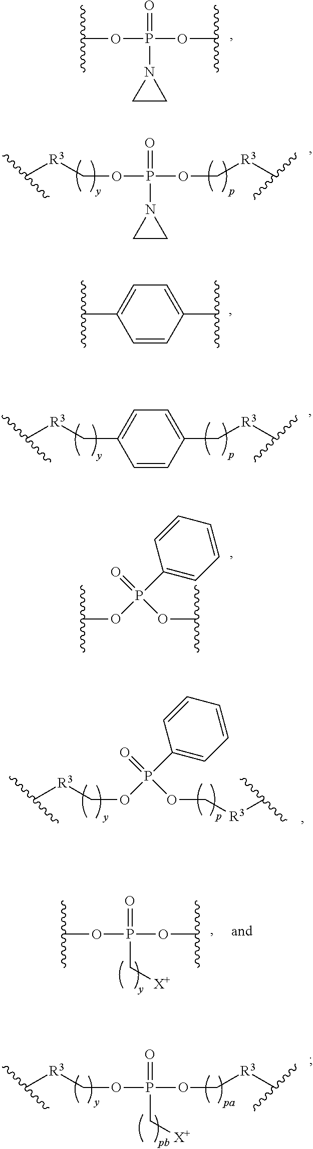

with the proviso that if Y¹ or Y² is a carbonyl, then the other is not a carbonyl,
X^{‡} is a halogen;
Z is a counter ion;
R² is selected from the group consisting of

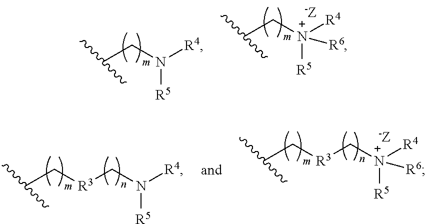

A is selected from the group consisting of

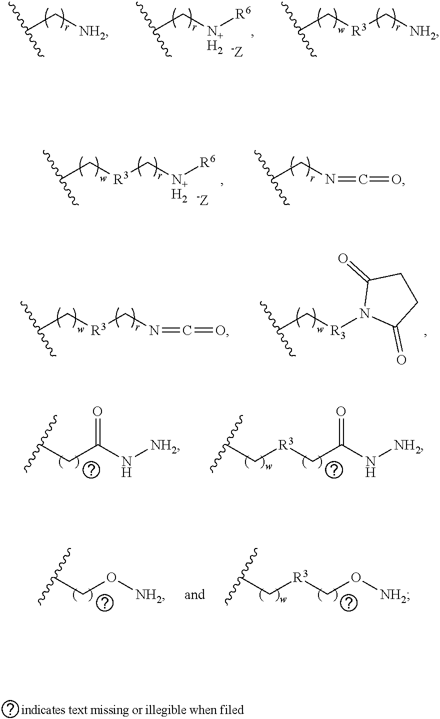

R³ are each independently selected from the group consisting of substituted nitrogen, oxygen, carbonyl, amide, ester, ether, urea, hydrazide, carbamate, carbonate, thiocarbonate, thiol, thiourea, sulfur, sulfoxide, and sulfone;
R⁴, R⁵, and R⁶ are each independently selected from the group consisting of H, OH, O, CN, N₃, COOH, alkyl, t-butyl, sec-butyl, isobutyl, isopropyl, tetrahydropyran, alkyl amino, alkylsulfonic acid, alkyl phosphonic acid, R³—(CH₂)_{y}CO(CH₂)ₚCH₃, R³—(CH₂)_{y}COH, NNH₂, R³—(CH₂)_{y}—NH₂, N[(CH₂)_{y}(CH₃)]₂, R³—(CH₂)_{y}—N[(CH₂)ₚCH₃]₂, R³—(CH₂)_{y}—COOH, R³—(CH₂)_{y}—COO(CH₂)ₚCH₃, R³—(CH₂)_{y}—CN, R³—(CH₂)_{y}—N₃, CH₃,

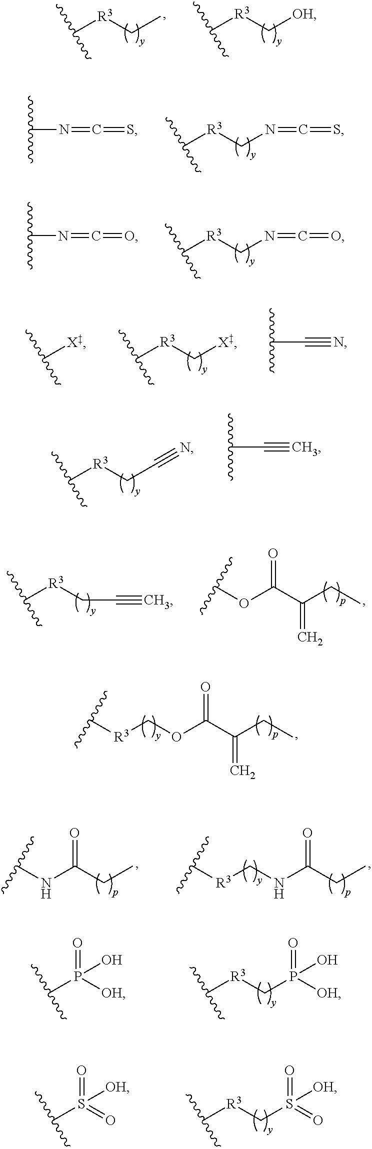

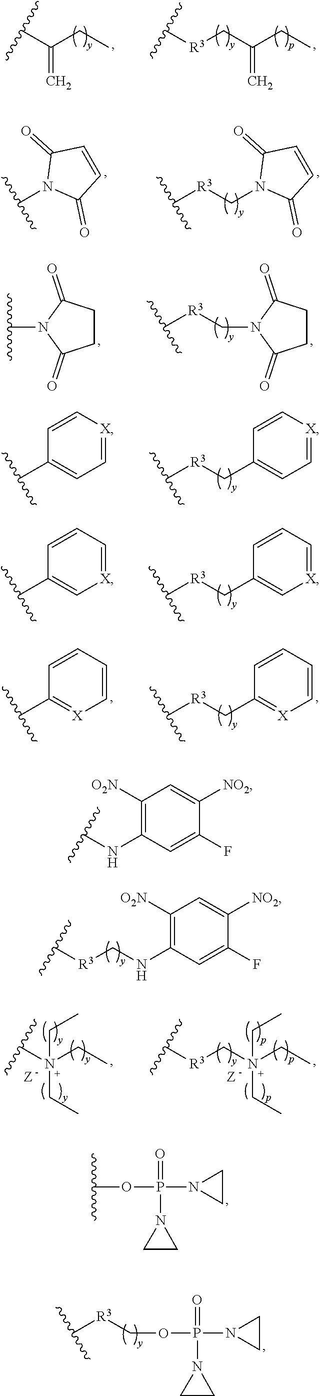

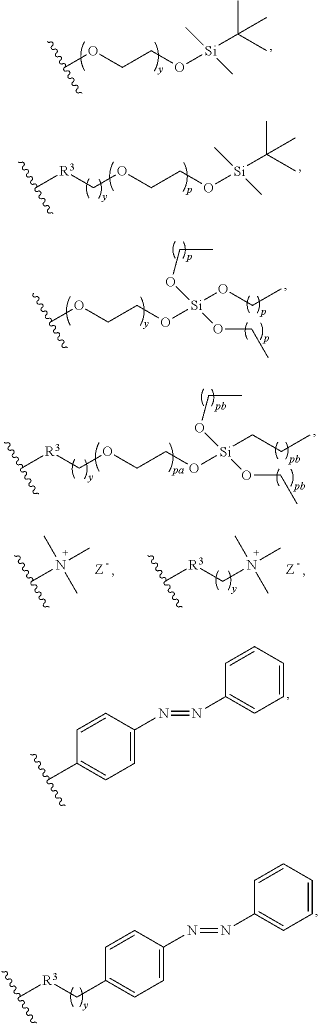

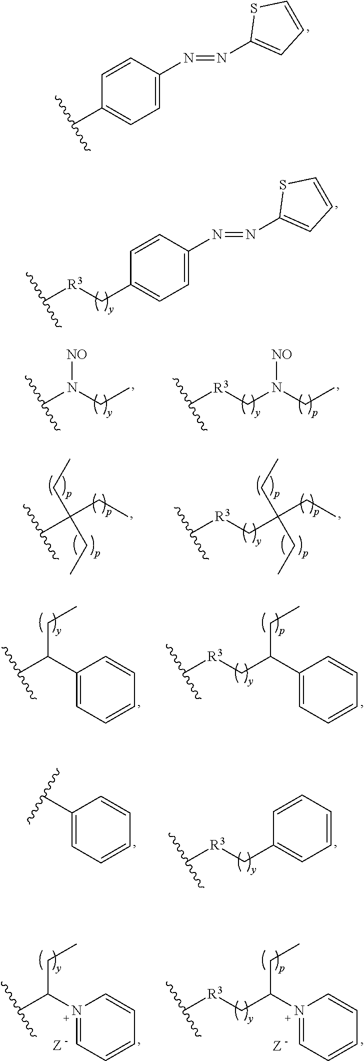

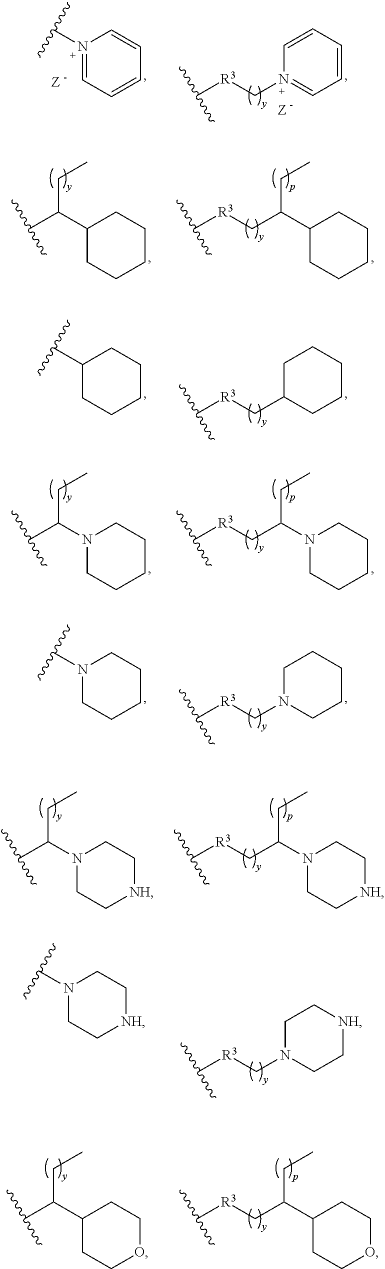

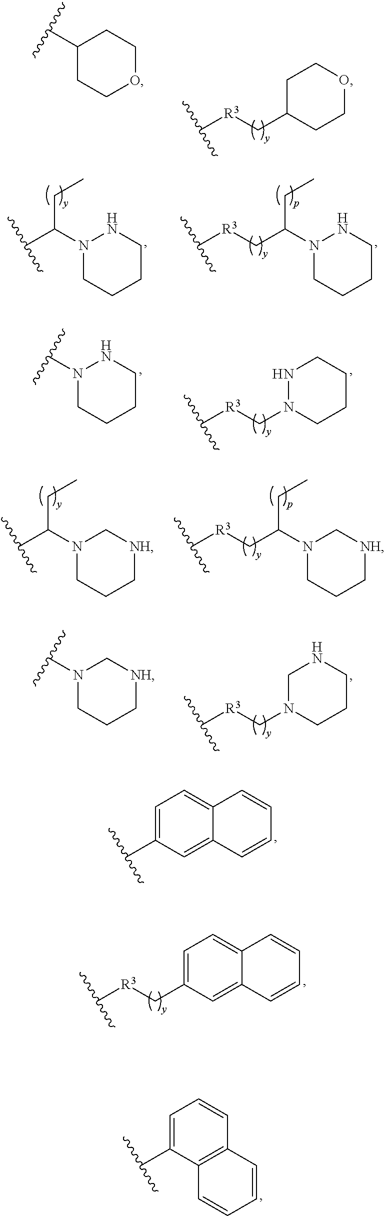

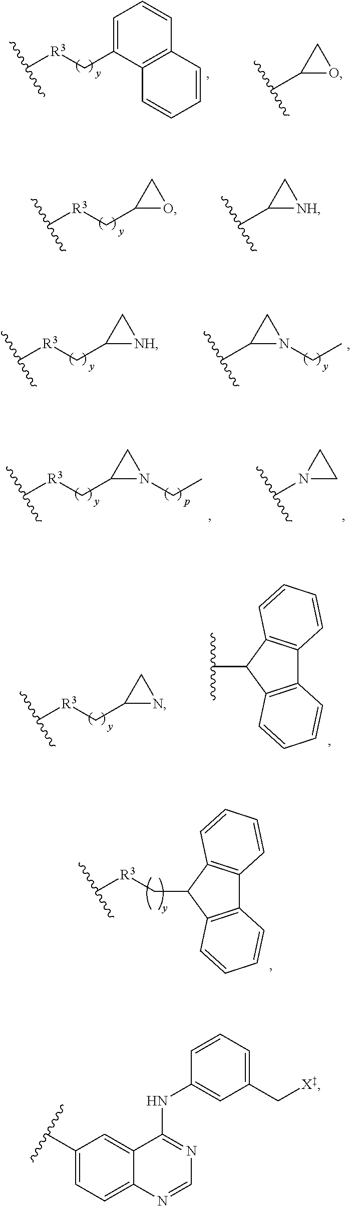

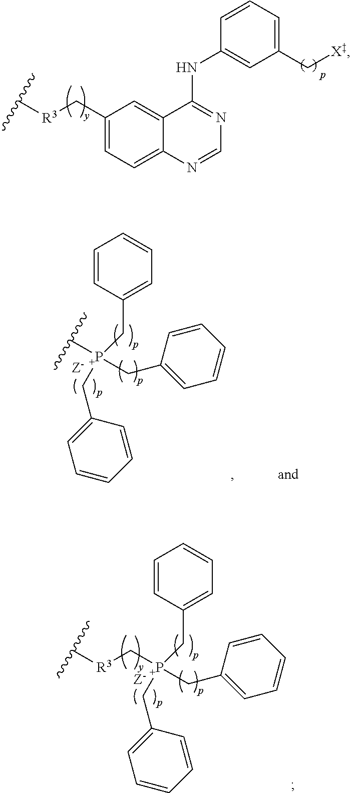

or R⁴ and R⁵ together with the nitrogen to which they are attached may form an optionally substituted 5- to 8-membered saturated or partially unsaturated ring;
or R⁴ and/or R⁵ function as a linker to a group independently selected from a group defined in R¹ wherein the linker is selected from the group consisting of:

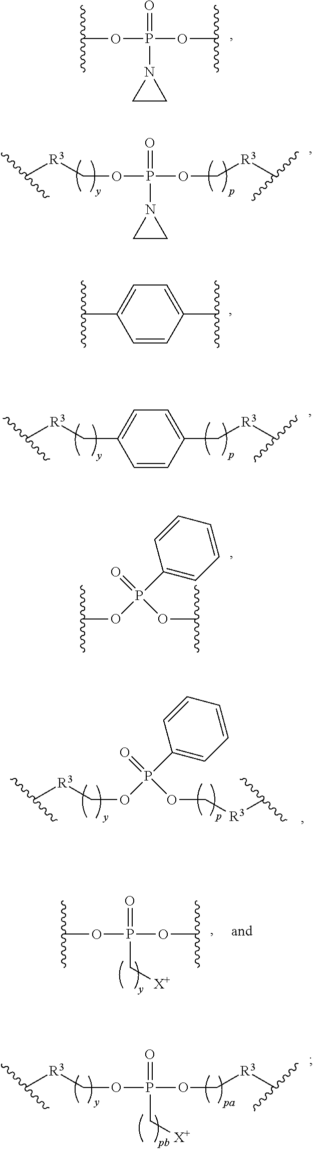

x, q, t, v, s, u, m, n, r, w, y, p, pₐ, and p_{b} are each independently an integer from 0 to 24;
R⁷ is a secondary, tertiary, or reduced amine, an ether, an alcohol;
R⁸ is an alkylated or hydrogenated imide, or an oxygen;
R⁹ is

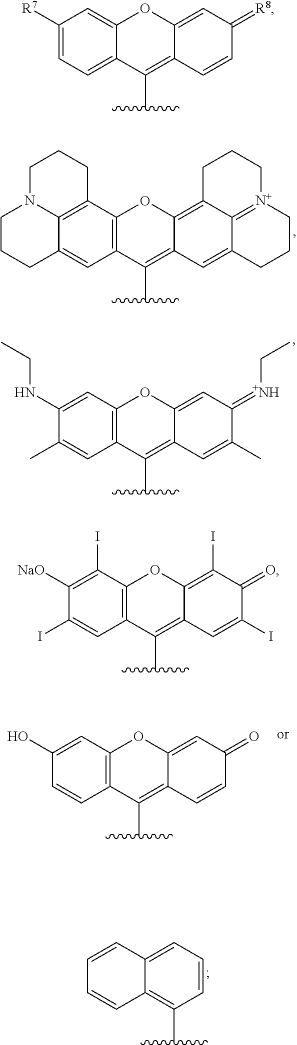

wherein any hydrogen atom may be substituted with a fluorine, deuterium, and tritium atom,
with the proviso that if the structure is a compound of formula (Ib), then formula (Ib) is substituted with R¹, R² and A;
with the proviso that if X₁ is N and X₂ is C, and R₁ is

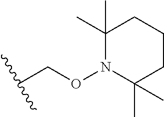 [SMILES: *CON1C(C)(C)CCCC1(C)C]

and one of R² or A is H, then each of R² or A is a substituent other than

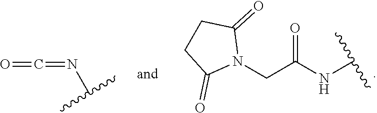

In embodiments, R² and A are substituents other than

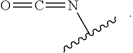 [SMILES: *N=C=O]

In embodiments, R² and A are substituents other than

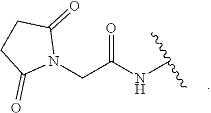 [SMILES: *NC(=O)CN1C(=O)CCC1=O]

In embodiments, R² and A are substituents other than

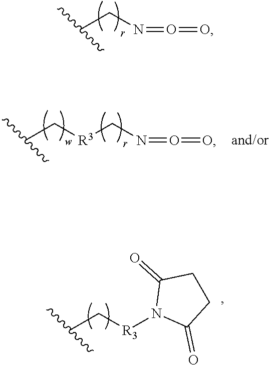

optionally only when one of R² or A is hydrogen.

In some embodiments, the compound is substituted with R². In some embodiments, the compound is substituted with A. In some embodiment, the compound is substituted with R² or A. In other embodiment, the compound is substituted with R² and A.

In one embodiment, R¹, R² and A are not an isocyanate or a succidimidyl carbamate. In another embodiment, R¹, R² and A are not an isocyanate or a derivate thereof. In another embodiment, R¹, R² and A are not a succidimidyl carbamate or a derivate thereof.

In some embodiments, the compound has formula (Ic)

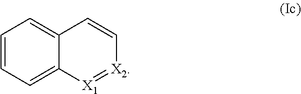

In embodiments, the compound has formula (Id), formula (Ie), or formula (If)

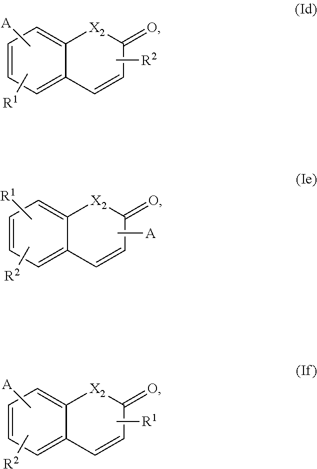

wherein X² is NH, N-alkyl, or NO.

In some embodiments, the compound is

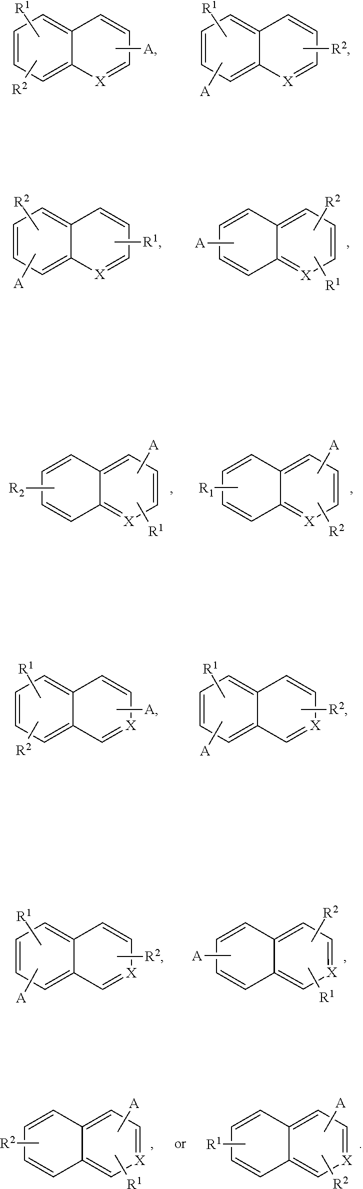

In some embodiments, the compound is

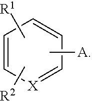

In embodiments, the compound is

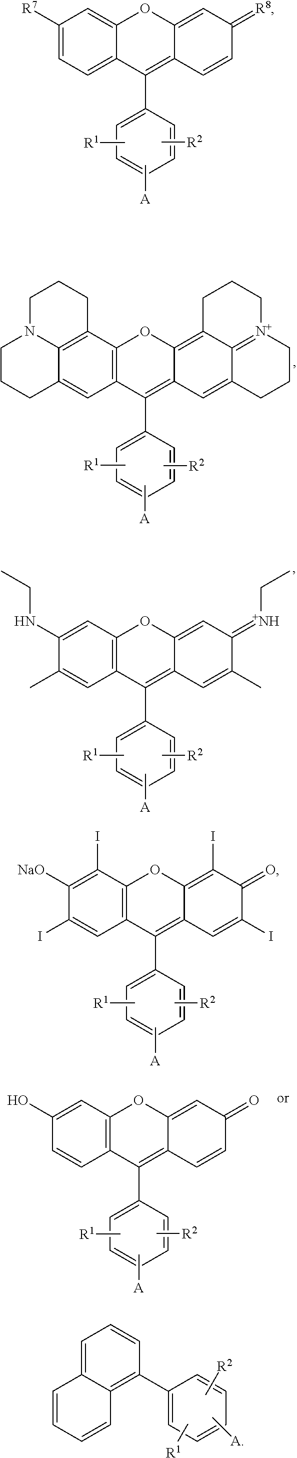

Provided is a compound which is

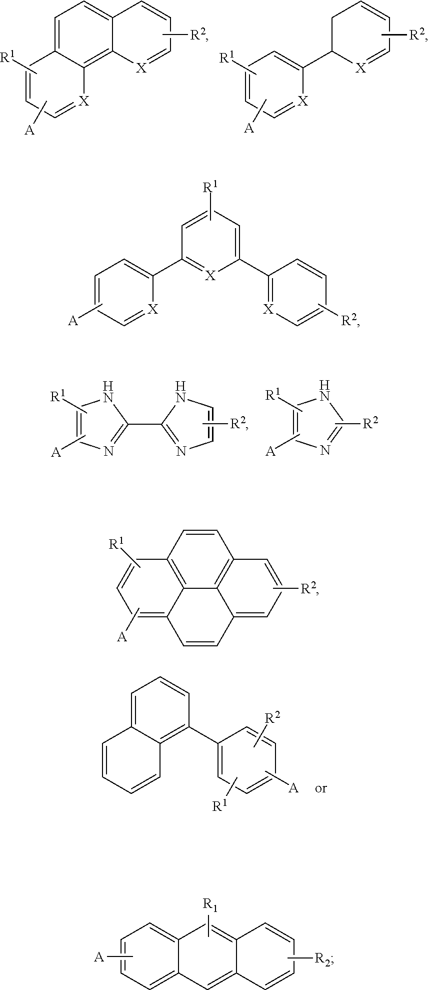

and X, A, R¹ and R² are defined as recited previously.

In embodiments, R₁ is

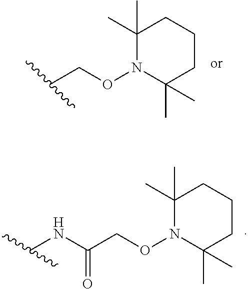 [SMILES: *CON1C(C)(C)CCCC1(C)C ; *NC(=O)CON1C(C)(C)CCCC1(C)C]

In embodiments R₂ is

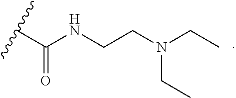 [SMILES: *C(=O)NCCN(CC)CC]

In embodiments A is

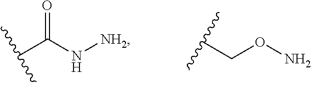

### or NH₂.

In embodiments X is CH, or N.

In one embodiment, R⁶ are each independently selected from the group consisting of: H, alkyl, t-butyl, sec-butyl, isobutyl, isopropyl, alkyl amino, alkylsulfonic acid, or alkyl phosphonic acid.

In embodiments, the compound is a fluorescence tag.

In embodiments, the compound is:

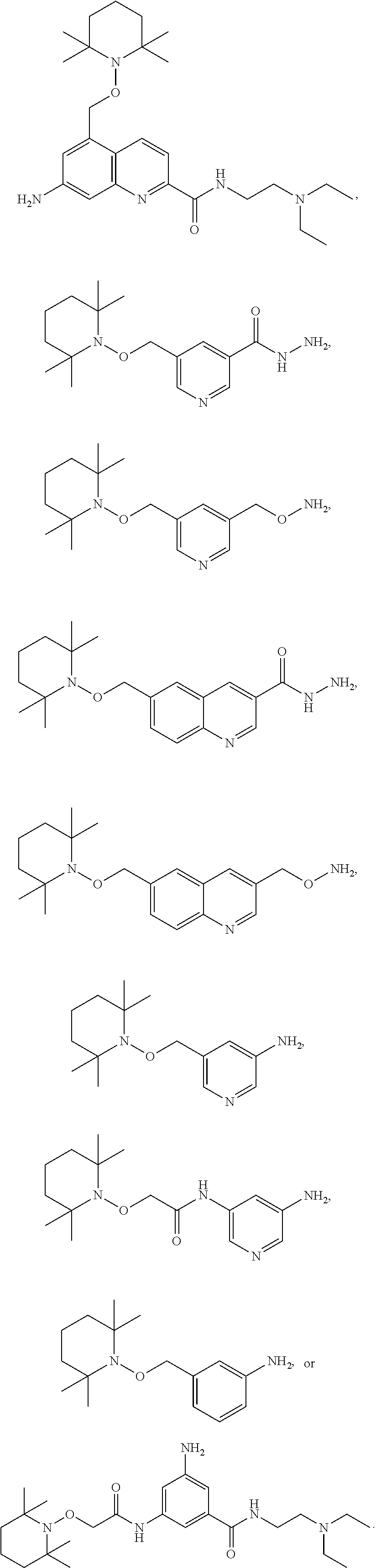

In embodiments, x, q, t, v, s, u, m, n, r, w, y, p, pₐ, and p_{b} are each independently an integer from 0 to 18, 0 to 12, 0 to 6, 0 to 4, 0 to 2, or 1 to 2.

In embodiments, A is a substituent other than

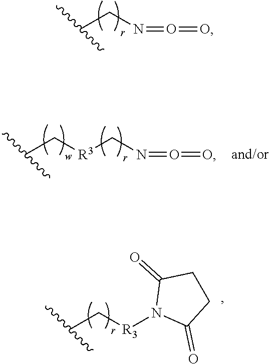

optionally when R² is a hydrogen.

Counter ions include the alkali metals lithium (Li⁺), sodium (Na⁺), potassium (K⁺), rubidium (R^{b+}), cesium (Cs⁺), and francium (Fr⁺). Additionally, counter ions may also include substituted or unsubstituted ammonium (NH₄⁺ or RNH₃⁺ or R₂NH₂⁺ or R₃NH⁺) and proton (H⁺). Counter ions further include the alkaline earth metals beryllium (Be²⁺), magnesium (Mg²⁺), calcium (Ca²⁺), strontium (Sr²⁺), barium (Ba²⁺), and radium (Ra²⁺) or any of the transition metals in any of their various oxidation states (for example, but not limited to, Cu⁺ and Cu²⁺) and any complex ions that they form. Counter ions may further include the halides fluoride (F⁻), chloride (Cl⁻), bromide (Br⁻), iodide (I⁻), and astatide (At⁻) or any phosphate (H₂PO₄⁻ or Li₂PO₄⁻ or Na₂PO₄⁻ or K₂PO₄⁻ or Rb₂PO₄⁻ or Cs₂PO₄⁻ or Fr₂PO₄⁻ or BePO₄⁻ or MgPO₄⁻ or CaPO₄⁻ or SrPO₄⁻ or BaPO₄⁻ or RaPO₄⁻ or HPO₄²⁻ or LiPO₄²⁻ or NaPO₄⁻ or KPO₄²⁻ or RbPO₄²⁻ or CsPO₄²⁻ or FrPO₄²⁻ or PO₄³⁻), any borate (H₂BO₃⁻ or Li₂BO₃⁻ or Na₂BO₃⁻ or K₂BO₃⁻ or Rb₂BO₃⁻ or Cs₂BO₃⁻ or Fr₂BO₃⁻ or BeBO₃⁻ or MgBO₃⁻ or CaBO₃⁻ or SrBO₃⁻ or BaBO₃⁻ or RaBO₃⁻ or HBO₃²⁻ or LiBO₃²⁻ or NaBO₃²⁻ or KBO₃²⁻ or RbBO₃²⁻ or CsBO₃²⁻ or FrBO₃²⁻ or BO₃³⁻), or any sulfate (HSO₄⁻ or LiSO₄⁻ or NaSO₄⁻ or KSO₄⁻ or RbSO₄⁻ or CsSO₄⁻ or FrSO₄⁻ or SO₄²⁻), or any sulfite (HSO₃⁻ or LiSO₃⁻ or NaSO₃⁻ or KSO₃⁻ or RbSO₃⁻ or CsSO₃⁻ or FrSO₃⁻ or SO₃²⁻), or any arsenate (H₂AsO₄⁻ or Li₂AsO₄⁻ or Na₂AsO₄⁻ or K₂AsO₄⁻ or Rb₂AsO₄⁻ or Cs₂AsO₄⁻ or Fr₂AsO₄⁻ or BeAsO₄⁻ or MgAsO₄⁻ or CaAsO₄⁻ or SrAsO₄⁻ or BaAsO₄⁻ or RaAsO₄⁻ or HAsO₄²⁻ or LiAsO₄²⁻ or NaAsO₄²⁻ or KAsO₄²⁻ or RbAsO₄²⁻ or CsAsO₄²⁻ or FrAsO₄²⁻ or AsO₄³⁻), or any carbonate (HCO₃⁻ or LiCO₃⁻ or NaCO₃⁻ or KCO₃⁻ or RbCO₃⁻ or CsCO₃⁻ or FrCO₃⁻ or CO₃²⁻), or any silicate (HSiO₃⁻ or LiSiO₃⁻ or NaSiO₃⁻ or KSiO₃⁻ or RbSiO₃⁻ or CsSiO₃⁻ or FrSiO₃⁻ or SiO₃²⁻), or any selenate (HSeO₄⁻ or LiSeO₄⁻ or NaSeO₄⁻ or KSeO₄⁻ or RbSeO₄⁻ or CsSeO₄⁻ or FrSeO₄⁻ or SeO₄²⁻), or any oxide (HPO₂⁻ or LiO₂⁻ or NaO₂⁻ or KO₂⁻ or RbO₂⁻ or CsO₂⁻ or FrO₂⁻ or O₂²⁻ or HO⁻ or LiO⁻ or NaO⁻ or KO⁻ or RbO⁻ or CsO⁻ or FrO⁻ or O²⁻), or any hypohalite (FO⁻ or ClO⁻ or BrO⁻ or IO⁻ or AtO⁻), any halite (FO₂⁻ or ClO₂⁻ or BrO₂⁻ or IO₂⁻ or AtO₂⁻), or any halate (FO₃⁻ or ClO₃⁻ or BrO₃⁻ or IO₃⁻ or AtO₃⁻), any perhalate (FO₄⁻ or ClO₄⁻ or BrO₄⁻ or IO₄⁻ or AtO₄⁻), or any acetate (CF₃COO⁻ or CCl₃COO⁻ or CBr₃COO⁻ or CI₃COO⁻ or CH₃COO), or any sulfide (HS⁻ or LiS⁻ or NaS⁻ or KS⁻ or RbS⁻ or CsS⁻ or FrS⁻ or S²⁻), formate (CHOO⁻), cyanate (CN⁻), nitrate (NO₃⁻), nitrite (NO₂⁻). In embodiments, the counter ion may be any ion that is of opposite charge of the species presented. For example, if the species has a 2+ charge, the counter ion Z can be any ion from any group that has an opposite charge in the form of 2−.

Provided is a method of quantitating and/or characterizing a glycan comprising:
a) obtaining the compound of recited above;
b) contacting this compound with the glycan, thereby forming a labeled glycan.

In embodiments, the method further comprises dissociation of the labeled glycan to form glycan fragmentations.

In some embodiments the dissociation is collisional-induced dissociation (CID) or higher-energy collision dissociation (HCD).

In embodiments, the method further comprises thereby quantitating and/or characterizing a glycan.

In some embodiments, the glycan comprises a reducing terminus.

In some embodiments, the glycan is other than an N-glycan.

In embodiments, the method further comprises analyzing the labeled glycan or glycan fragmentations with an instrument which is a fluorimeter, mass spectrometer or liquid chromatography instrument, optionally with an instrument capable of detecting fluorescence and/or absorbance.

The instrument may be an Ultra-Performance Liquid Chromatography (UPLC), a linear quadrupole ion trap (LTQ-XL) mass spectrometer, a Q Exactive Orbitrap mass spectrometer, or a liquid chromatography-mass spectrometry (LC-MS).

In some embodiments, the instrument is equipped with an electrospray ionization (ESI) or heated-electrospray ionization (HESI) source and/or with a fluorescence detector.

Provided is a compound for use in glycan detection or glycan quantitation or for use in the preparation of a labeled glycan or glycan fragmentations.

The present invention, in one aspect, provides a method for glycan analysis of a sample, the method comprising the steps of contacting the glycan with a compound of the invention and scanning the analyte by mass spectrometry to detect and identify the presence of glycans.

The present invention also provides for quantitating and/or characterizing of a sample comprising compounds such as biomolecules, non-biomolecules, proteins, peptides, or amino acids in the same manner as glycans. In some embodiments, these compounds have a similar functional group as the reducing termini of glycans.

In one embodiment, the sample comprises at least one protein solution. In one embodiment, the sample comprises at least one population of cells.

In one exemplary method of the invention, a herein disclosed compound is contacted with a glycan of interest. In this step, the chemical reaction may form a covalent bond between the glycan of interest and the compound, thereby labeling the glycan. In another exemplary step of the invention, a liquid chromatography instrument equipped with a fluorescence detector is used to quantitate the amount of glycan in a given sample via the fluorescence caused by the labeled glycan. Next, for example, a mass spectrometer is used to induce fragmentations on the glycan, which then provides data which can be used to characterize the glycan of interest.

### Definitions

“Glycans” are sugars, for example oligosaccharides and polysaccharides, which can be monomers or polymers of sugar residues often joined by glycosidic bonds. In some embodiments, the terms “glycan”, “oligosaccharide” and “polysaccharide” may refer to the carbohydrate portion of a glycoconjugate (e.g., glycolipid, glycoprotein, or proteoglycan). A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′-sulfo N-acetylglucosamine, etc.). The term “glycan” includes homo and heteropolymers of sugar residues. The term “glycan” also encompasses a glycan component of a glycoconjugate (e.g., of a glycoprotein, glycolipid, proteoglycan, etc.). The term also encompasses free glycans, including glycans that have been cleaved or otherwise released from a glycoconjugate. Glycan may include an O-linked glycan or an N-linked glycan. The O-linked glycan has a structure in which a glycan is linked to a side chain of an amino acid residue serine (Ser) or threonine (Thr) in a protein via a —OH group included in the amino acid side chain. The O-linked glycans are classified into one to eight types according to the core structure. The N-linked glycan refers to a glycan that is linked to a nitrogen atom of an amide group in a side chain of an asparagine residue (Asn) of a protein. The N-linked glycans include glycans that form branches with mannose used as a base point, and examples thereof include two branched, three branched, and four branched glycans, and the like. Also, the N-linked glycans can be classified into basic, high mannose, hybrid, and complex types, and the like according to the structures thereof.

As used herein, the term “alkyl”, alone or in combination with other groups, refers to a branched or straight-chain monovalent saturated aliphatic hydrocarbon radical of one to twenty carbon atoms, in one embodiment one to sixteen carbon atoms, in another embodiment one to ten carbon atoms. In some embodiments, the alkyl is a lower alkyl.

The term “lower alkyl”, alone or in combination with other groups, refers to a branched or straight-chain alkyl radical of one to nine carbon atoms, in one embodiment one to six carbon atoms, in another embodiment one to four carbon atoms, in a further embodiment four to six carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

As used herein, the term “alkoxy” means alkyl-O—; and “alkoyl” means alkyl-CO—. Alkoxy substituent groups or alkoxy-containing substituent groups may be substituted by, for example, one or more alkyl or halo groups.

As used herein, the term “halogen” means a fluorine, chlorine, bromine or iodine radical, or in some embodiments a fluorine, chlorine or bromine radical.

As used herein, a “ara-alkyl” refers to an alkyl group substituted with at least one aryl group. Similarly, as used herein, “heteroara-alkyl” group refers to an alkyl group substituted with at least one heteroaryl group.

The term “cycloalkyl” refers to a monovalent mono- or polycarbocyclic radical of three to ten, in one embodiment three to six carbon atoms. This term is further exemplified by radicals such as cyclopropyl, cyclobutyl, cyclopentyl, cyclohexyl, cycloheptyl, norbornyl, adamantyl, indanyl and the like. In one embodiment, the “cycloalkyl” moieties can optionally be substituted with one, two, three or four substituents. Each substituent can independently be alkyl, alkoxy, halogen, amino, hydroxyl, aryl, heteroaryl or oxygen unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, 1H-indenyl and the like.

The alkyl, lower alkyl, aryl, and spirocycloalkyl groups, may be substituted or unsubstituted. Additionally, ara-alkyl and heteroara-alkyl groups may be substituted with substituents in addition to aryl or heteroaryl groups. When substituted, there will generally be, for example, 1 to 4 substituents present. These substituents may optionally form a ring with the alkyl, lower alkyl or aryl group with which they are connected. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl, in other embodiments, for example, methoxy and ethoxy), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. Examples of such groups include, but not limited to, pyridinyl, pyrazinyl, pyridazinyl, 1,2,3-triazinyl, 1,2,4-triazinyl, oxazolyl, thiazolyl, and the like.

The heteroaryl group described above may be substituted independently with one, two, or three substituents. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di- alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl, benzothiazoyl and carbolinyl).

Where any group has been referred to as optionally substituted, this group may be substituted or unsubstituted. Substitution may be by one or more of the specified substituents which may be the same or different. It will be appreciated that the number and nature of substituents will be selected to avoid any sterically undesirable combinations.

“Optionally substituted” as applied to any group means that the said group may if desired be substituted with one or more substituents, which may be the same or different. Examples of suitable substituents for “substituted” and “optionally substituted” moieties, include halo, deutero, C₁₋₆ alkyl or C₁₋₃ alkyl, hydroxy, C₁₋₆ alkoxy or C₁₋₃ alkoxy, cyano, amino, nitro or SF₅ (a known mimetic of NO₂), aryl, heteroaryl, heterocyclyl, C₃-C₆ cycloalkyl, C₁₋₃ alkylamino, C₂₋₆ alkenylamino, di-C₁₋₃ alkylamino, C₁₋₃ acylamino, di-C₁₋₃ acylamino, carboxy, C₁₋₃ alkoxycarbonyl, carbamoyl, mono-C₁₋₃ carbamoyl, di-C₁₋₃ carbamoyl or any of the above in which a hydrocarbyl moiety is itself substituted by halo. In groups containing an oxygen atom such as hydroxy and alkoxy, the oxygen atom can be replaced with sulphur to make groups such as thio (SH) and thio-alkyl (S-alkyl). Optional substituents therefore include groups such as S-methyl. In thio-alkyl groups, the sulphur atom may be further oxidised to make a sulfoxide or sulfone, and thus optional substituents therefore includes groups such as S(O)-alkyl and S(O)₂-alkyl.

Substituted groups thus include for example Cl, F, OMe, Me, COCH₃, CONH₂, NHC(O)CH(CH₃)₂, CO₂CH₂CH₃ etc. In the case of aryl groups, the substitutions may be in the form of rings from adjacent carbon atoms in the aryl ring, for example cyclic acetals such as O—CH₂—O.

The optional substituents for any alkyl, alkenyl, alkynyl, alkoxy, alkylene or alkenylene groups described herein may be selected from C₁-C₃ alkoxy, halogen, hydroxyl, thiol, cyano, amino, amido, nitro and SF₅, wherein the alkoxy may be optionally substituted with halogen. In particular, the optional substituents may be selected from halogen, hydroxyl, thiol, cyano, amino, amido, nitro and SF₅, more particularly fluorine or hydroxyl.

Generally, hydrogen is not considered a substituent. However, in select embodiments, A and/or R² is a hydrogen.

By any range disclosed herein, it is meant that all hundredth, tenth and integer unit amounts within the range are specifically disclosed as part of the invention. Thus, for example, 0.01 to 50 means that 0.02, 0.03 . . . 0.09; 0.1; 0.2 . . . 0.9; and 1, 2 . . . 49 unit amounts are included as embodiments of this invention.

In any of the disclosures herein, if a particular R group may be more than one functional group and the compound's structure has more than one of that particular R group, the R groups may each be a different functional group. For example, if a structure has two R⁴ groups and one is a hydrogen, the other may be an alkyl group. In a similar manner, each letter representing a number in the structures, if present multiple times in a single structure, may be different in each instance.

Compounds of the present invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms.

Compounds of the present invention can be prepared beginning with commercially available starting materials and utilizing general synthetic techniques and procedures known to those skilled in the art.

There is an extensive number of dissociation techniques via MS that have been noted, and yet, recent techniques are continuously being developed. For instance, collision-induced dissociation (CID) and infrared multiphoton dissociation (IRMPD) are commonly known to generate fragmentations via glycosidic bond cleavages. In comparison, ultraviolet photodissociation (UVPD) and higher-collision dissociation (HCD) have previously been shown to generate analyte fragmentations that are more information-rich. Radical-driven dissociation (RDD), electron-capture dissociation (ECD), electronic excitation dissociation (EED), electron transfer dissociation (ETD), and electron detachment dissociation (EDD), often grouped together as free radical-driven dissociation techniques, have similarly demonstrated great potential for glycan structural analysis. Nonetheless, UVPD, MSⁿ, ion-mobility mass spectrometry, and EDD remain the most powerful in discerning the mass spectra belonging to glycan isomers, especially stereoisomers such as anomers and epimers. As shown in the Examples to follow, by combining MSⁿ and CID with a reagent that includes a free radical precursor, glycan structural analysis can be feasibly, accurately, and rapidly performed. Gas-phase ion/ion reactions that internally take place within modified mass spectrometers that are capable of charge-inverting anionic analytes have been additionally demonstrated, showing the potential for future applications onto glycan structural analysis. Experiments in both the gas-phase and condensed-phase are being performed where a reagent, which employs a free radical precursor, forms a complex with the analyte of choice, allowing for free radical-directed fragmentations to subsequently take place. However, this approach does not currently allow for the sensitive detection and quantitation of the glycans to take place by fluorescence.

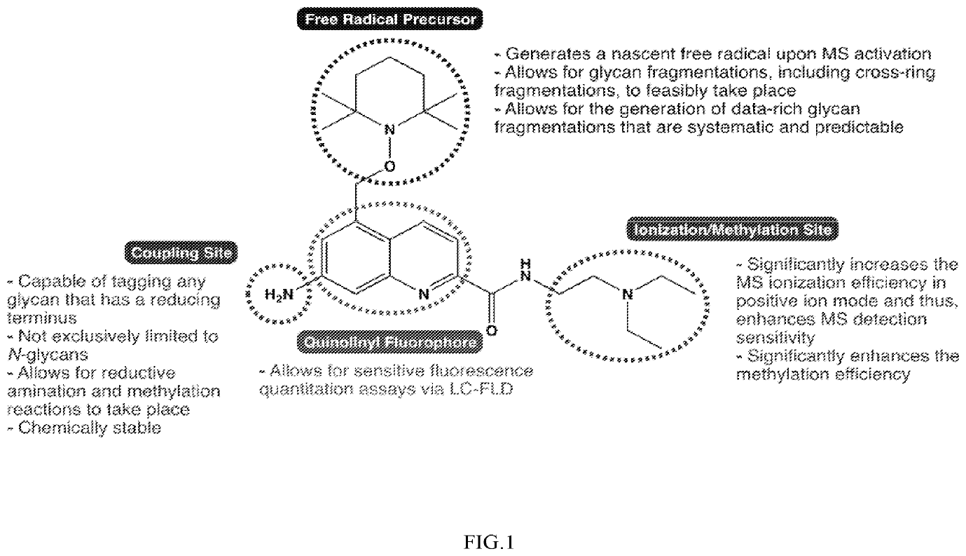

Recently, free radical-activated glycan sequencing reagents (FRAGS) were developed to react and covalently attach to glycans at their respective reducing termini. The FRAGS reagents enlist a free radical precursor (e.g., 2,2,6,6-tetramethyl-1-piperidinyloxy, or TEMPO, among other nitroxyl free radical precursors), which upon collisional activation of the tagged glycan, generates a localized nascent free radical that simultaneously induces predictable, diagnostic, and systematic glycan fragmentations. Furthermore, a basic pyridyl site for charge induction is recruited in the FRAGS reagents, in addition to the hydrazide or aminooxy labeling sites which react with the glycan reducing terminus. Previously, the first-generation FRAGS was methylated at the pyridine moiety (Me-FRAGS I) and was covalently attached to isomeric oligosaccharides with subtle differences. The methylation elicits a localized charge, thus preventing gas-phase glycan rearrangements that are often observed in the protonated species which involve mobile charges. Subsequently, MS³ collisional-induced dissociation (CID) spectra involving Me-FRAGS I had shown its ability to accurately and rapidly distinguish between nine isomeric disaccharides and two isomeric tetrasaccharides. Although the FRAGS-labeled glycans can be detected via liquid chromatography, they lack a fluorescently-active moiety which would substantially increase the sensitivity of liquid chromatography detection and quantitation via fluorescence. Moreover, the hydrazide and aminooxy labeling sites have a reduced coupling efficiency and stability relative to developing a tag with a free amino group as the coupling site. In particular, the hydrazide group has strong interactions with sodium ions following reductive amination, thus impeding the rate of methylation for fixed charge development. Additionally, an in-depth review of the tags currently on the market fails to offer simultaneous quantitation and characterization of glycans. Moreover, certain tags that enlist the NHS-carbamate functional group have poor stability upon the presence of atmospheric moisture. Thus, we have developed a tag which includes the following sites: (1) a free radical precursor that yields a nascent free radical that is capable of inducing systematic and predictable glycan fragmentations; (2) an ionization site that allows for a fixed charge to take place which results in enhanced ionization efficiency and methylation efficiency; (3) a fluorophore which allows for sensitive quantitation of tagged glycans; and (4) an amino coupling site which dramatically enhances the coupling efficiency between glycans (See FIG. 1). These tags developed are described herein. These tags, unlike many currently used, allow for the simultaneous quantitation and characterization of glycans and enhanced chemical stability.

Indeed, the compounds and methods of the present invention are capable of quantitating glycans via fluorescence capabilities and unlike many prior art methods, are capable of the characterization of glycans via induced fragmentations by mass spectrometry and free-radical chemistry. Additionally, the compounds and methods disclosed herein may be used for glycan conjugation with any glycan that has a reducing terminus. In comparison, many prior art methods are limited to N-glycans only.

The compounds of the present invention are capable of introducing a TEMPO (2,2,6,6-tetramethylpiperidine-1-oxyl) group or analog thereof to glycans and similar molecules which in turn allows for free-radical chemistry to occur. TEMPO groups are superior to other groups, such as succinimide groups in many ways. For example, succinimide groups generally quickly decomposition in the presence of atmospheric moisture while TEMPO groups generally do not.

This invention will be better understood by reference to the Experimental Details which follow, but those skilled in the art will readily appreciate that the specific experiments detailed are only illustrative of the invention as described more fully in the claims which follow thereafter.

### EXAMPLES

### Example 1

General Synthesis. As shown in Scheme 1, the general synthesis of a fluorescent tag begins with a cyclization reaction involving a substituted benzene and an olefin. The final product was obtained through a series of six reactions.

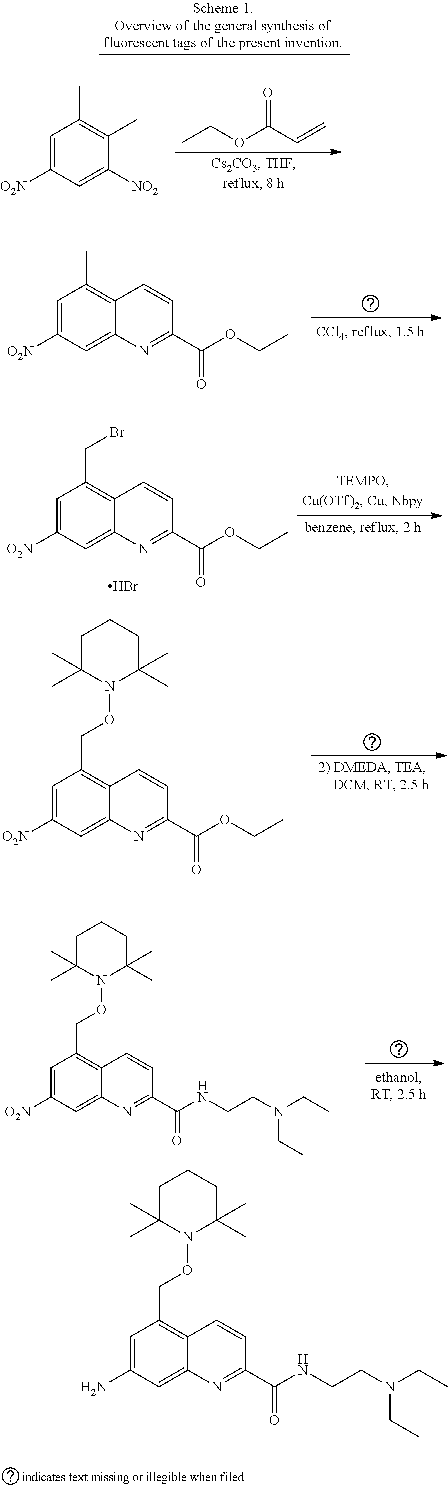

### Ethyl 5-methyl-7-nitro-2-quinolinecarboxylate (1)

In a clean, oven-baked flask equipped with a stir bar, 1,2-dimethyl-3,5-dinitrobenzene (5 mmol) was allowed to react with ethyl acrylate (2 equiv.) in a solution of cesium carbonate (3 equiv.) in anhydrous tetrahydrofuran (25 mL) under argon. After 8 h of reflux, the crude mixture was allowed to cool down and was evaporated in vacuo before the product was extracted from the residue with dichloromethane. The collections were purified via silica gel flash chromatography by using ethyl acetate and hexanes. The desired product was obtained as a yellow solid (360.5 mg, 28% yield).

### Ethyl 5-bromomethyl-7-nitro-2-quinolinecarboxylate hydrobromide (2)

A flame-dried flask was charged with ethyl 5-methyl-7-nitro-2-quinolinecarboxylate (1.385 mmol), N-bromosuccinimide (1.25 equiv.), benzoyl peroxide (0.1 equiv.), and vacuumed for 30 min before placing them under an atmosphere of argon. The reactants were dissolved in anhydrous carbon tetrachloride (70 mL) and refluxed for 1.5 h with alight catalyst (250 W) and stirring. Upon completion, the crude mixture was allowed to cool down to room temperature and was purified via silica gel flash chromatography with ethyl acetate and hexanes (3.7). The desired product was converted to a salt by adding hydrobromic acid followed by 30 min of stirring and completely evaporating the solvents under reduced pressure.

### Ethyl 5-(TEMPO)methyl-7-nitro-2-quinolinecarboxylate (3)

The ethyl 5-bromomethyl-7-nitro-2-quinolinecarboxylate hydrobromide (assuming 1.385 mmol) from the previous step was converted back to neutral form by dissolving the salt in water and basifying the solution with sodium hydroxide until pH ˜7. The organic compound was extracted with benzene (60 mL) and the collections were dried over anhydrous sodium sulfate. The dried benzene layer was transferred to a flame-dried flask and degassed with argon for 30 min after dissolving TEMPO (1.2 equiv.), Cu(OTf)₂ (0.1 equiv.), Nbpy (0.4 equiv.), and copper powder (1.2 equiv.). With stirring, the solution was refluxed for 2 h under argon. After cooling down to room temperature, the crude mixture was filtered through a short pad of silica gel and eluted with ethyl acetate. The organic layer was transferred to a separatory funnel and washed with saturated NH₄Cl, 1 M NH₄OH, and brine. The organic layer was dried over anhydrous sodium sulfate before purification via silica gel flash chromatography with ethyl acetate and hexanes (1:5). The desired product was obtained as a white to off-white solid (144.8 mg, 25% overall yield for bromination followed by TEMPO coupling).

### Ethyl 5-(TEMPO)methyl-7-nitro-2-quinolinecarboxylic acid (4)

Ethyl 5-(TEMPO)methyl-7-nitro-2-quinolinecarboxylate (0.3485 mmol) was added to a clean flask and dissolved with THF (10 mL) and methanol (10 mL). An aqueous solution of 2 M KOH (20 mL) was slowly added to the mixture. The reaction was allowed to continue with stirring at room temperature. Upon completion, the crude mixture was evaporated under reduced pressure to remove THF and methanol. An aqueous solution of 2 M HCl was added to the crude mixture until pH ˜7 before extracting with ethyl acetate five times and drying the organic layer over anhydrous sodium sulfate. The organic layer was then purified via silica gel flash chromatography with methanol and DCM (20:1). The desired product was obtained as a white solid (98.7 mg, 73% yield).

### N-[2-(Diethylamino)ethyl]-5-(TEMPO)methyl-7-nitro-2-quinolinecarboxamide (5)

To an oven-baked flask, ethyl 5-(TEMPO)methyl-7-nitro-2-quinolinecarboxyic acid (0.2543 mmol) was added and vacuumed for 30 min before being dissolved in thionyl chloride (10 mL). The reaction was allowed to occur by refluxing with stirring for 2 h. Afterwards, the thionyl chloride was evaporated in vacuo and the residue was immediately redissolved in anhydrous DCM (5 mL). In a separate oven-baked flask, N-diethylethylenediamine (50 μL) and anhydrous trimethylamine (140 μL) were dissolved in anhydrous DCM (5 mL). The acid chloride was added to this mixture in a dropwise fashion over the course of 10 min. After 2.5 h of stirring at room temperature, the mixture was diluted with 15 mL of DCM and washed with saturated aqueous sodium bicarbonate once. The aqueous layer was then extracted with DCM twice and the combined organic layers were dried over anhydrous sodium sulfate. The organic layer was then subjected to purification via silica gel flash chromatography with methanol and DCM. The desired product was obtained as a white solid (100.8 mg, 82% yield).

### 7-Amino-N-[2-(diethylamino)ethyl]-5-(TEMPO)methyl -2-quinolinecarboxamide (6)

N-[2-(Diethylamino)ethyl]-5-(TEMPO)methyl-7-nitro-2-quinolinecarboxamide (0.2076 mmol) was added to a clean flask equipped with a stir bar. The solid was dissolved in absolute ethanol (5 mL) prior to an addition of 10% Pd/C (0.5 equiv.). A source of hydrogen gas was attached to the flask and the reaction was allowed to occur at room temperature with stirring for 2.5 h. Upon completion, the crude mixture was filtered through sand and celite followed by elution with ethanol. The organic layer was purified via silica gel flash chromatography with methanol and DCM to obtain the final pure product as a yellow solid (51.5 mg, 54% yield).

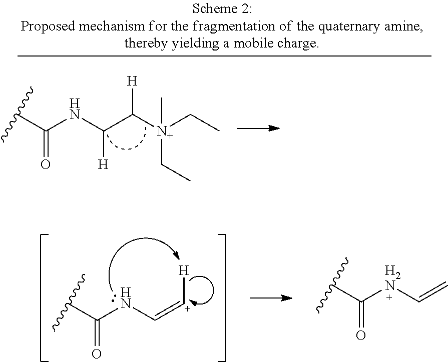

TABLE 1
The gradient for the HILIC-FLD-ESI +
MS analysis of the labeled glycans.
Mobile Phase A: 50 mM ammonium formate, pH 4.4
Mobile Phase B: LC-MS acetonitrile

| Time | Flow Rate (mL/min) | % A | % B | Curve |
|---|---|---|---|---|
| 0 | 0.4 | 14 | 86 | 4 |
| 35.0 | 0.4 | 50 | 50 | 4 |
| 36.5 | 0.2 | 100 | 0 | 6 |
| 39.5 | 0.2 | 100 | 0 | 5 |
| 43.1 | 0.2 | 14 | 86 | 6 |
| 47.6 | 0.4 | 14 | 86 | 5 |
| 55.0 | 0.4 | 14 | 86 | 5 |

TABLE 2
The peak integrations and peak masses for the HILIC-FLD-
ESI + MS analysis of the labeled maltosaccharides.

| | | Peak | | Peak Mass | Theoretical Mass | Mass Error |
|---|---|---|---|---|---|---|
| Peak | Analyte | Area | pmol | [M + H]⁺ | [M + H]⁺ | (ppm) |
| 1 | Maltose | 1.090e8 | 7.8 | 782.4545 | 782.45460 | −0.09 |
| 2 | Maltotriose | 1.044e8 | 7.5 | 944.5075 | 944.50742 | 0.09 |
| 3 | Maltotetraose | 1.026e8 | 7.4 | 1106.5604 | 1106.56025 | 0.16 |
| 4 | Maltopentaose | 1.096e8 | 7.9 | 1268.6141 | 1268.61307 | 0.84 |
| 5 | Maltohexaose | 1.050e8 | 7.6 | 1430.6661 | 1430.66589 | 0.17 |
| 6 | Maltoheptaose | 1.091e8 | 7.8 | 1592.7189 | 1592.71872 | 0.10 |

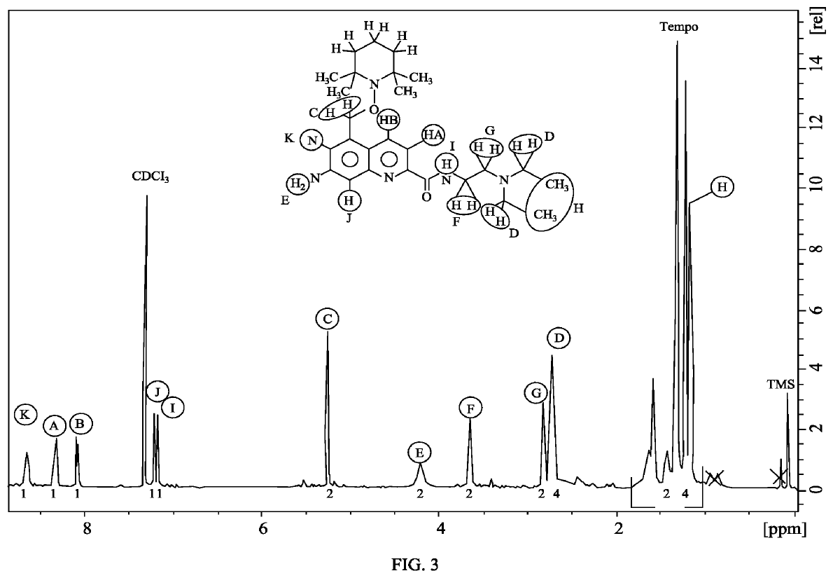

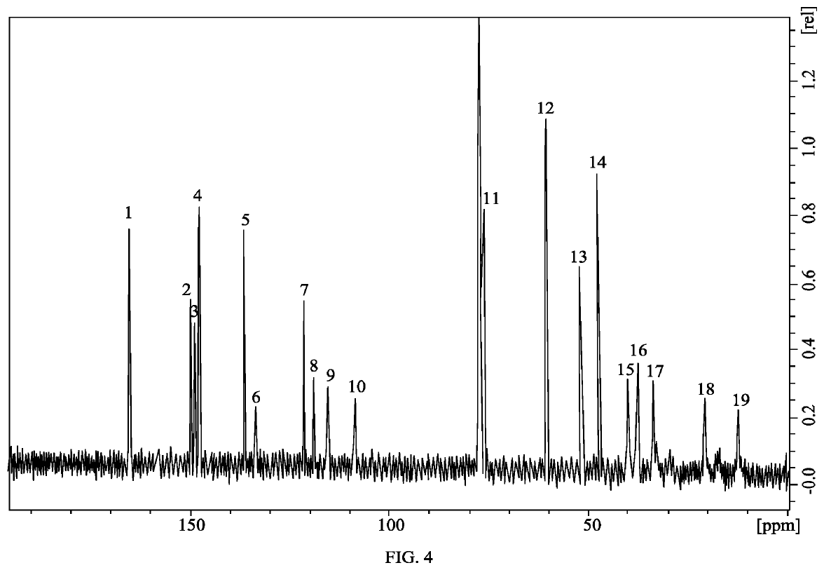

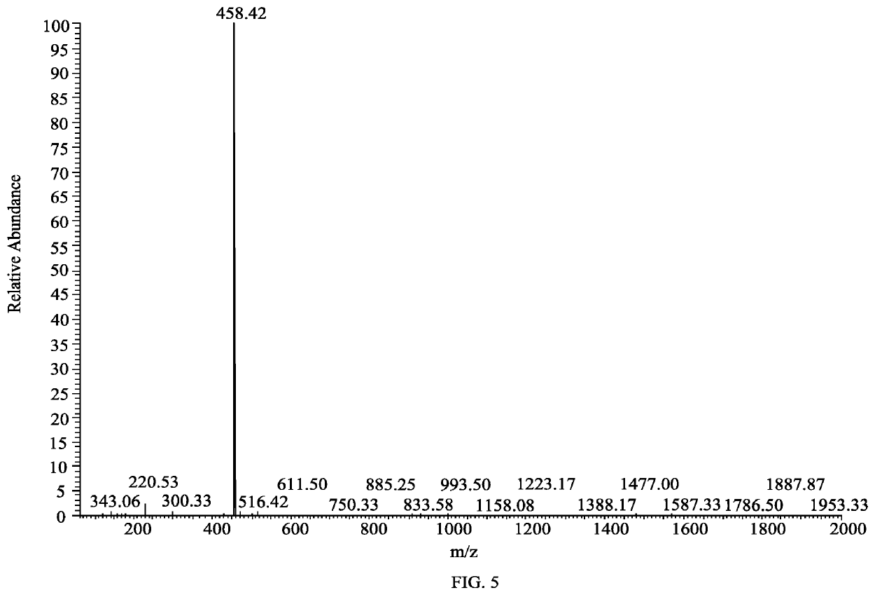

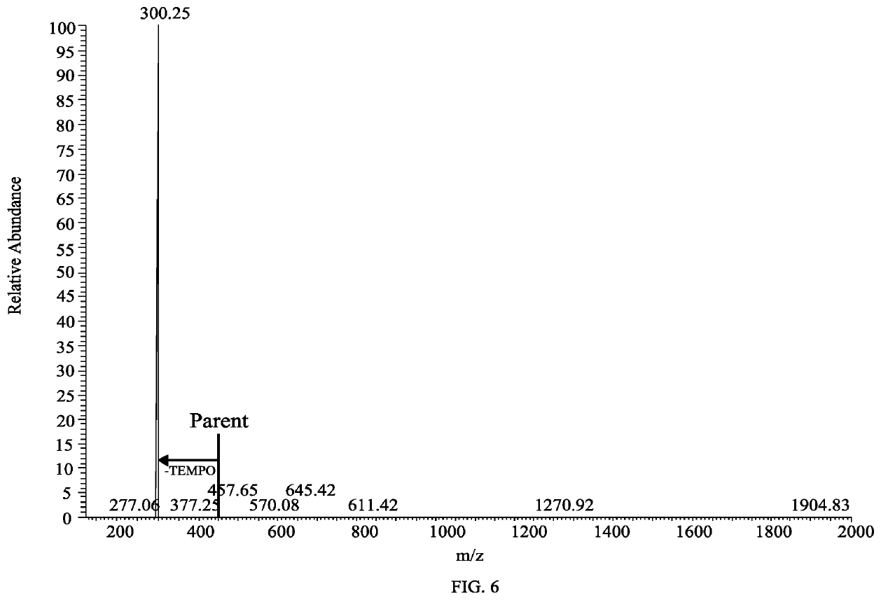

FIGS. 3-7 relate to the nuclear magnetic resonance spectrum and mass spectrometry spectrum of 7-Amino- N-[2-(diethylamino)ethyl]-5-(TEMPO)methyl -2-quinolinecarboxamide. These spectra confirm the chemical structure of this compound.

Glycan Sample Preparation with 7-Amino- N-[2-(diethylamino)ethyl]-5-(TEMPO)methyl -2-quinolinecarboxamide. A volume of 30 μL of 20 mM fluorescent tag (7-Amino- N-[2-(diethylamino)ethyl]-5-(TEMPO)methyl -2-quinolinecarboxamide) in methanol and 20 L of 1 mM glycan sample (e.g., lactose) in water were added into a PCR tube together and evaporated in vacuo. The residue was redissolved in 50 mM sodium cyanoborohydride in DMSO and acetic acid (7:3 v/v). The mixture was incubated at 70° C. for 2 h. After completion of the glycan labeling, the mixture was evaporated in vacuo at 70° C. The residue was cleaned with HPLC-grade acetone to extract the unreacted reagent and sodium cyanoborohydride. The precipitate was dissolved in 50% HPLC-grade methanol in 18.2 megaohm water prior to analysis via LC-MS.

Analysis via Mass Spectrometry. A Thermo-Fisher Scientific™ linear quadrupole ion trap (LTQ-XL) mass spectrometer (Thermo, San Jose, CA, USA) equipped with an electrospray ionization (ESI) was utilized. The samples were directly infused into the ESI source of the mass spectrometer at a flow rate of 10 μL/min. The parameters of the mass spectrometer included a sheath nitrogen gas flow rate of 10 (arbitrary units), a spray voltage of 5.00 kV, a capillary voltage of 20-40 V, a capillary temperature of 275° C., and a tube lens voltage of 50-200 V. The signal intensities were maximized by optimizing the ion optic parameters via the auto-tune function within the LTQ-XL tune program. Systematic glycan fragmentations were accomplished by subjecting the ionized bioconjugates to collisional-induced dissociation (CID) of 15 to 50 (arbitrary units).

Fluorescence Assays. A concentration range of 1 pM to 100 μM was prepared to assess the fluorescence capabilities of the fluorescent tag. An absorbance scan was first performed on the 100 μM solution to determine the potential excitation wavelengths. Each excitation wavelength was then tested for emission via the Agilence Cary Eclipse fluorescence spectrophotometer. The optimal excitation and emission wavelengths were selected for the construction of a preliminary calibration curve to assess for any correlation between fluorescence and concentration. The preliminary calibration curve was also constructed on another instrument (ThermoFisher Scientific™ NanoDrop™) for instrument reproducibility.

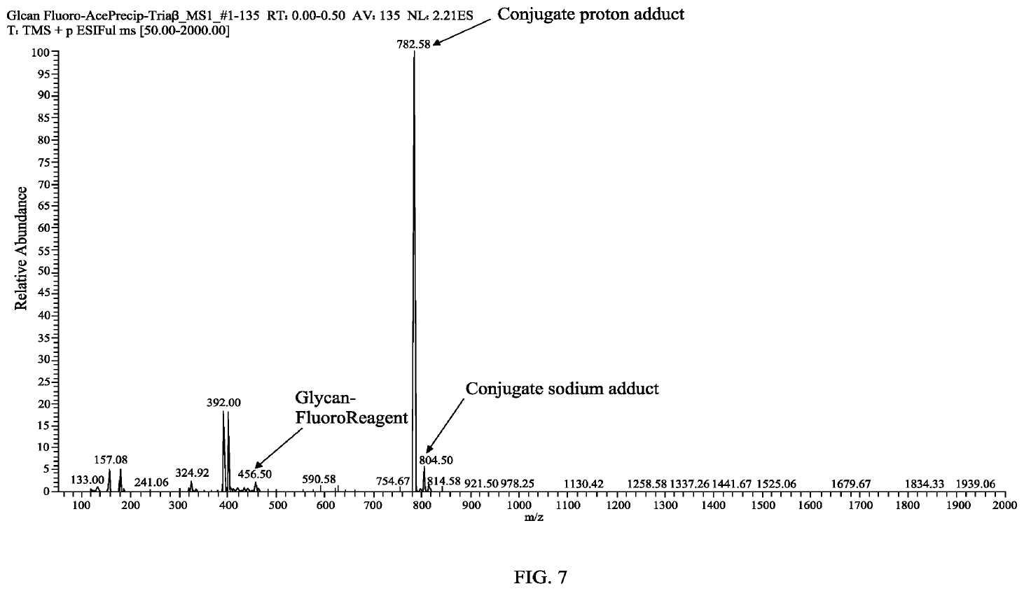

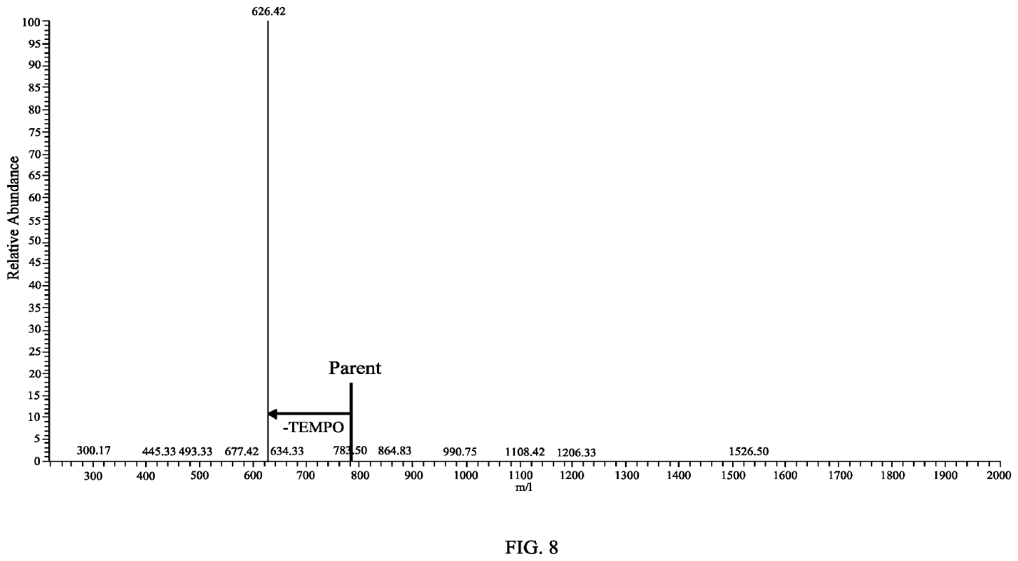

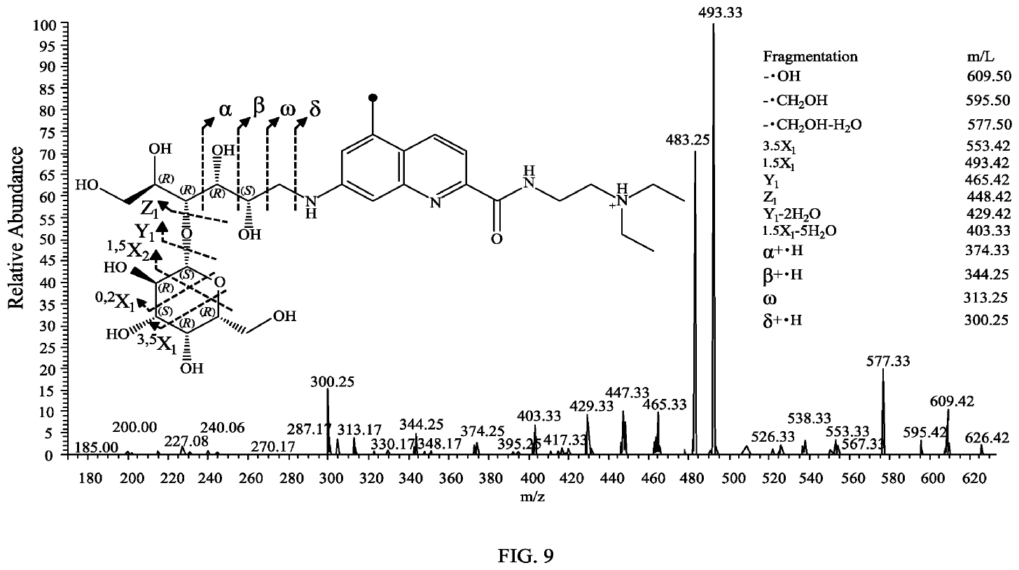

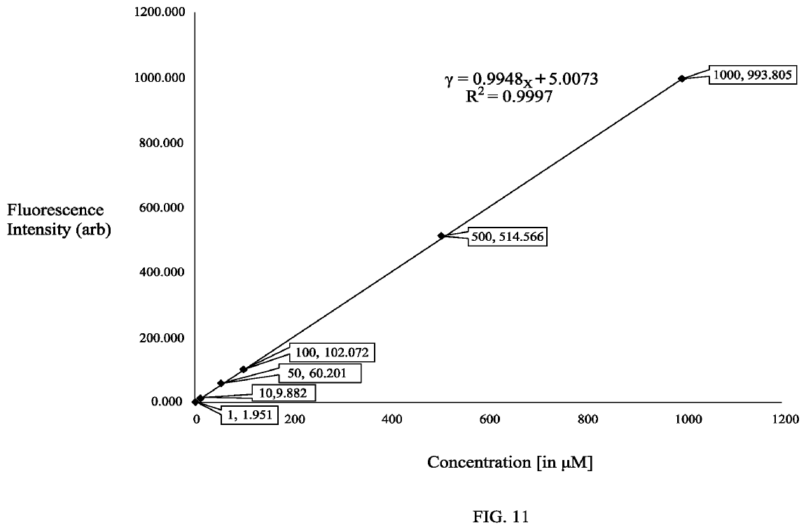

FIGS. 7-11 provide the results of this experiment, which involved the labeling of a disaccharide sample. This experiment was performed involving the labeling of a disaccharide sample of known molecular weight and chemical structure by using the invention and established protocols. The labeling was successful given the expected increase in the m z values of the invention (m/z 456) to the labeled glycan (m/z 782) via mass spectrometry. The labeled disaccharide was then isolated, and fragmentations were induced. The first fragmentation that occurred was the expected loss of TEMPO, which yields a loss of m/z 156. This initial fragmentation verified that the glycan had been labeled with the compound 7-Amino- N-[2-(diethylamino)ethyl]-5-(TEMPO)methyl -2-quinolinecarboxamide. A further isolation and fragmentation of the fragment (the isolated fragment has an m z of 626) revealed additional fragmentations involving the covalently attached disaccharide. These fragmentations can be used to characterize the disaccharide.

UPLC-FLD and UPLC-MS Analysis. A ThermoFisher Scientific™ Vanquish Flex™ UPLC instrument equipped with a fluorescence detector and a ThermoFisher Scientific™ Q Exactive Plus Oribtrap mass spectrometer were utilized for all of the following analyses. A mixture of six reagent-labeled glycans (maltose, maltotriose, maltotetraose, maltopentaose, maltohexaose, and maltoheptaose) were each prepared to a final concentration within the range of 10-20 μM. A Supelco Discovery C₁₈ column (5 μm particle size, L 25 cm×I.D. 4.6 mm) and an XBridge Glycan BEH Amide XP column (2.5 μm particle size, L 150 mm×I.D. 3.0 mm) were used for the separation of the analytes. The flow rate was set to 0.750 mL/min and 0.400 mL/min with a column temperature of 30.00° C. and 60.00° C. for the C₁₈ column and the Glycan BEH Amide column, respectively. For the C₁₈ column, mobile phase A consisted of 99.9% 18.2-MΩ water and 0.1% formic acid and mobile phase B consisted of 99.9% LC-MS grade acetonitrile and 0.1% formic acid. The gradient was held at 10% B for 5 min and was then increased to 30% B over the course of 55 min. For the Glycan BEH Amide column, mobile phase A consisted of an aqueous solution of 50 mM ammonium formate, pH 4.403, and mobile phase B consisted of LC-MS grade acetonitrile. The gradient started at 86% B and reduced to 50% B over the course of 55 minutes. The fluorescence detection took place with the parameters set to an excitation of 385 nm, an emission of 515 nm, and detection sensitivity of 5. The mass spectrometric detection took place with the parameters set to a capillary voltage of +3.50 kV, a capillary temperature of 320° C., sheath gas flow rate of 50 (arb), and an S-lens RF level of 50.0 (arb).

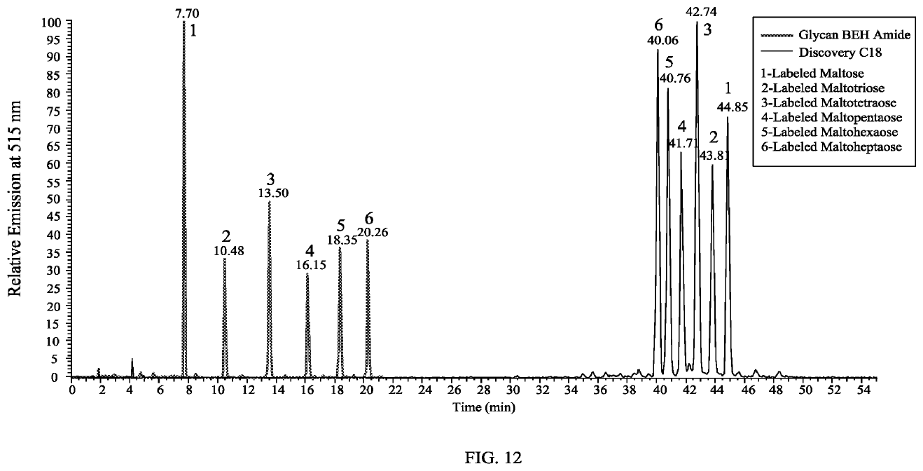

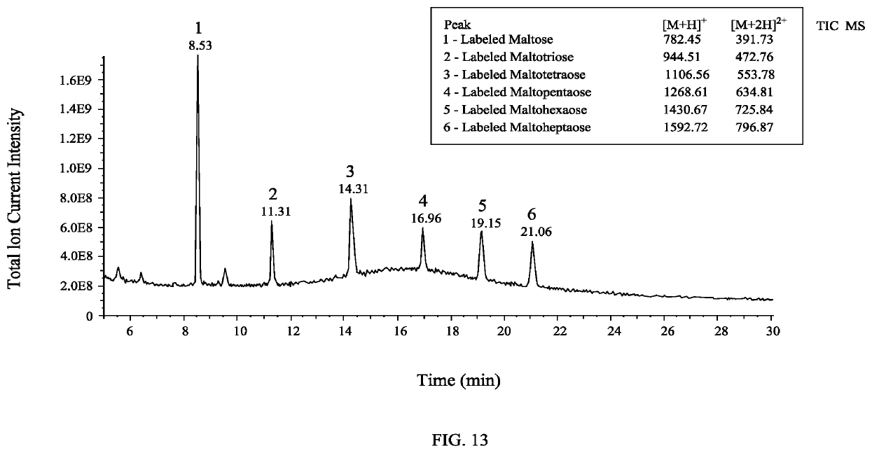

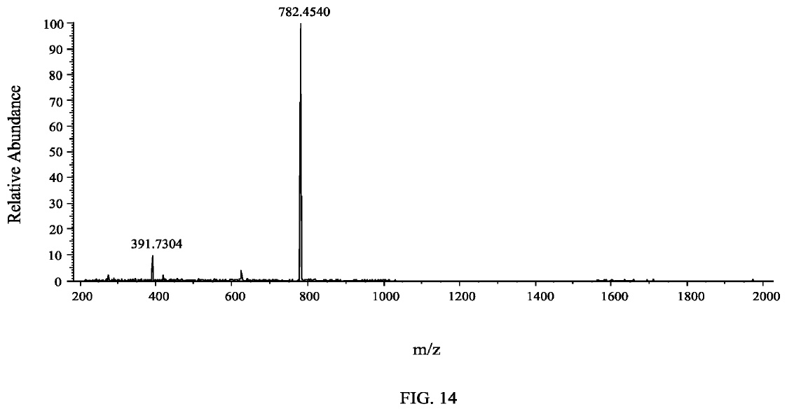

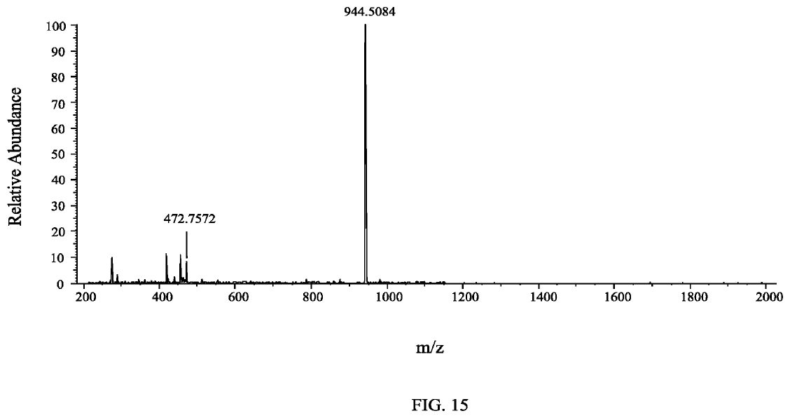

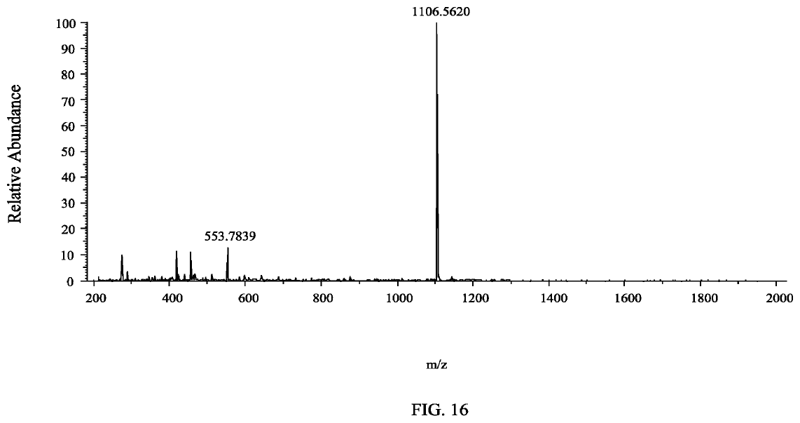

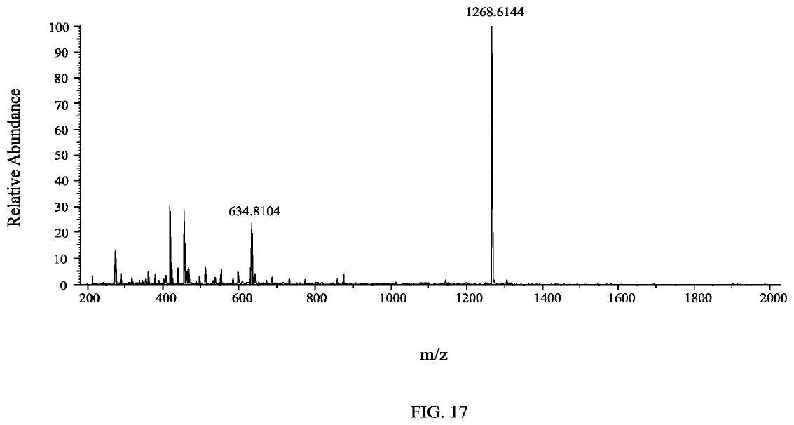

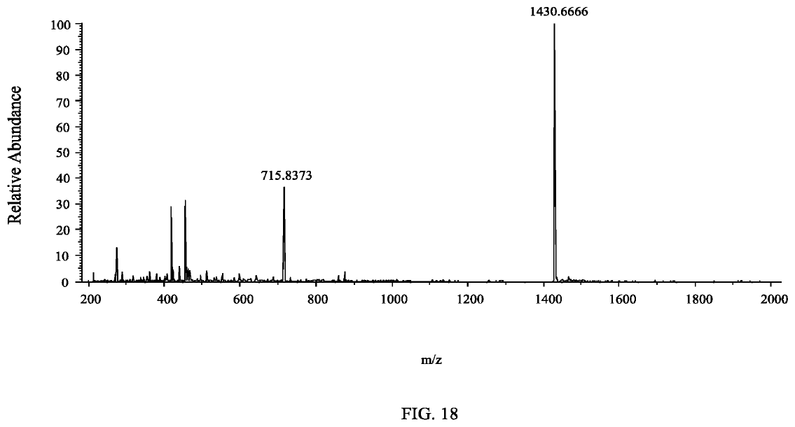

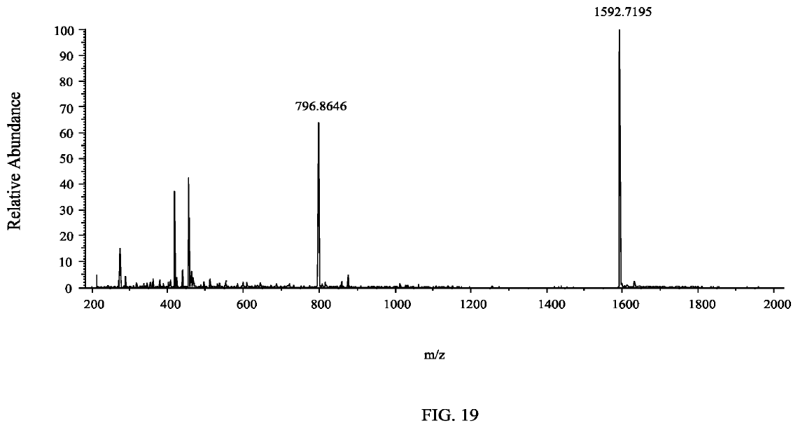

The results for this experiment are shown in FIGS. 12-19 which includes fluorescence experimental data. The excitations wavelengths of 270 nm and 390 nm were assessed for any light emission. Both excitation wavelengths led to an emission at a wavelength of 520 nm. Further experiments and discussions revealed that 390 nm is the better excitation wavelength for use in fluorescence assays. Calibration curves were then successfully constructed on two different fluorimeters with a linear fit of 0.9997 between the nanomolar to micromolar concentrations of the invention.

### Conclusion

For optimal mass spectrometric and liquid chromatographic analysis, and fluorescence detection, of glycans, the herein disclosed fluorescent reagents contain important chemical sites. Among these sites, as mentioned previously, include the fluorophore base, basic site (the ionization/methylation site), glycan labeling site (the coupling site), and the free radical precursor, which all commercially-available tags lack. The fluorophore base exhibits the fluorescence capabilities required for detection and quantitation abilities of glycans in a concentration-dependent manner. The purpose of utilizing fluorescence is to maximize the sensitivity of liquid chromatographic detection and quantitation methods. The basic site allows for the induction of a positive charge via either protonation, ion-dipole interactions, or methylation. The importance behind the recruitment of the basic site is to allow for ionization to take place during mass spectrometric analysis of glycans. The glycan labeling site is the most essential feature to include on the tag as it allows for a reaction with the glycans, which subsequently forms a covalent attachment. Lastly, the free radical precursor allows for the formation of a nascent free radical during any of the dissociation techniques mentioned earlier. Such dissociation techniques include collisional-induced dissociation (CID) and higher-energy collision dissociation (HCD). Ultimately, the free radical simultaneously reacts with the glycan to yield glycan fragmentations that are reproducible, systematic, and efficient towards glycan characterization, identification, and differentiation among glycans and their isomers.

Based on the data presented, the fluorescent tags disclosed herein exhibit an excellent correlation between fluorescence and concentration from as low as 1 nM. This correlation was also observed on another fluorescence spectrophotometer instrument with similar results. Moreover, the basic site on the fluorescent tag is efficient towards ionization processes during mass spectrometric analysis. The fluorescent tag, upon tagging of glycan molecules, is capable of inducing systematic and reproducible fragmentations for glycan analysis. Thus, the enlistment of the fluorophore base, basic site, glycan labeling site, and the free radical precursor, are altogether an optimal combination towards glycan tagging and analysis. With the use of liquid chromatography for glycan and glycan isomer separation paired with fluorescence detection and tandem mass spectrometry for glycan and glycan isomer fragmentations, and thus, characterization and identification, the fluorescent tag will ultimately provide powerful, novel, and modern analytical capabilities within the field of glycomics. As such, the evolvement of novel analytical techniques allows for the growth of a relatively new field which has the potential for the study of glycans as human disease biomarkers.

### Example 2

Materials. For the synthesis of the novel fluorescent tag, the starting material 1,2-dimethyl-3,5-dinitrobenzene was purchased from 1ClickChemistry (Kendall Park, NJ). All other chemicals that were used for the synthesis of the novel fluorescent tag were purchased from Sigma-Aldrich (St. Louis, MO, USA). Maltose, maltohexaose, maltoheptaose, and GPC-grade dextran ladder standards were purchased from Sigma-Aldrich (St. Louis, MO, USA). Maltotriose was purchased from Thermo Scientific (Waltham, MA). Maltotetraose and maltopentaose were acquired from Biosynth Carbosynth (Staad, Switzerland). Lacto-N-difucohexaose I (LNDFH I) and lacto-N-difucohexaose II (LNDFH II) were purchased from Dextra Laboratories (Reading, UK). Bovine pancreas ribonuclease B (RNase B) and peptide-N-glycosidase F (PNGase F) were purchased from New England Biolabs (Ipswich, MA, USA). LC-MS grade ammonium formate was purchased from Sigma-Aldrich (St. Louis, MO, USA). All solvents that were used for the purification and analysis of the samples described herein were of HPLC-grade and purchased from Fisher Scientific (Hampton, NH).

N-Glycan Deglycosylation of Rnase B. The manufacturer's protocol was followed for the deglycosylation ofN-glycans from RNase B. To ensure full deglycosylation of the glycoprotein, the incubation step was elongated to 14 h. Afterwards, the resulting aqueous solution was cooled down to room temperature and then subjected to purification by PGC SPE. The cartridge was activated with acetonitrile and equilibrated with 5% acetonitrile prior to sample application. Afterwards, the cartridge was washed with 1 mL of water for a total of five times and the glycans were eluted and collected through a 0.2 μm nylon filter with 250 μL of 40% ACN with 0.1% formic acid for a total of four times. The eluate was evaporated in vacuo at 60° C. prior to following the derivatization step.

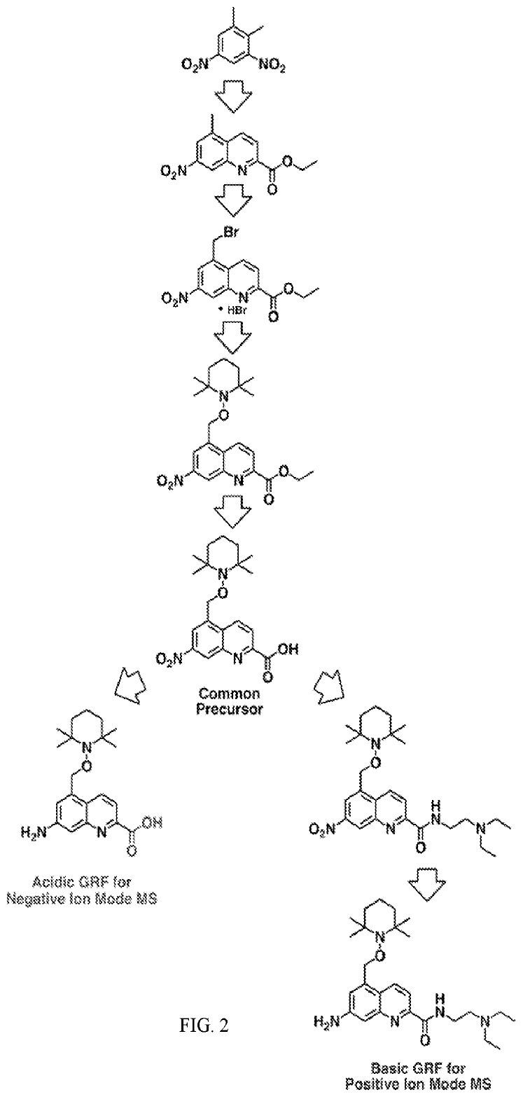

Synthesis of 7-Amino-N-[2-(diethylamino)ethyl]-5-(TEMPO)methyl-2-quinolinecarboxamide. Briefly, 1,2-dimethyl-3,5-dinitrobenzene underwent a cyclization reaction with ethyl acrylate. Following this, the remaining methyl group was brominated, and the product was converted to the hydrobromide salt form. After neutralization and extraction into the organic solvent that was used in the subsequent reaction, the intermediate underwent a TEMPO coupling reaction. The resulting product was then subjected to strong base to yield a carboxylic acid (referred to as the common precursor as shown in FIG. 2). The carboxylic acid was first converted to an acid chloride intermediate prior to reaction with N,N-diethylethylenediamine via an amide bond formation. Lastly, the remaining nitro group is hydrogenated with hydrogen gas to yield the final product. Alternatively, an acidic analog of this structure for analyses of glycans by negative ion mode mass spectrometry (currently under investigation) can be obtained by directly hydrogenating the nitro group from the common precursor mentioned previously.

N-Glycan and Standardized Glycan Sample Preparation with 7-Amino-N-[2-(diethylamino)ethyl]-5-(TEMPO)methyl-2-quinolinecarboxamide. A volume of 30 μL of 20 mM of the tagging reagent in methanol and 20 μL of 1 mM of the standardized glycan in water were added together and evaporated in vacuo. The residue was redissolved in 50 mM sodium cyanoborohydride in anhydrous DMSO and glacial acetic acid (7:3 v/v) and the resulting mixture was incubated at 70° C. for 2 h. After completion of the glycan labeling, the mixture was evaporated via nitrogen degassing or evaporated in vacuo at 70° C. For a total of three repetitions, the residue was vortexed and sonicated with 50 μL of HPLC-grade acetone to extract the unreacted reagent and sodium cyanoborohydride, centrifuged for 5 min at 14 krpm, and the supernatant containing the free reagent was recycled for future desalting and purification. After the final collection, the pellet containing the tagged glycans was allowed to completely dry under ambient conditions for 10 min prior to LC-MS analysis.

Fluorescence Assays. A concentration range of 1 nM to 100 μM was prepared to assess the linearity of the fluorescent tag. An excitation scan with the emission mode set to zero order was initially performed via the Agilence Cary Eclipse fluorescence spectrophotometer on a 100 μM solution to determine the preliminary excitation wavelength values. Each excitation wavelength was then subjected to an emission scan to determine the wavelength pair with the optimal fluorescence intensity. With the parameters set to a 965 V PMT detector voltage, 5 nm excitation and emission slits, and 1.000 s averaging time, the optimal excitation and emission wavelengths at 280 nm and 520 nm, respectively, were selected for the construction of a concentration-dependent fluorescent plot to assess the correlation between fluorescence intensity and concentration.

UPLC-FLD and UPLC-MS Analysis. A ThermoFisher Scientific Vanquish Flex UPLC instrument equipped with a fluorescence detector and a ThermoFisher Scientific Q Exactive Plus Oribtrap mass spectrometer were utilized for all of the following experiments. A mixture of six maltosaccharides consisting of maltose, maltotriose, maltotetraose, maltopentaose, maltohexaose, and maltoheptaose were labeled and diluted to make a 46 pmol/μL solution. The labeled dextran ladder was prepared to a final concentration of 300 μM. The labeled LNDFH I and LNDFH II analytes were mixed in unknown quantities with an unknown total concentration. For the analysis of N-glycans from RNase B, a concentration of 74 pmol/μL of tagged N-glycans was prepared. The labeled glycans were reconstituted in the initial state mobile phase consisting of 50 mM ammonium formate, pH 4.4 and LC-MS acetonitrile (14:86 v/v) and injections were made at 1.0 μL. An Xbridge Glycan BEH Amide XP column (2.5 μm particle size, L 150 mm×I.D. 3.0 mm) was utilized for the separation of the fluorescent tag-derivatized glycan analytes according to the gradients described in Table 1. The flow rate was set to 0.400 mL/min with a column temperature of 60.00° C. The fluorescence detection took place with the parameters set to an excitation of 280.0 nm, an emission of 520.0 nm, detection sensitivity of 8 (arb), and a scan rate of 5.00 Hz. The mass spectrometric experiments were conducted with the parameters set to a capillary voltage of +3.50 kV, a capillary temperature of 263° C., sheath gas flow rate of 50 (arb), auxiliary gas heater temperature of 425° C., auxiliary gas flow rate of 13 (arb), sweep gas flow rate of 3 (arb), and an S-lens RF level of 50.0 (arb). For the MS² analysis of the analytes during each run, an isolation window of 4.0 m/z was implemented for each of the expected masses listed in the inclusion list at a resolution of 70,000. The HCD collisional energy was varied under normalized collision energy (NCE) mode.

### Results and Discussion

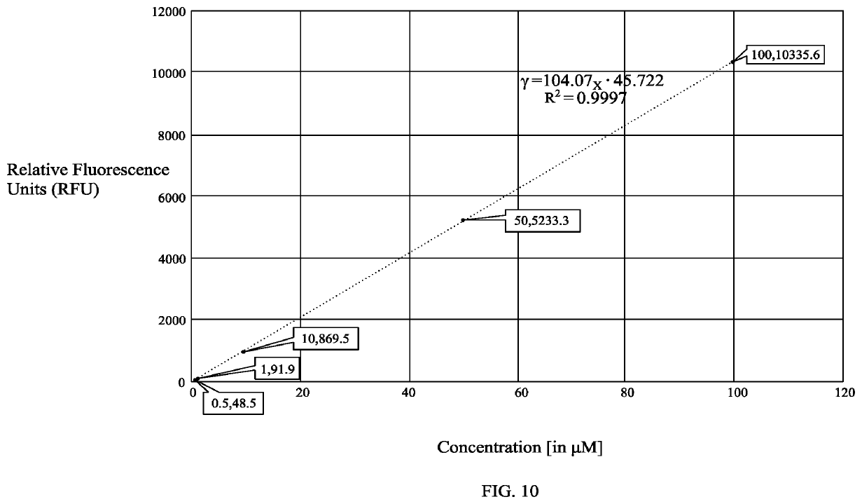

A novel fluorescent tag that enlists a free radical precursor (Glyc•RadiFluor; abbreviated as GRF) was synthesized to enhance the glycan characterization capabilities, in addition to facilitating the high fluorescence sensitivity, feasible methylation and mass spectrometric ionization, and improved chemical stability, while also eliminating the interconversion between multiple isomers at the glycan reducing end. The fluorescence detection and quantitation capabilities of the GRF were first assessed prior to the execution of additional experiments involving glycans. After determining the optimal excitation and emission wavelengths as 280±5 nm and 520±5 nm, respectively, the linearity was further assessed. The correlation between the fluorescence intensity and the GRF concentration within the range of 1 nM and 1 μM were plotted and subjected to linear regression analysis (FIG. 10). The linear fit value of 0.9998 is interpreted as an excellent linearity between fluorescence intensity and the GRF concentration. Moreover, the linearity throughout the nanomolar concentrations portrays the fluorescence detection and quantitation sensitivity of the GRF which would be suitable for the quantitative analysis of glycans derived from glycoproteins.

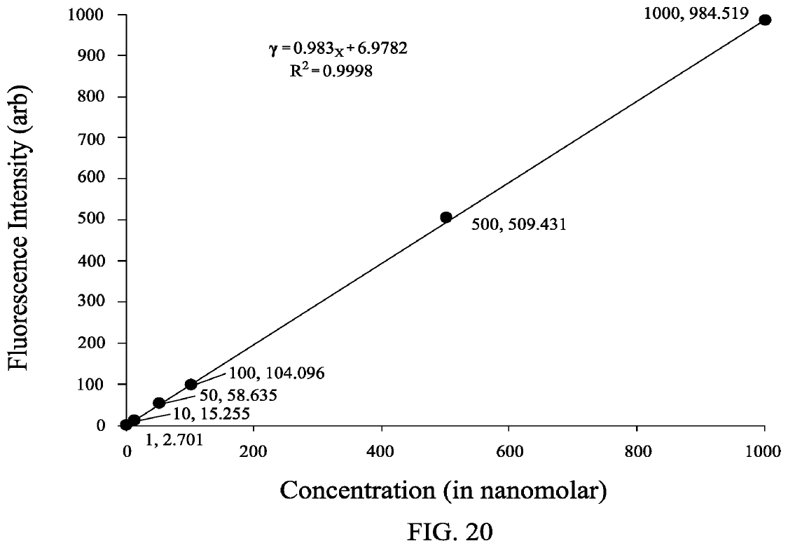

After confirming the fluorescence detection and quantitation capabilities of the GRF, a mixture of maltosaccharides of variable saccharide unit lengths were tagged via reductive amination and the separation was assessed on two columns exhibiting opposite separation methodology. Briefly, the importance of utilizing reductive amination for the tagging protocol is to eliminate the interconversion of α, β, and open-chain isomers, which would otherwise complicate LC analysis by giving rise to additional peaks. Moreover, experiments have showed that the tagging ability of the GRF during reductive amination resulted in insignificant differences in reaction yield when reacting for two hours versus 14 hours. The columns that were initially used for the separation of analytes involved both a C₁₈ stationary phase (data not shown) and an ethylene-bridged hybrid (BEH) amide (or HILIC) stationary phase, of which utilizes reversed-phase separation and normal-phase separation technology, respectively. As expected, the peak separation between the maltosaccharides was optimal when utilizing the normal-phase separation (FIG. 20). The improved separation by the BEH amide column can be explained by the increased interactions between the maltosaccharide moiety of the analyte and the hydrophilic amide groups from the stationary phase. Conversely, the hydrophobic Cis stationary phase has a reduced number of interactions with the maltosaccharide moiety. Rather, the interactions are more intimate with the fluorescent tag moiety, which explains the poorer separation of the maltosaccharides. Consequently, due to the improved peak separation, the BEH amide column was utilized for all of the future experiments described herein.

During the earlier attempts of the maltosaccharide mixture quantitative analysis, it was determined that the integration values of the peaks were not relatively equal. It was observed that the tagged maltohexaose and tagged maltoheptaose had a lower integration value relative to the shorter maltosaccharides. This problem was solved by noting the purity of the standardized glycans of longer length were relatively lower than that of the standardized glycans of shorter length. For instance, the purity of maltoheptaose and maltohexaose were 65% or greater, while the purity of maltotetraose was 95% or greater. In response, the concentrations of maltoheptaose and maltohexaose were increased during sample preparation to compensate for the reduced purity. After subsequent quantitative analysis, it was observed that the integration of the peaks in FIG. 20 returned values that were relatively equal. For the longer maltosaccharides, the width of the peaks increased slightly, thus resulting in shorter peak heights. Nonetheless, the integration of the peaks is relatively similar to those that are associated with the early-eluting maltosaccharides. Following the peak integration, the quantity of each analyte was determined based on the known total injected quantity of 46 pmol. By multiplying the ratio of the analyte peak area to the summation of all the analyte peak areas by the known total mole quantity, the result returns a value that is associated with the mole quantity belonging to the individual analyte that was injected. For instance, given that maltose has a peak area of 1.090×10⁸ and that the total combined peak area of all of the analytes is 6.397×10⁸, it can be deduced that 7.8 pmol of the GRF-derivatized maltose was injected. Similar calculations can be made for all of the other analytes that originated from the same sample (see Table 2). Based on the results of these calculations, the average analyte injection was determined to be 7.7±0.2 pmol, which agrees with the equimolar quantity of 7.7 pmol per analyte that is expected. Moreover, the normalization level of 2.43×10⁷ for a ˜7.7 pmol quantity per analyte further portrays the high detection and quantitation sensitivity of the glycans via the quinolinyl fluorophore emission upon excitation. Lastly, each maltosaccharide yields a single peak that is linked to the open-chain isomer rather than multiple peaks that correspond with the additional α and β isomers. This represents the efficiency of the reducing agent to reduce the resulting Schiff base during the labeling reaction, thereby yielding a single isomer that can no longer interconvert at the glycan reducing end. Consequently, this gives rise to a single peak for each analyte, thus establishing a simpler and more feasible analytical approach via LC towards the detection of the analytes and their counterpart isomers within any given glycan sample.

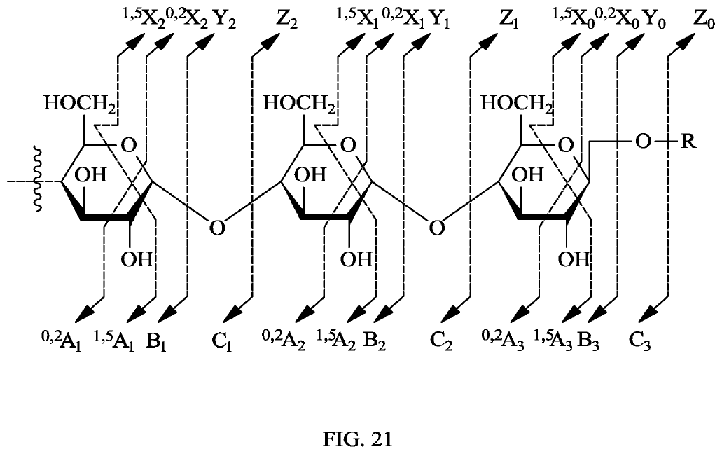

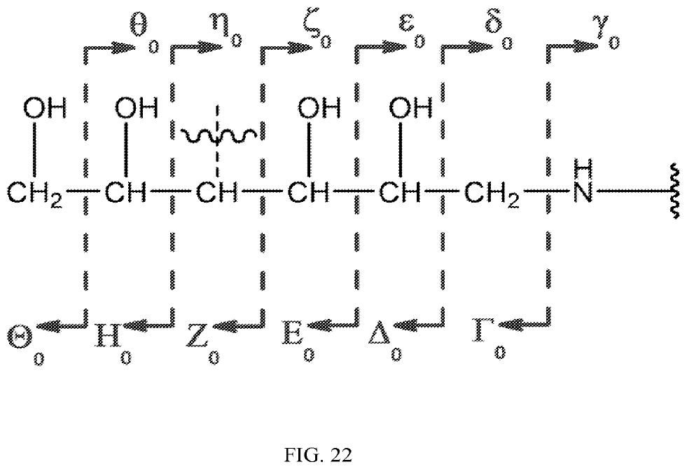

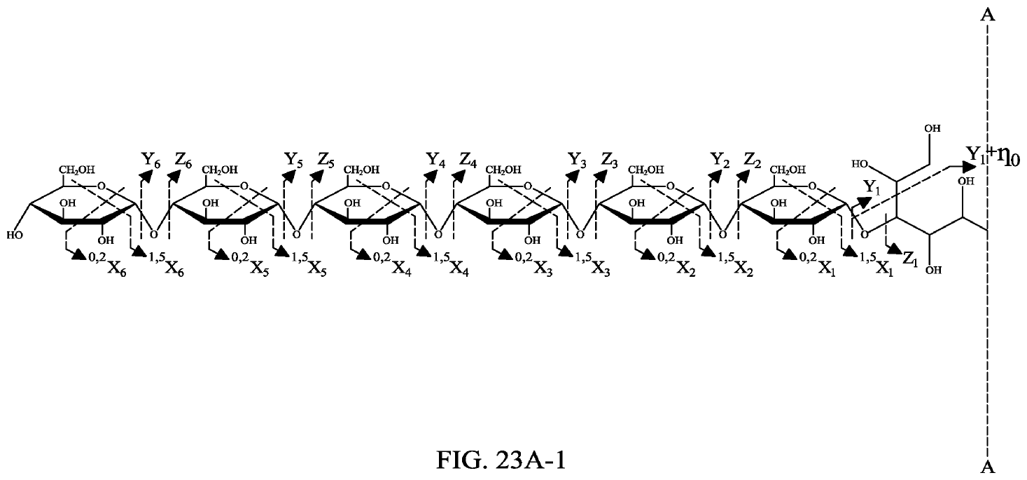

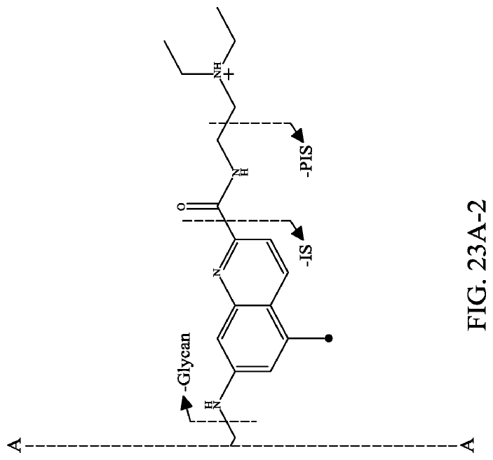

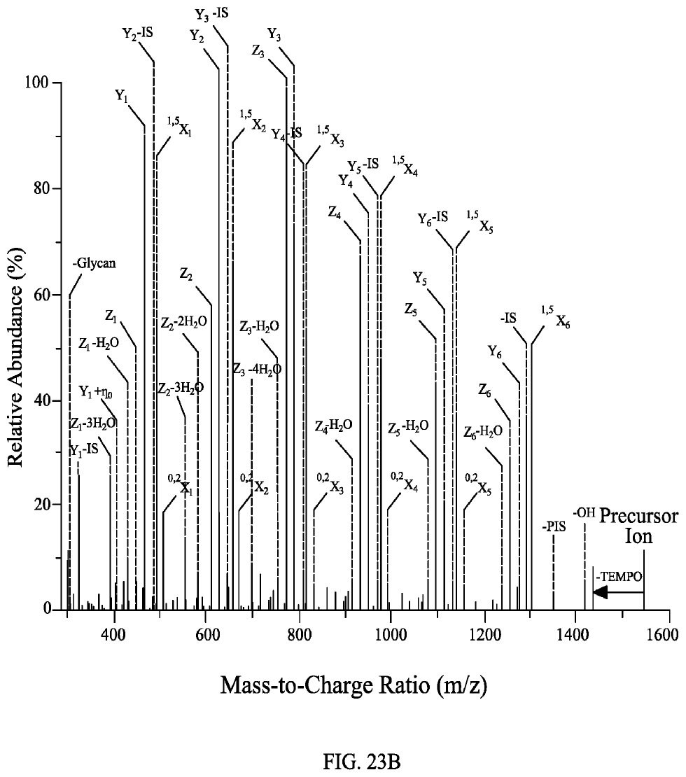

After detecting and quantitating the unknown glycans, it is especially essential to also characterize and identify them. In this example, the ability of the GRF to characterize the maltosaccharide mixture was assessed by subjecting each of the six analytes to higher-energy collision dissociation (HCD). As the HCD collisional energy is ramped up, the TEMPO free radical precursor fragments to induce a nascent, yet localized, free radical that simultaneously reacts with the glycan to result in cross-ring and glycosidic bond cleavages. Not only does the abundance of these fragmentations differ based on the structure or stereochemistry, but so does the type of fragmentations. As a result, valuable structural information can be gained through a means of different fragmentation pathways to identify and characterize a plethora of glycans and their isomers. All of the product fragmentation ions described herein are assigned based on the Domon and Costello nomenclature for glycan fragmentations (FIG. 21). Currently, no known nomenclature exists for the fragmentation ions corresponding to the reducing end open-chain isomer (saccharide unit 0). Therefore, a novel nomenclature system was accordingly devised (FIG. 22). As shown in FIGS. 23A and 23B, the singly-protonated GRF-derivatized maltoheptaose was subjected to HCD normalized collisional energy of 30 (arb) to fragment the TEMPO group and generate the free radical. Simultaneously, the highly unstable free radical reacts and induces cross-ring and glycosidic bond cleavages at each constituent saccharide unit. Among these fragmentations include the ^{0,2}X- and ^{1,5}X-type ions, which are cross-ring fragmentations, and the Y- and Z-type ions, which are glycosidic bond cleavages. An additional fragmentation of the C₂H₅O₂ group (assigned as the η₀ fragmentation), which is attached to the only tertiary carbon at the reducing end, is also observed. Additionally, multiple losses of water are observed and are associated with the Z-type fragment ions, thereby subsequently resulting in the formation of the double bonds along the C—C bonds. Although the fragmentations observed with the protonated species provide valuable information regarding the tagged maltoheptaose, numerous fragmentations can be acid-catalyzed by a mobile charge and glycan rearrangements can take place. Such glycan rearrangements result in misleading fragmentations that deviate from the fragmentations that correspond to the actual structure. The full fragmentation of the ionization site appears to catalyzed by the nascent free radical, which ultimately leads to an abstraction of a proton from the glycan moiety by the quinolinyl nitrogen. As there are multiple potential sites for protonation, the positive charge may have been previously stabilized during ionization on either the quinolinyl nitrogen or the amide nitrogen.

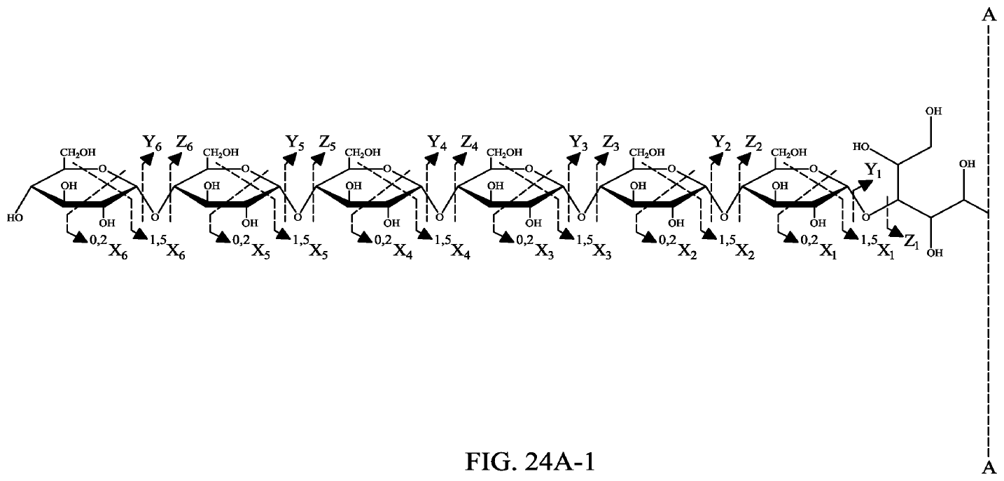

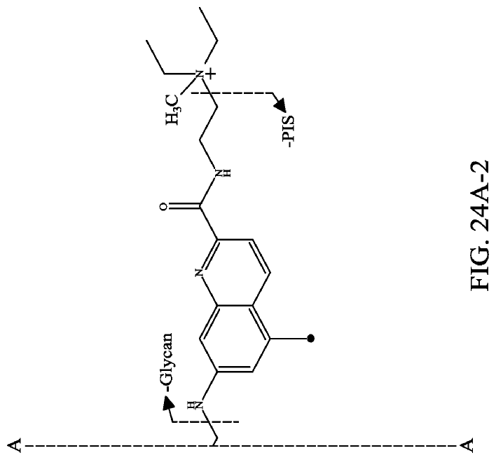

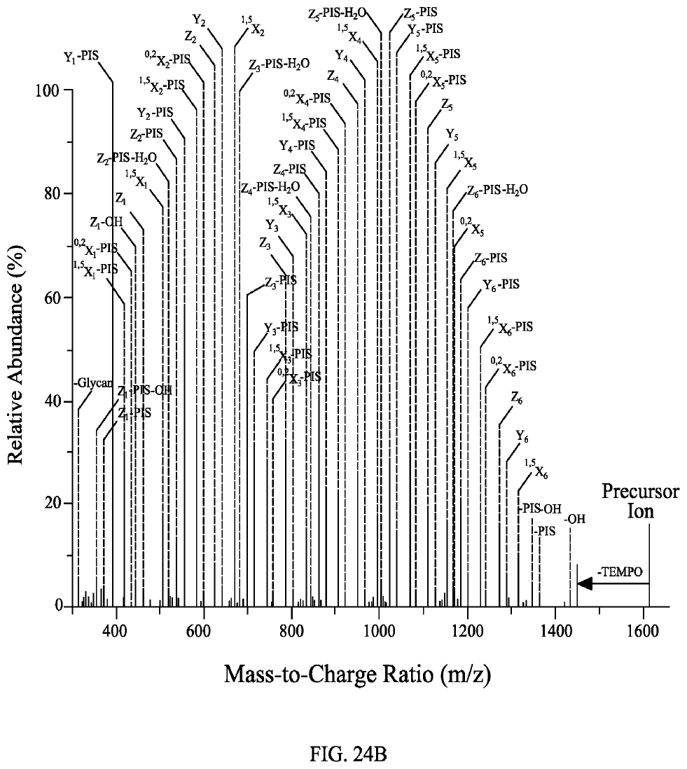

To eliminate any glycan rearrangements and acid-catalyzed fragmentations, a fixed charge is introduced by reacting the GRF-derivatized maltoheptaose with iodomethane. The tertiary amine has great affinity for nucleophiles and is selectively attacked by the methyl cation to produce a positively-charged quaternary amine. As shown in FIGS. 24A and 24B, the full loss of the ionization site is no longer observed. Although, there are additional fragmentations that are accompanied by the partial loss of the ionization site, the ^{0,2}X, ^{1,5}X, Y, and Z product ions are still observed throughout the linear biopolymer.

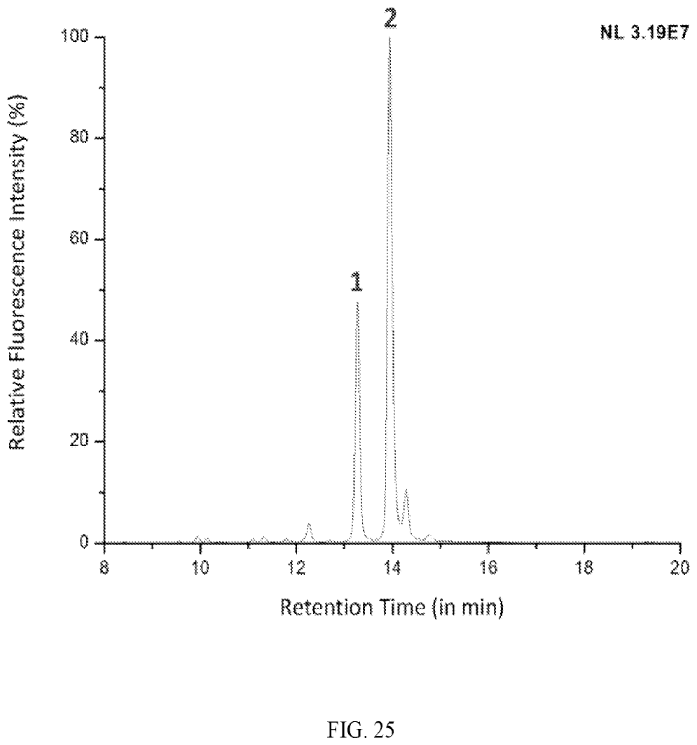

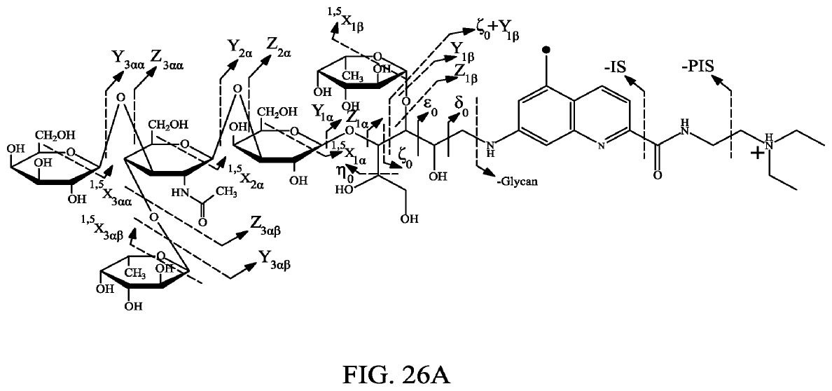

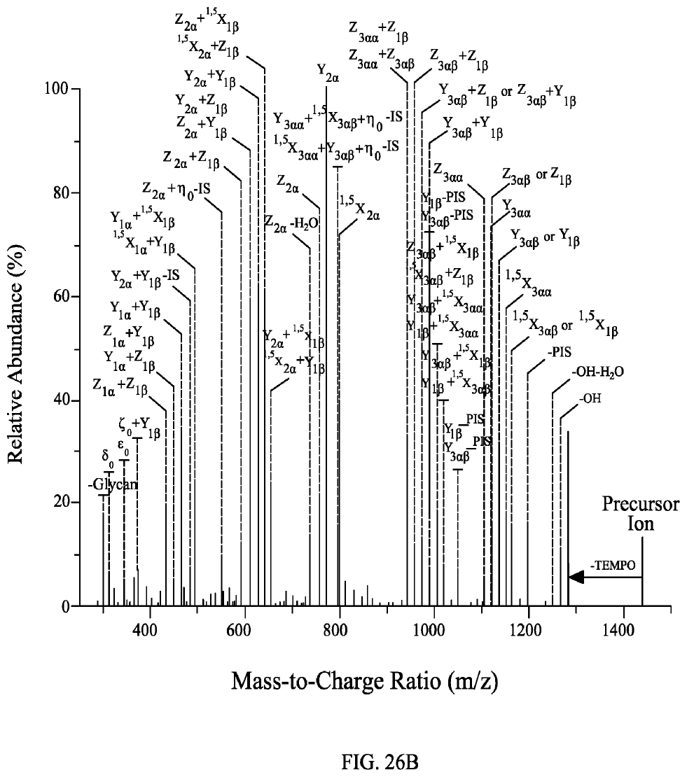

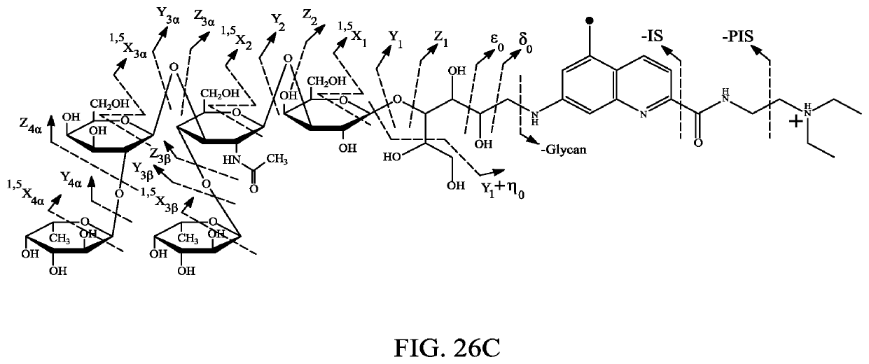

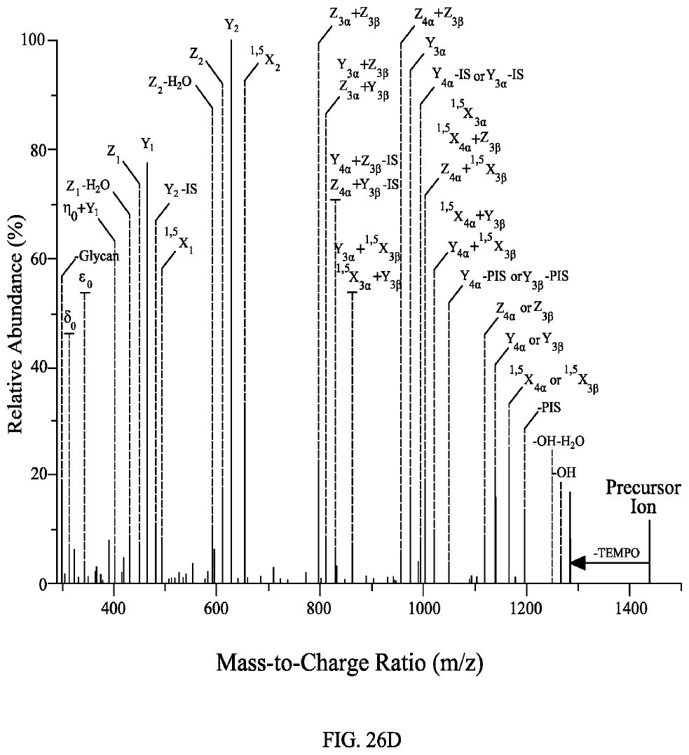

After determining the optimal method conditions and instrumentation parameters for peak separation and mass spectrometric characterization of maltosaccharides, the method was further evaluated on a mixture of branched isobaric isomers. Lacto-N-difucohexaose I (LNDFH I) and lacto-N-difucohexaose II (LNDFH II) are hexasaccharide isomers that differ only in the linkage position of a single fucose subunit (structures shown in FIGS. 26A-26D). The fluorescence chromatogram shows the ability of HILIC chromatography in separating the labeled LNDFH I and LNDFH II isomers (FIG. 25). With the normalized HCD energy kept constant at 28 (arb), one of the major differences in the HCD spectra for the branched isomers is the relative abundance of the fragmentations and the types of fragmentations (FIGS. 26A-26D). For instance, the HCD spectra for the labeled and protonated LNDFH II shows unique fragmentations including the ^{1,5}X_{2α}, Y_{2α}, and Z_{2α} product ions, which are absent for the labeled and protonated LNDFH I. This is particularly due to the branching nature of the glycan which, upon fragmentations taking place at saccharide unit 2 (or saccharide unit 2α for LNDFH II), results in a loss of four saccharide residues for LNDFH I but three saccharide residues for LNDFH II. This can be further exemplified by comparing the base peak pertaining to the Y_{2α} product ion for LNDFH II, which is at a higher mass-to-charge ratio than the base peak pertaining to the Y₂ product ion for LNDFH I Likewise, an example can be drawn from the methylated species where upon normalized HCD energy of 32 (arb), the fragmentations associated with the LNDFH II 2a product ions are also unique. For both the protonated and methylated species, the abundances and types of fragmentations are relatively greater for the LNDFH II isomer relative to the LNDFH I isomer.

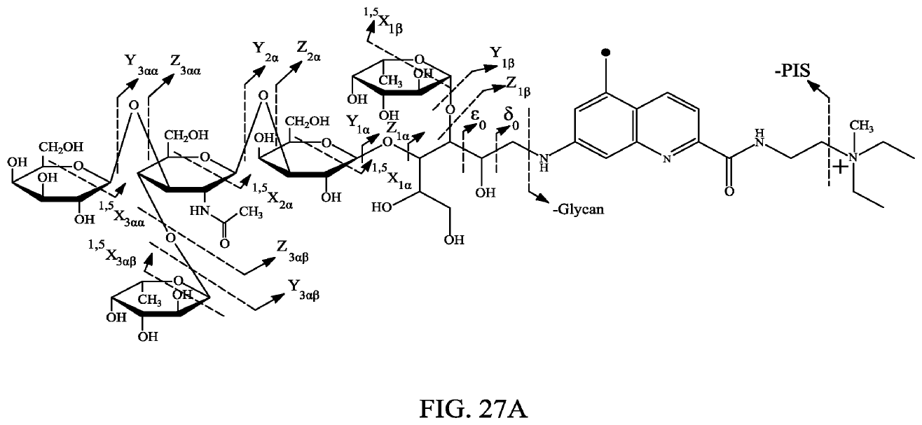

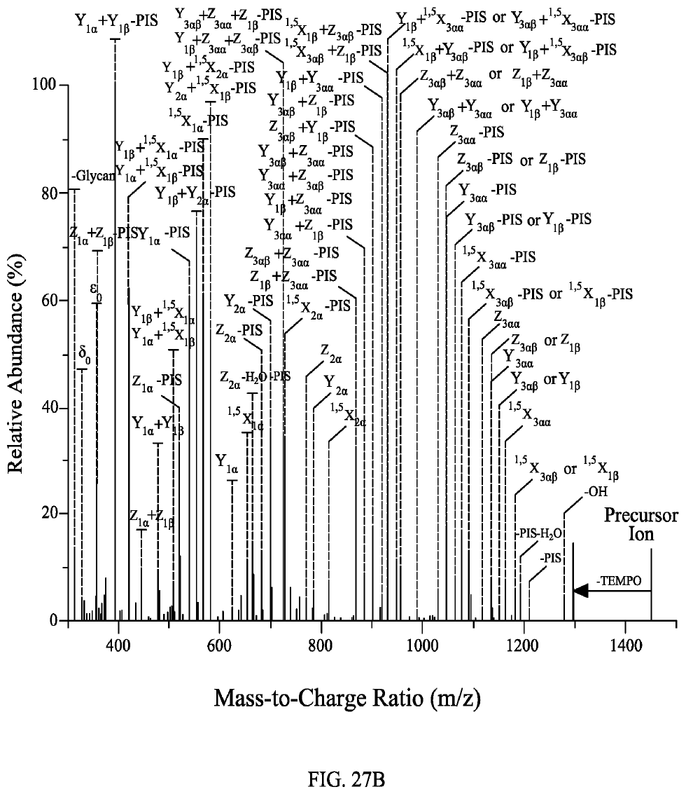

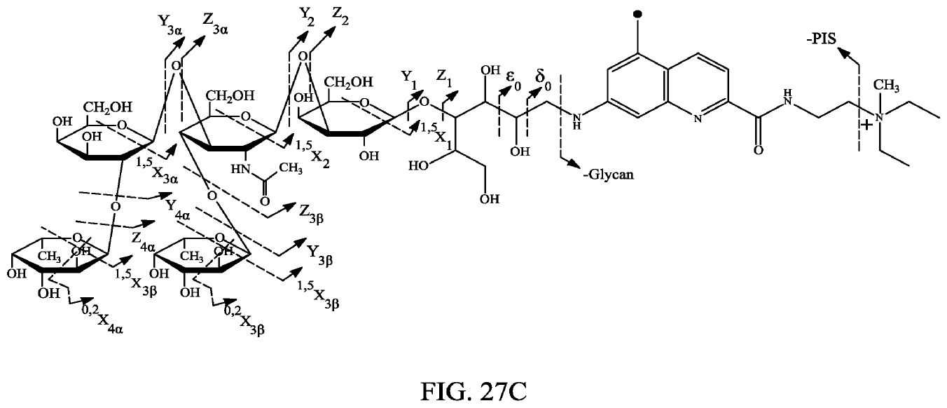

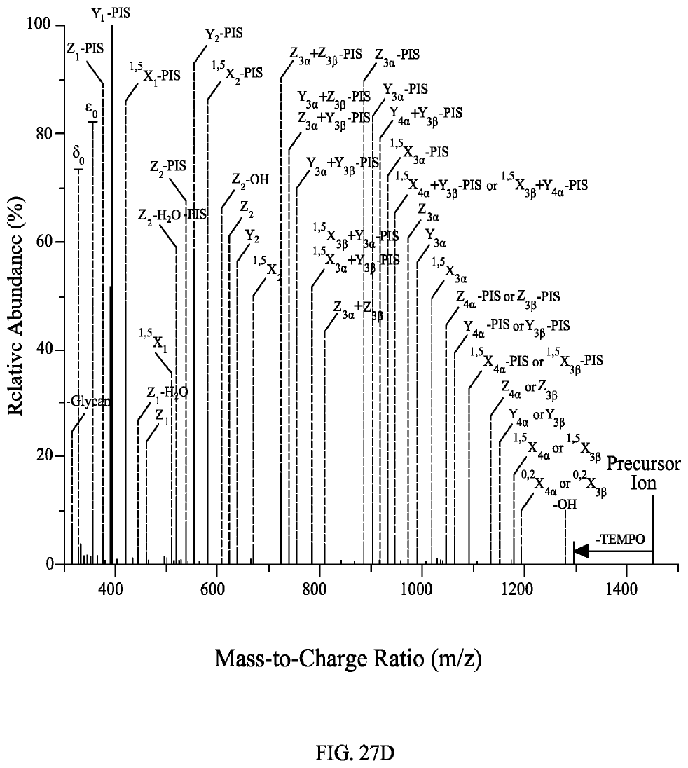

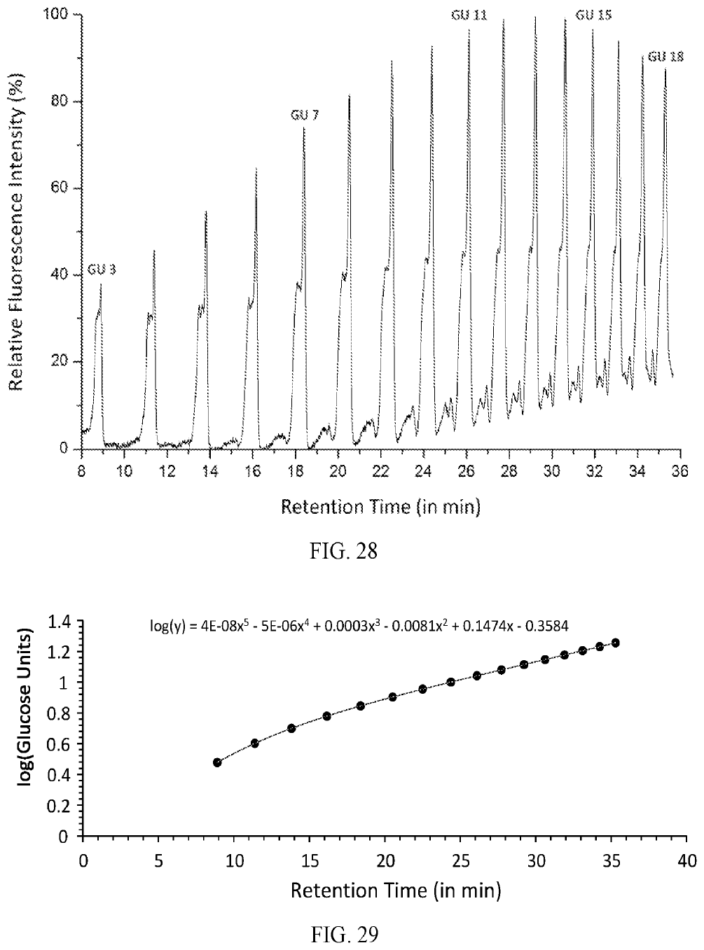

To normalize the retention time of the glycans, a dextran ladder standard solution was prepared and tagged with the novel tagging reagent. A dextran ladder standard is often used in glycan analysis to define the retention time of glycans in terms of glucose units. As shown in FIGS. 27A-27D, the dextran ladder was separated and detected via fluorescence and each peak was defined in terms of the number of glucose units. In a separate experiment not described herein, the mass-to-charge ratio of the analytes at every peak was further validated by mass spectrometry. Based on the data obtained by the tagged dextran ladder, the logarithm of glucose units versus retention time is plotted to construct a curve and obtain an equation (FIG. 28). The equation could then be used to convert the retention time of all glycan analytes obtained from future experiments into glucose units. By describing the glycan analytes in terms of glucose units, the observed retention time from one method can be compared with those acquired by other methods. Because the retention time of the glycan analyte is relative to the retention time of the dextran ladder for a particular method, all glycans can be assigned with a single glucose unit literature value. Ultimately, the glucose unit values, despite the method used to acquire the data, could be utilized as a preliminary glycan characterization technique prior to the more extensive mass spectrometric free radical mediated characterization. Additionally, the characterization of glycans via their observed glucose unit value, coupled with free radical mediated characterization via mass spectrometry, allows for a more intricate and powerful technique of glycan characterization. For instance, by utilizing a HILIC-FLD-MS approach, the theoretical probability of observing a pair of structurally or stereochemically different glycans that have the same glucose unit value, the same exact mass, and the same mass spectrometric fragmentation pattern is extremely low.

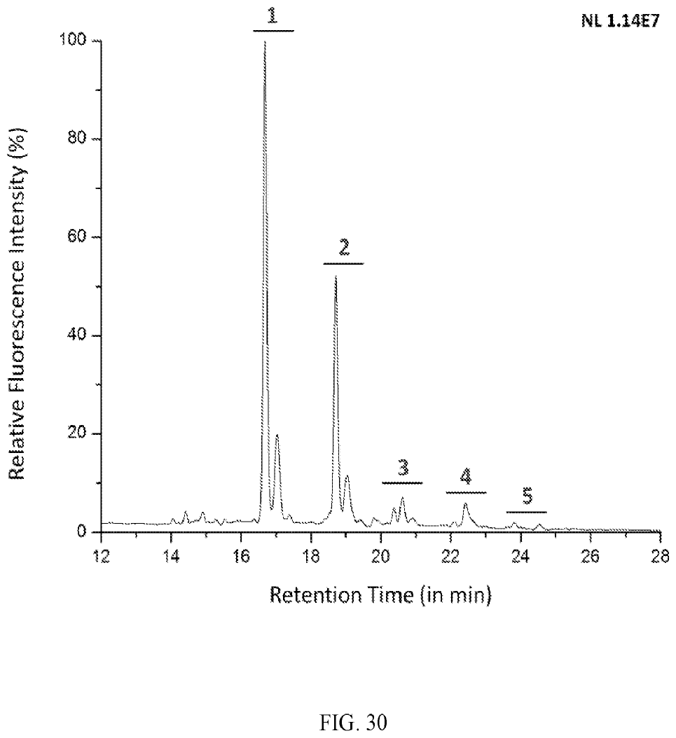

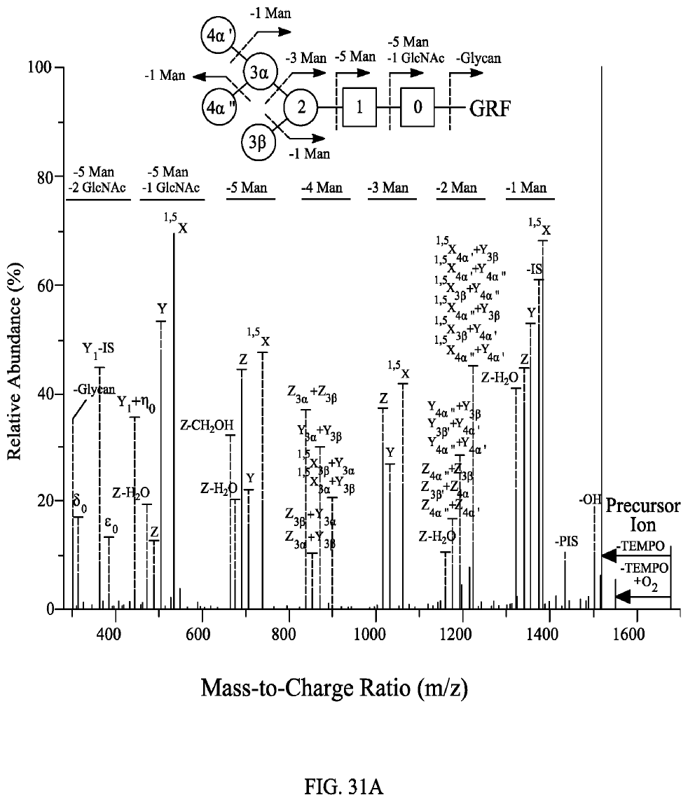

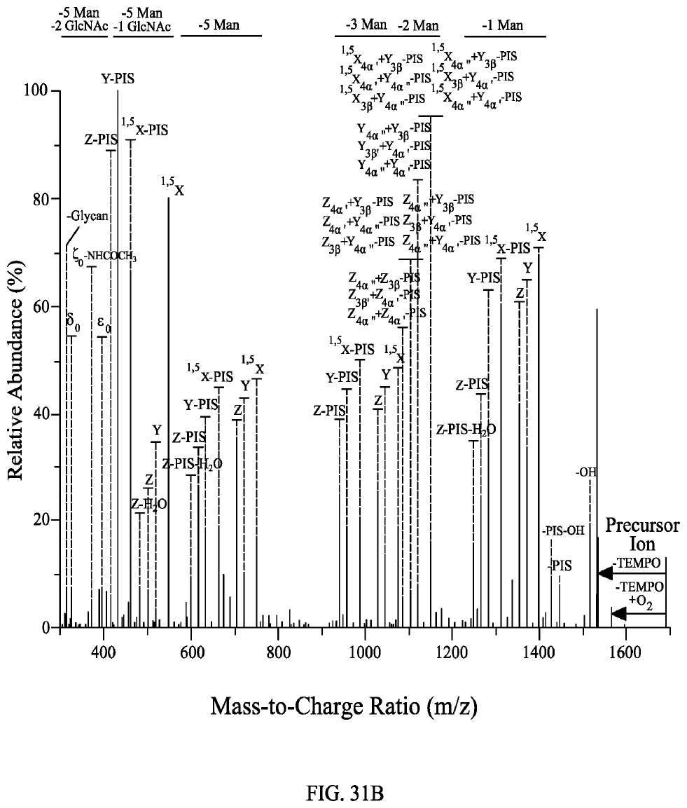

An experiment involving the analysis of N-glycans from ribonuclease B (RNase B) was performed where they were first enzymatically liberated from the asparagine residues by way of peptide-N-glycosidase F (PNGase F). Rnase B is typically employed as a positive control for endoglycosidases and is a high mannose glycoprotein with N-glycans that consist of the structure Man₅₋₉(GlcNAc)₂. After enzymatic liberation, the high mannose N-glycans were tagged with the novel tag through a reductive amination reaction to eliminate the possibility for the interconversion between the reducing end isomers. As portrayed in the fluorescence chromatogram of tagged N-glycans in FIG. 30, Man₅(GlcNAc)₂ had the highest abundance followed by Man₆(GlcNAc)₂. and Man₇₋₉(GlcNAc)₂ had the lowest abundance, which was consistent with the literature data obtained from the glycoprotein manufacturer. Upon a closer inspection of the HCD mass spectrum for Man₅(GlcNAc)₂, the fragmentations provide evidence behind the nature of the isomeric structure. For instance, the presence of the Z_{3α}+Z_{3β} fragmentation under the loss of four mannose subunits can only be acquired with the structure shown in FIG. 31 A rather than the linear isomer counterpart. Additionally, the intensity of the loss of two mannose subunits and four mannose subunits are relatively lower than the loss of one mannose, three mannose, and five mannose subunits, which further provides evidence regarding the structure of the glycan. Due to the branching nature of the structure shown in FIG. 31A, fragmentations of the one mannose, three mannose, and five mannose subunits are simple to induce. Conversely, the loss of two mannose and four mannose subunits are much more challenging and are thereby induced through a mobile proton to yield the loss of multiple external residue losses, which are multiple saccharide fragmentations that are catalyzed via a mobile charge. Furthermore, the higher abundance of the fragmentations corresponding with the loss of two mannose subunits relative to those corresponding with the loss of four mannose subunits can be attributed to the number of possible fragmentation pathways. For instance, since the ^{1,5}X product ion under the loss of two mannose subunits has six total possible fragmentation pathways, the probability of yielding such a fragmentation becomes greater relative to the two total possible fragmentation pathways for the ^{1,5}X product ion associated with the loss of four mannose subunits. As displayed in FIG. 31B, which are fragmentations belonging to the labeled glycan that was methylated to produce a quaternary amine at the ionization site, the only possible pathways for the loss of two mannose subunits (and similarly for the loss of four mannose subunits) is subsequent to the partial fragmentation of the ionization site, which is proposed to yield a mobile proton (Scheme 2). This observation provides additional evidence that supports the isomeric structure and branching locations of the mannose subunits for Man₅(GlcNAc)₂ as shown in FIG. 31A.

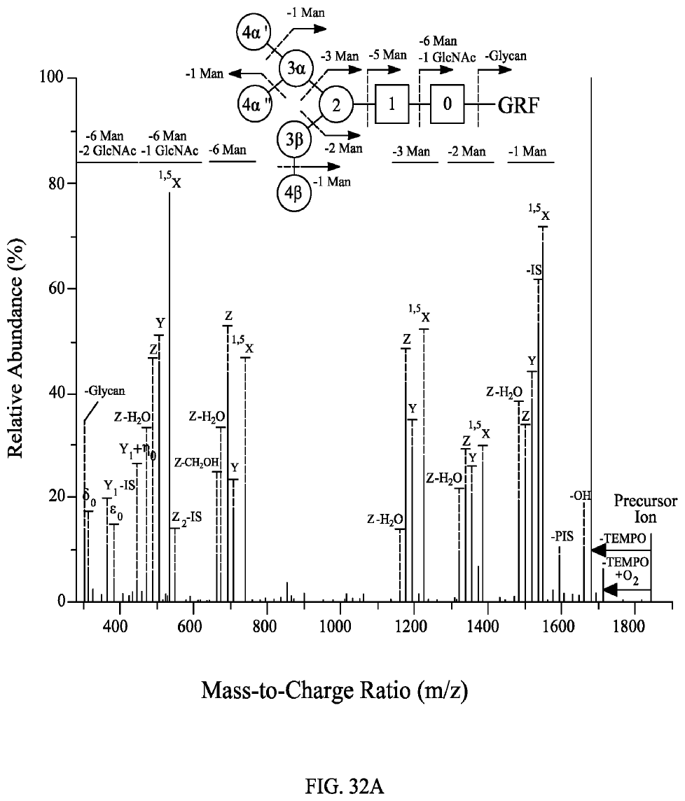

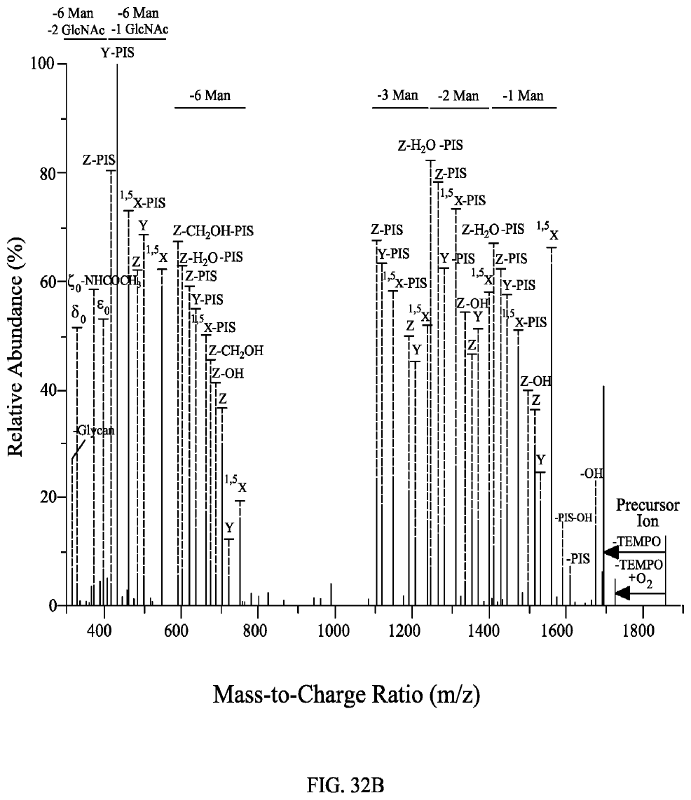

Likewise, the HCD mass spectra for Man₆(GlcNAc)₂ provides evidence that the predominant isomer present on RNase B is biantennary with a single saccharide elongation taking place at the 3β unit from the Man₅(GlcNAc)₂ predecessor (FIG. 32A). In its protonated and labeled form (FIG. 32A), HCD of Man₆(GlcNAc)₂ results in highly-abundant fragmentations that are associated with the loss of one, two, three, and six mannose residues, but not four and five mannose residues. Similarly, the methylated and labeled Man₆(GlcNAc)₂ yields similar product ions as the protonated with the addition of the fragmentations occurring alongside the partial loss of the ionization site (FIG. 32B). The proposed isomeric structure of Man₆(GlcNAc)₂ therefore satisfies the fragmentation data, and vice versa.

### Conclusion

For optimal LC-MS analysis coupled with fluorescence detection and free radical-mediated characterization of glycans, the derivatization of such biomolecules must be conducted with a tagging reagent that is first carefully defined by the incorporation of multiple important chemical sites. Among these sites, as mentioned previously, include the fluorophore base, an ionization and methylation site with a high affinity for positively charged ions, a glycan labeling site to form a stable covalent attachment, and the free radical precursor which facilitates the generation of systematic fragmentations, which all commercially available tags collectively lack. The fluorophore base exhibits the fluorescence capabilities required for detection and quantitation abilities of glycans in a quantity-dependent manner. The purpose of utilizing fluorescence is to maximize the sensitivity of liquid chromatographic detection and quantitation methods. The basic site allows for the feasible induction of a positive charge via either protonation, ion-dipole interactions, or methylation. The importance behind the recruitment of the basic site is to allow for ionization to take place during or prior to mass spectrometric analysis of glycans, thereby significantly enhancing the mass spectrometric signal-to-noise ratio. The glycan labeling site is the most essential feature to include on the tag as it selectively reacts with the unique glycan reducing terminus. Lastly, the free radical precursor allows for the low-energy formation of a nascent free radical by dissociation techniques such as higher-energy collision dissociation (HCD) or collision-induced dissociation (CID). Ultimately, the free radical simultaneously reacts with the glycan to yield glycan fragmentations that are reproducible, systematic, predictable, diagnostic, and efficient towards glycan characterization, identification, and differentiation among glycans and their isomers. Altogether, the rational recruitment of these chemical sites has constructed a novel tag that is optimal for not only quantitating glycans at low concentrations, which is typical of samples acquired from glycoproteins, but also for pairing the sensitive MS detection with the free radical-mediated glycan characterization capability. Moreover, this novel tag is designed to be chemically stable for significantly longer periods of storage time than reagents that recruit an NHS-carbamate coupling site.

Multiple glycan samples with varying attributes were labeled and analyzed via hydrophilic liquid chromatography-fluorescence detection-mass spectrometry (HILIC-FLD-MS) to fully assess the capabilities of the novel reagent. The maltosaccharides, which consisted of maltose, maltotriose, maltotetraose, maltopentaose, maltohexaose, and maltoheptaose, were premixed in an equimolar mixture prior to labeling and fluorescence quantitation. Next, the longest linear maltosaccharide was subjected to higher-energy collision dissociation to yield information-rich fragmentations via free radical-mediated pathways where both cross-ring fragmentations and glycosidic cleavages were observed throughout the linear biopolymer. To further assess the ability of the novel reagent in yielding glycan fragmentations that allow for discerning the structures of branched isomers, lacto-N-difucohexaose I and lacto-N-difucohexaose II were premixed, labeled, and were subjected to higher-energy collision dissociation. After the separation of the labeled branched isomers via HILIC, the fragmentations of both isomers revealed differences in the abundances and types of fragmentations which reflected their structures. Ultimately, the novel reagent was successful in distinguishing between two branched isomers based solely on their resultant product ions. The novel reagent was further assessed by labeling N-glycans that were first enzymatically liberated from ribonuclease B. The labeled N-glycans were separated via HILIC, detected via fluorescence, and accurately characterized via free radical-mediated characterization. With the use of HILIC for glycan separation paired with fluorescence detection and tandem mass spectrometry for the induction of fragmentations, the novel reagent ultimately provides powerful and modern analytical capabilities. The same analytical capabilities are contemplated for other reagents described herein. As such, the evolvement of this novel analytical technology is expected to allow for further growth within glycan analysis with the potential for the study of glycans as human disease biomarkers and improved quality assurance assays for biopharmaceuticals.

It is to be understood that the invention is not limited to the particular embodiments of the invention described above, as variations of the particular embodiments may be made and still fall within the scope of the appended claims.

The invention will be further described, without limitation, by the following numbered paragraphs:
1) A compound of formula (Ia) or (Ib)

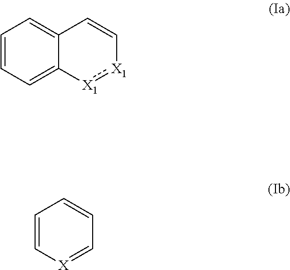

substituted with R¹ and A, optionally with R²,
wherein
[symbol] is a single or a double bond;
if [symbol] is a double bond, then:
   X¹ is C and X² is C or N, or
   X² is C and X¹ is N;
if [symbol] is a single bond, then:
   X₁ is NH, N-alkyl, O, or NO and X² is C═O, or
   X¹ is NH, N-alkyl, or NO and X² is O, or
   X¹ is C═O and X² is NH, N-alkyl, O, or NO, or
   X¹ is O and X² is NH, N-alkyl, or NO;
   X is CH, N or CR⁹;
R¹ is selected from the group consisting of:

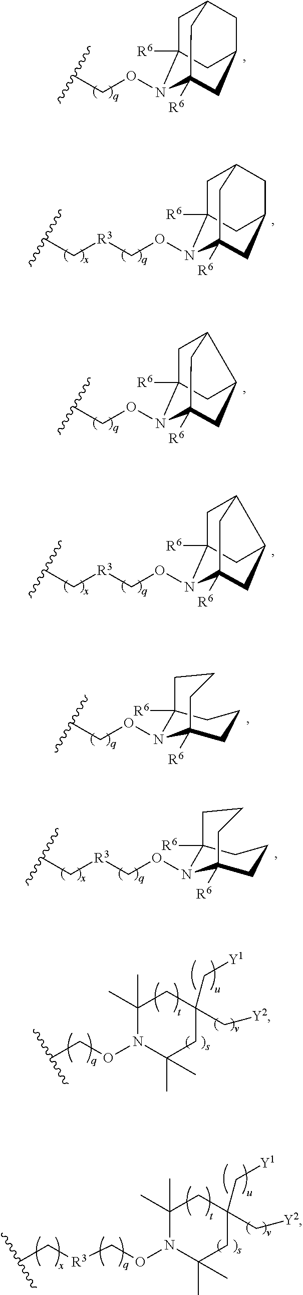

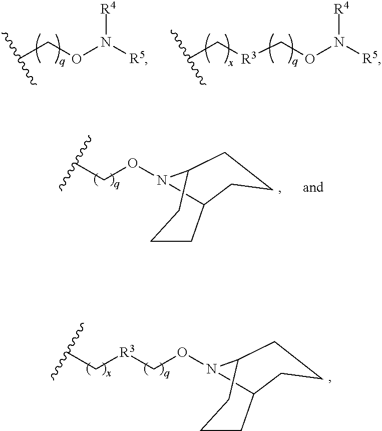

or a derivative thereof,
Y¹ and Y² are each independently selected from the group consisting of:
H, O, OH, CH₃, N₃, CN, NH₂, a carbonyl, R³—(CH₂)_{y}CO(CH₂)ₚCH₃, R³—(CH₂)_{y}COH, R³—(CH₂)_{y}—NH₂, N[(CH₂)_{y}(CH₃)]₂, R³—(CH₂)_{y}—N[(CH₂)ₚCH₃]₂, COOH, R³—(CH₂)_{y}—COOH, R³—(CH₂)_{y}—COO(CH₂)ₚCH₃, R³—(CH₂)_{y}—CN, R³—(CH₂)_{y}—N₃,

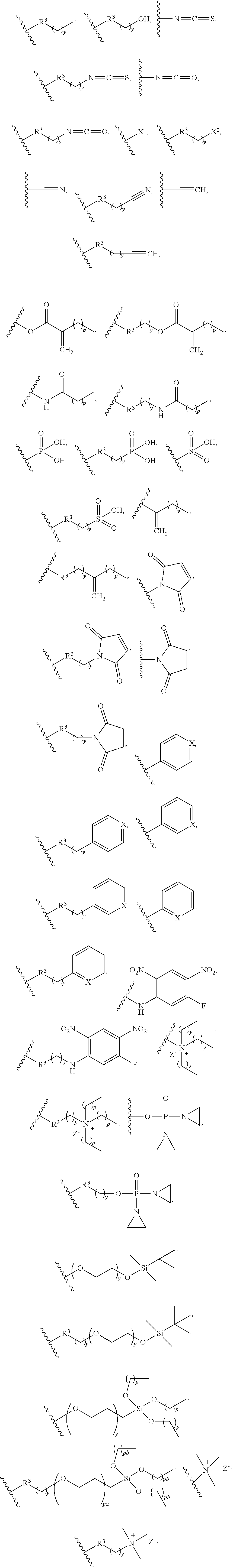

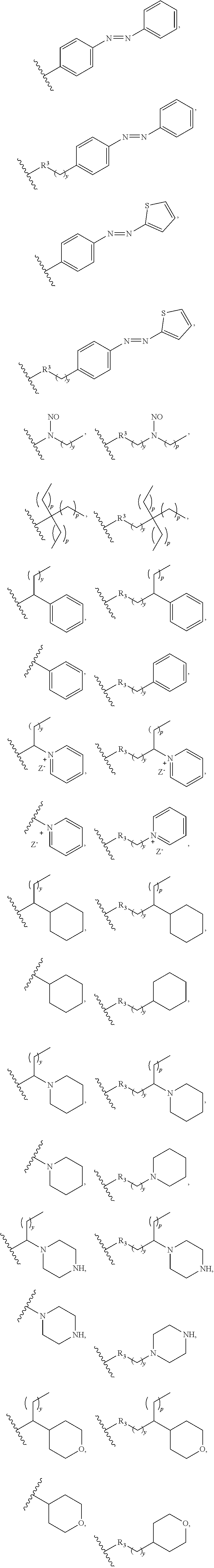

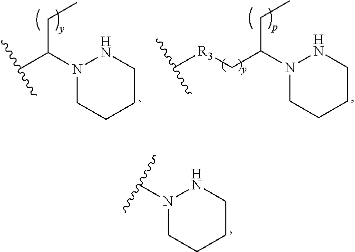

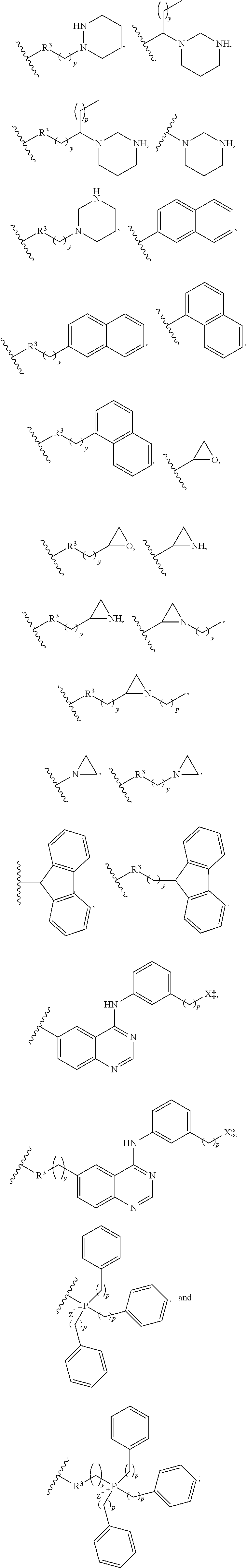

with the proviso that if Y¹ or Y² is a carbonyl, then the other is not a carbonyl,
or, u and v are 0, and Y¹ and Y² form a structure together selected from the group consisting of:

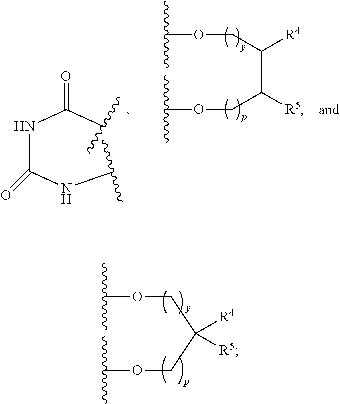

or, Y¹ and Y² function as a linker to a group independently selected from the groups defined in
R¹ wherein the linker is selected from the group consisting of:

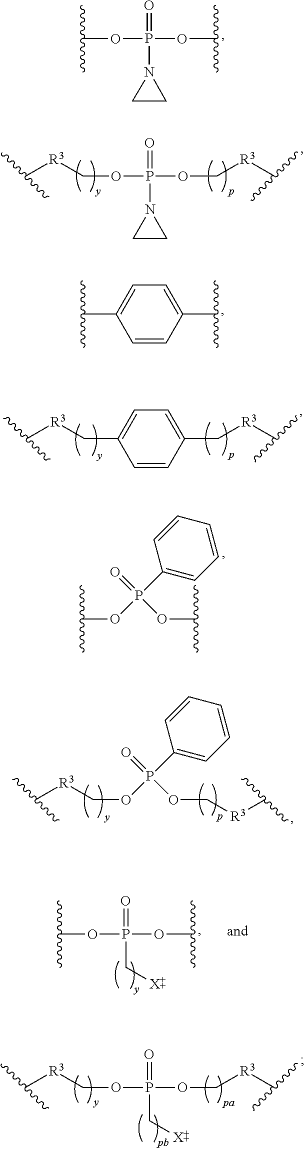

X^{‡} is a halogen;
Z is a counter ion;
R² is selected from the group consisting of

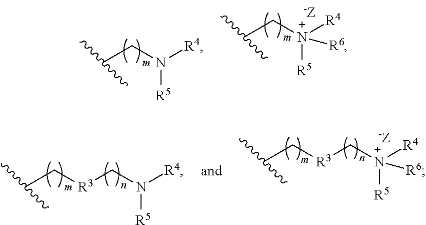

A is selected from the group consisting of

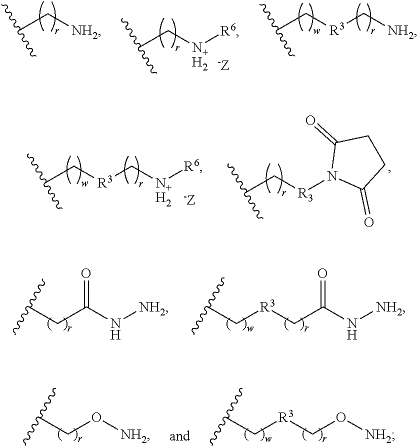

R³ are each independently selected from the group consisting of: substituted nitrogen, oxygen, carbonyl, amide, ester, ether, urea, hydrazide, carbamate, carbonate, thiocarbonate, thiol, thiourea, sulfur, sulfoxide, and sulfone;
R⁴, R⁵, and R⁶ are each independently selected from the group consisting of: H, O, OH, carbonyl, CN, N₃, COOH, alkyl, t-butyl, sec-butyl, isobutyl, isopropyl, tetrahydropyran, alkyl amino, alkylsulfonic acid, alkyl phosphonic acid, R³—(CH₂)_{y}CO(CH₂)ₚCH₃, R³—(CH₂)_{y}COH, NH₂, R³—(CH₂)_{y}—NH₂, N[(CH₂)_{y}(CH₃)]₂, R³—(CH₂)_{y}—N[(CH₂)ₚCH₃]2, R³—(CH₂)_{y}—COOH, R³—(CH₂)_{y}—COO(CH₂)ₚCH₃, R³—(CH₂)_{y}— R³—(CH₂)_{y},—N₃, CH₃,

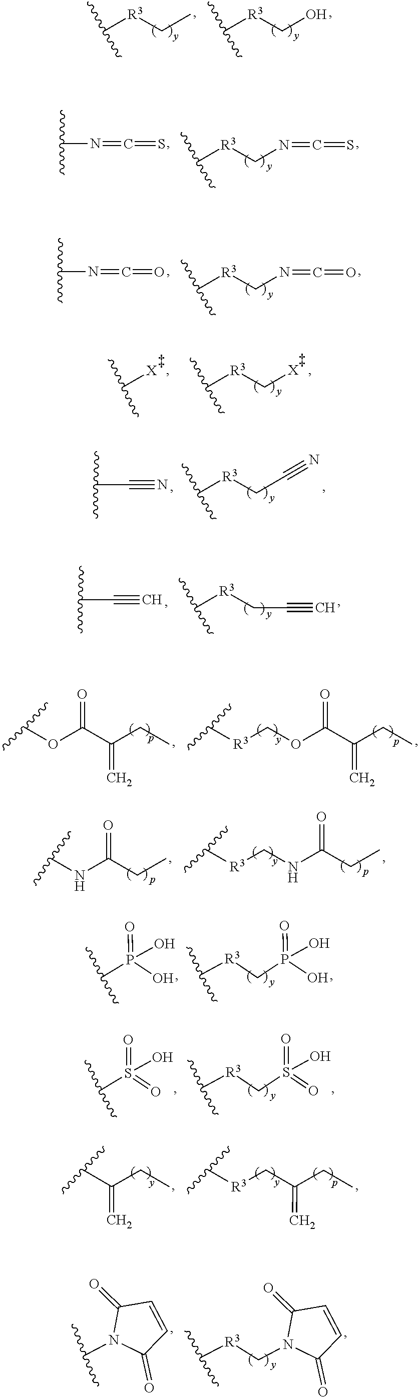

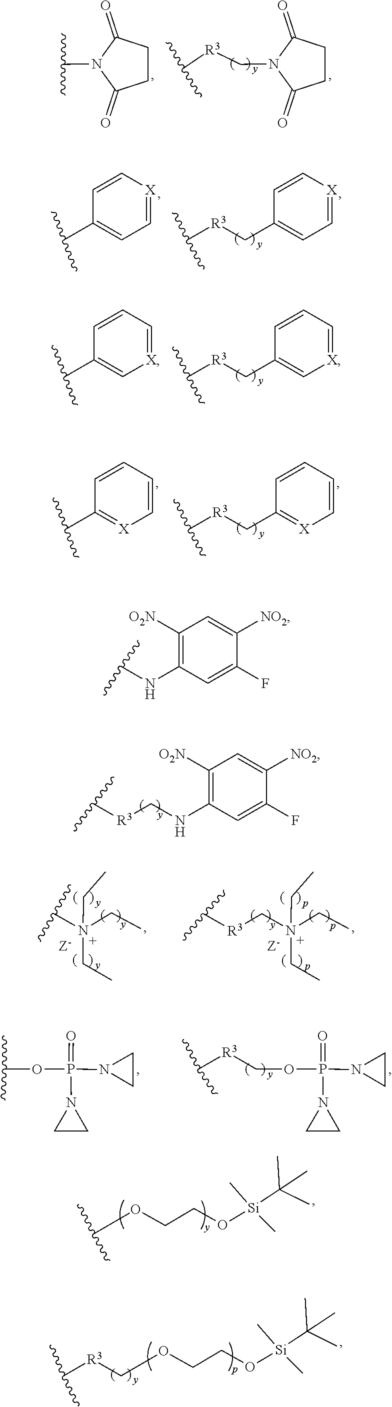

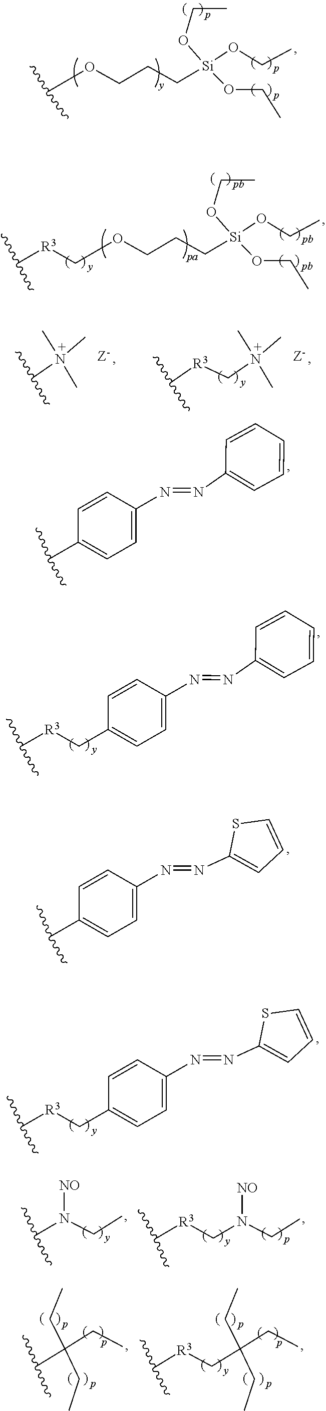

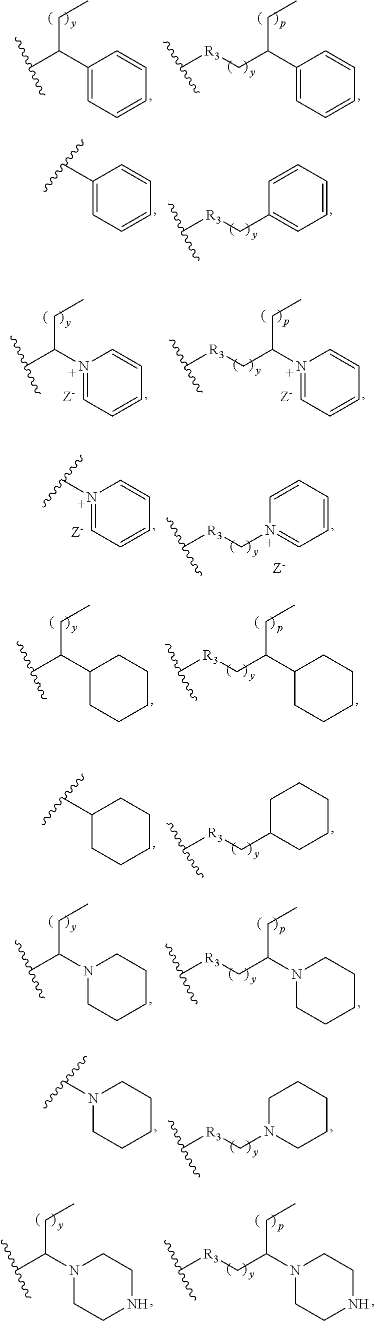

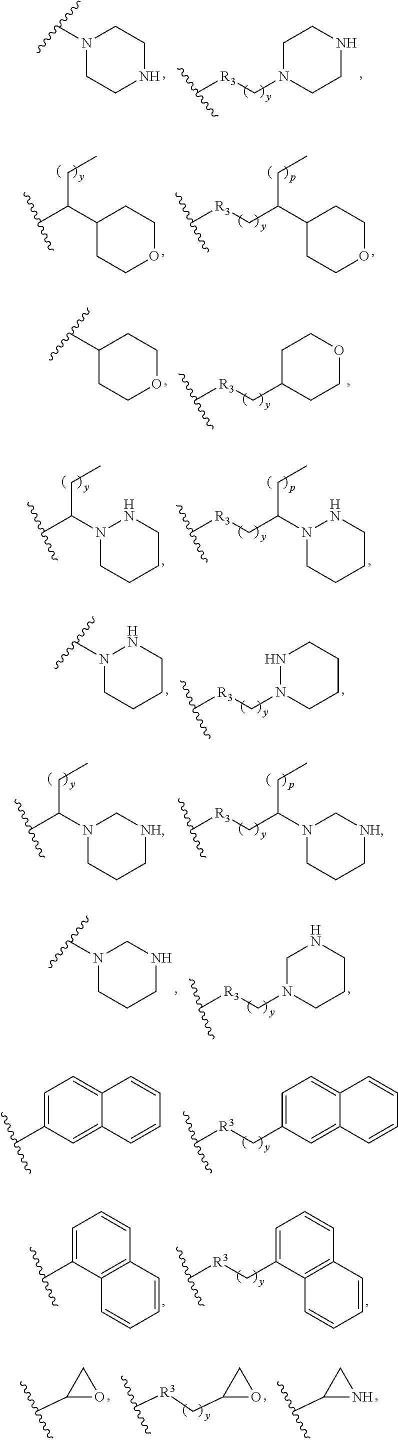

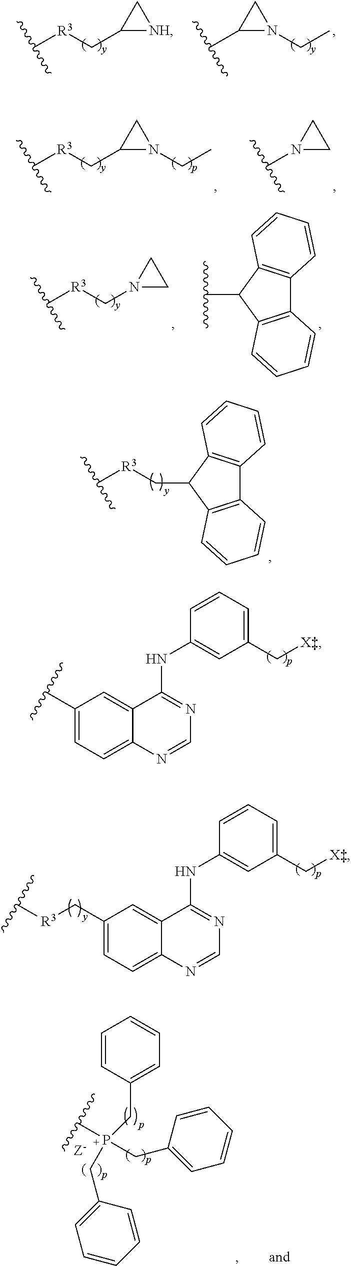

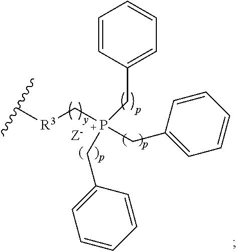

or R⁴ and R⁵ together with the nitrogen to which they are attached may form an optionally substituted 5- to 8-membered saturated or partially unsaturated ring;
or R⁴ and R⁵ function as a linker to a group independently selected from a group defined in R¹; wherein the linker is selected from the group considering of:

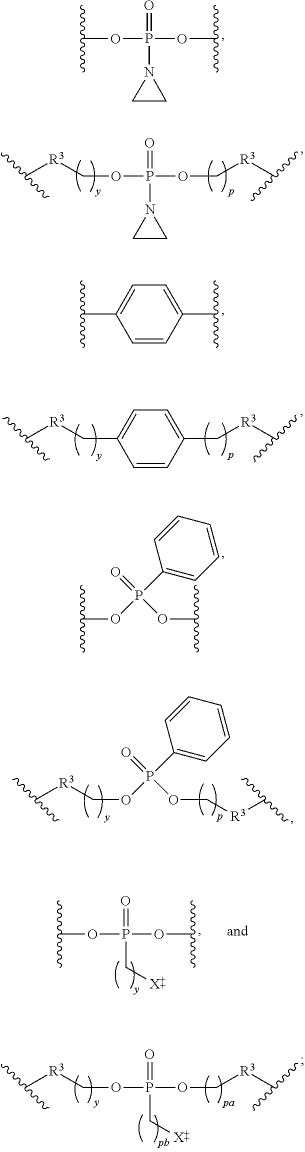

x, q, t, v, s, u, m, n, r, w, y, p, pₐ, and p_{b} are each independently an integer from 0 to 24;
R⁷ is a secondary, tertiary, reduced amine, an ether, or an alcohol;
R⁸ is an alkylated or hydrogenated imide, or an oxygen;
R⁹ is

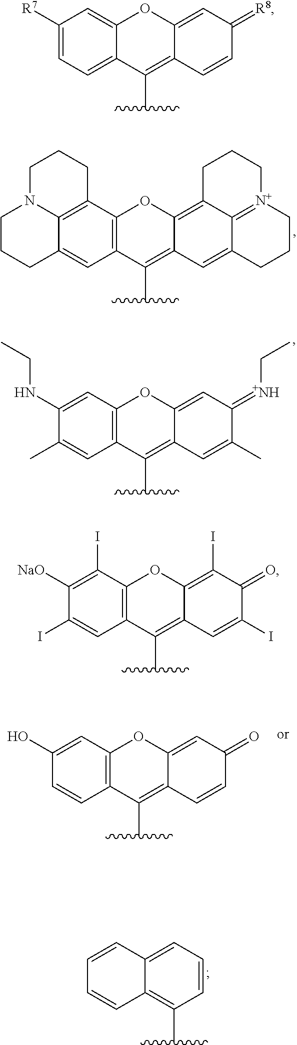

wherein any hydrogen atom may be substituted with a fluorine, deuterium, or tritium atom;
with the proviso that if the structure is a compound of formula (Ib), then formula (Ib) is substituted with R¹, R² and A, with the proviso that if X₁ is N and X₂ is C, and R₁ is

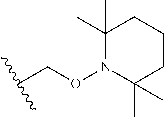 [SMILES: *CON1C(C)(C)CCCC1(C)C]

and one of R² or A is H, then R² or A is not

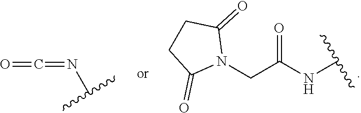

2) The compound of paragraph 1, which is substituted with R².
3) The compound of paragraph 1 having formula (Ic)

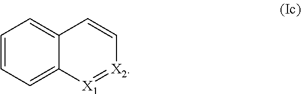

4) The compound of paragraph 1 having formula (Id), formula (Ie), or formula (If)

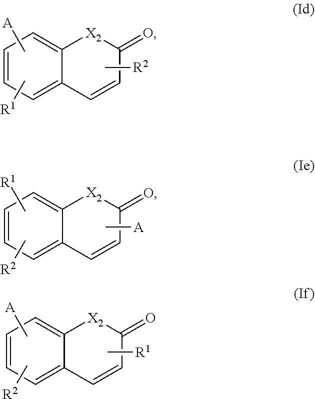

wherein X² is NH, N-alkyl, or NO.
5) The compound of paragraph 1 which is

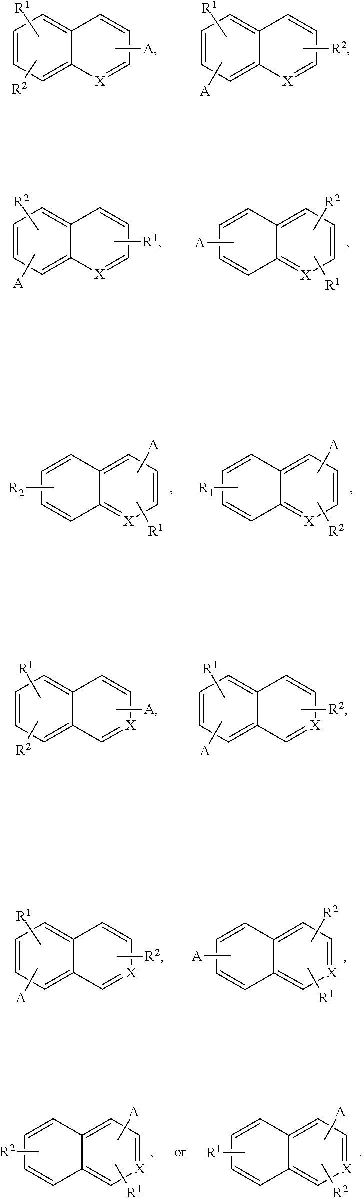

6) The compound of paragraph 1 which is

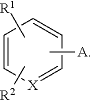

7) The compound of paragraph 1 which is

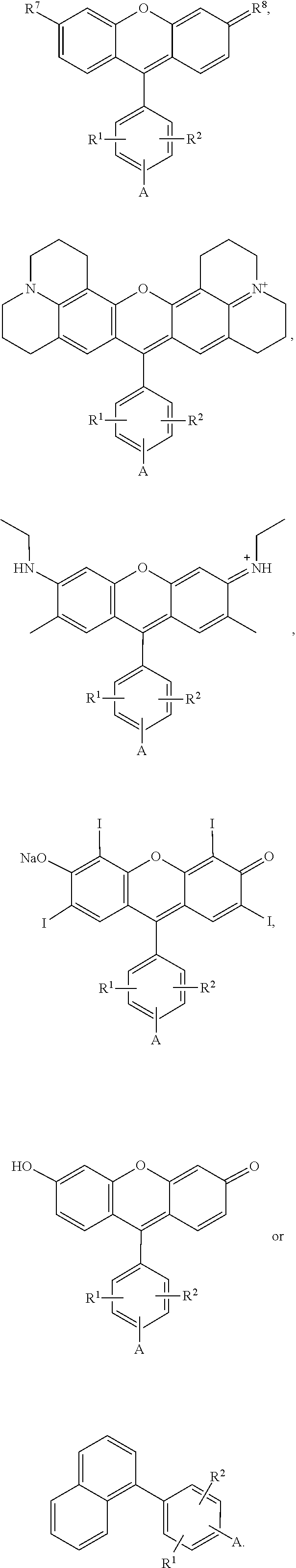

8) A compound which is

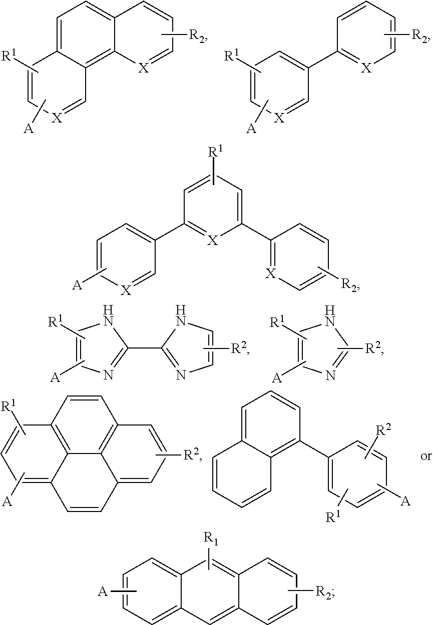

wherein X, A, R₁ and R² are defined as recited in paragraph 1.
9) The compound of paragraph 1, wherein R₁ is

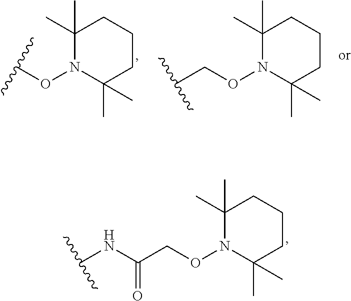

wherein R₁ is selected from one of the following structures

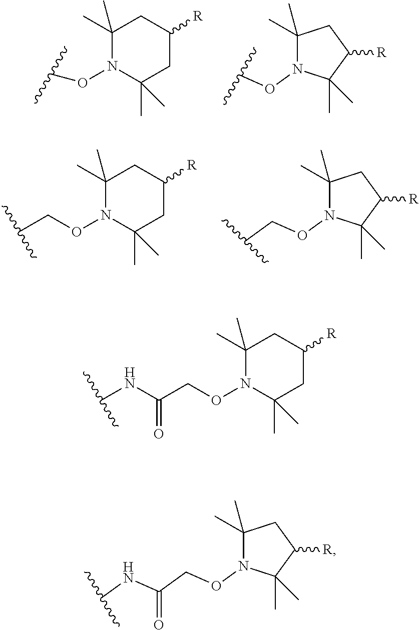

wherein R is independently selected from H, alkyl, alkyl amino, ester, ether, OH, ═O, COOH, NH₂, alkyl phosphonic acid, and halide.
10) The compound of paragraph 1, wherein R₂ is

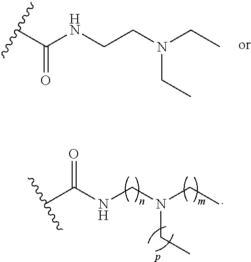

11) The compound of paragraph 1, wherein A is NH₂,

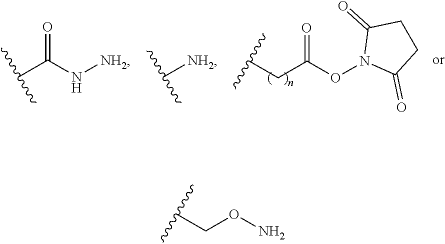

12) The compound of paragraph 5 wherein X is CH, or N.
13) The compound of paragraph 1, which is:

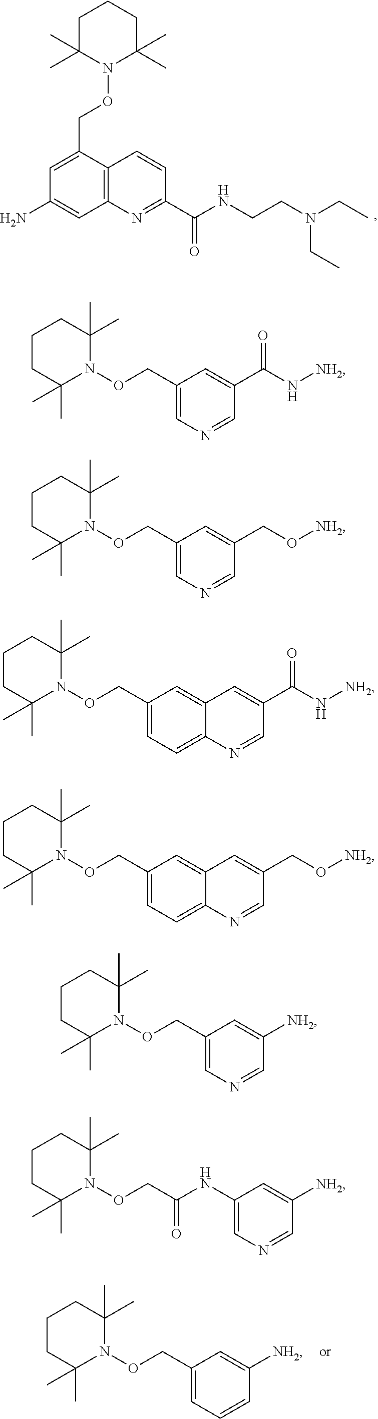

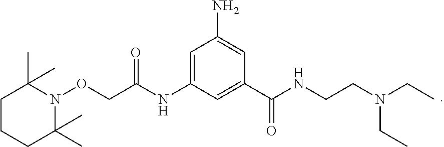 [SMILES: CCN(CC)CCNC(=O)c1cc(N)cc(NC(=O)CON2C(C)(C)CCCC2(C)C)c1]

14) A method of quantitating and/or characterizing a glycan comprising:
   a) obtaining the compound of paragraph 1;
   b) contacting the compound with the glycan, thereby forming a labeled glycan.
15) The method of paragraph 14, further comprising dissociation of the labeled glycan to form glycan fragmentations.
16) The method of paragraph 15, wherein the dissociation is collisional-induced dissociation (CID) or higher-energy collision dissociation (HCD).
17) The method of paragraph 14, further comprising analyzing the labeled glycan or glycan fragmentations with an instrument which is a fluorimeter, a mass spectrometer or liquid chromatography instrument, optionally with an instrument capable of detecting fluorescence and/or absorbance.
18) The method of paragraph 14, wherein the glycan comprises a reducing terminus.
19) The method of paragraph 14, wherein the glycan is other than an N-glycan.
20) The method of paragraph 14 wherein the instrument is a Ultra-Performance Liquid Chromatography (UPLC), a linear quadrupole ion trap (LTQ-XL) mass spectrometer, a Q Exactive Orbitrap mass spectrometer, or a liquid chromatography-mass spectrometry (LC-MS).
21) The method of paragraph 14 wherein the instrument is equipped with an electrospray ionization (ESI), heated-electrospray ionization (HESI) source and/or with a fluorescence detector.
22) The compound of paragraph 1 for use in the preparation of a labeled glycan or glycan fragmentations, or for use in glycan detection or glycan quantitation.

It is to be understood that the invention is not limited to the particular embodiments of the invention described above, as variations of the particular embodiments may be made and still fall within the scope of the appended claims.

What is claimed is:

1. A compound of formula (Ia) or (Ib) 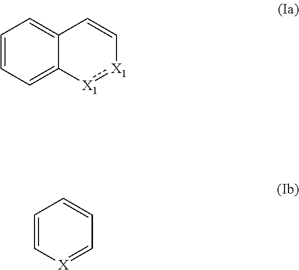
substituted with R¹ and A, optionally substituted with R²,
wherein [symbol]
is a single or a double bond;
if [symbol] is a double bond, then:
X¹ is C and X² is C or N, or
X² is C and X¹ is N;
if [symbol] is a single bond, then:
X¹ is NH, N-alkyl, O, or NO and X² is C═O, or
X¹ is NH, N-alkyl, or NO and X² is O, or
X¹ is C═O and X² is NH, N-alkyl, O, or NO, or
X¹ is O and X² is NH, N-alkyl, or NO;
X is CH, N or CR⁹;
R¹ is selected from the group consisting of 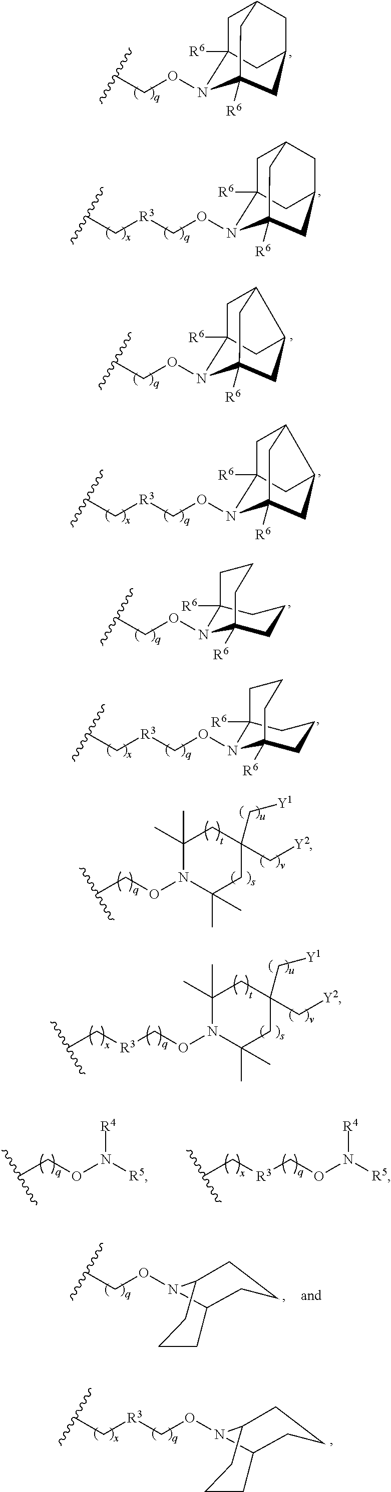
or a derivative thereof,
Y¹ and Y² are each independently selected from the group consisting of:
H, O, OH, CH₃, N₃, CN, NH₂, R³—(CH₂)_{y}CO(CH₂)ₚCH₃, R³—(CH₂)_{y}COH, R³—(CH₂)_{y}—NH₂, N[(CH₂)_{y}(CH₃)]₂, R³—(CH₂)_{y}—N[(CH₂)ₚCH₃]₂, COOH, R³—(CH₂)_{y}—COOH, R³—(CH₂)_{y}—COO(CH₂)ₚCH₃, R³—(CH₂)_{y}—CN, R³—(CH₂)_{y}—N₃, 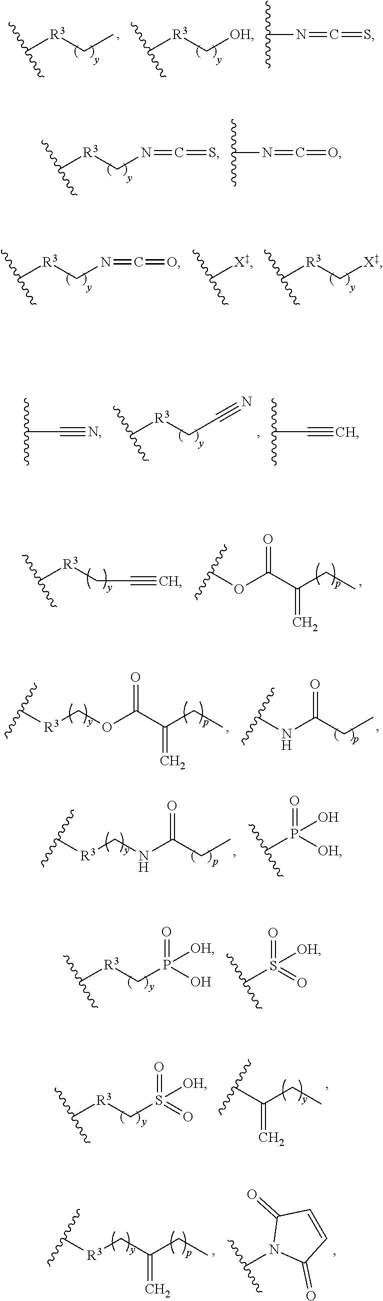 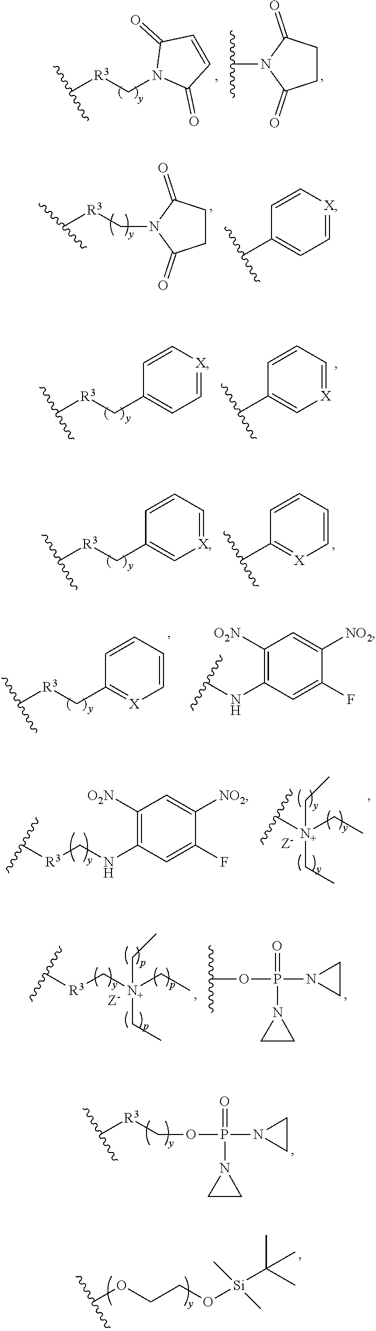 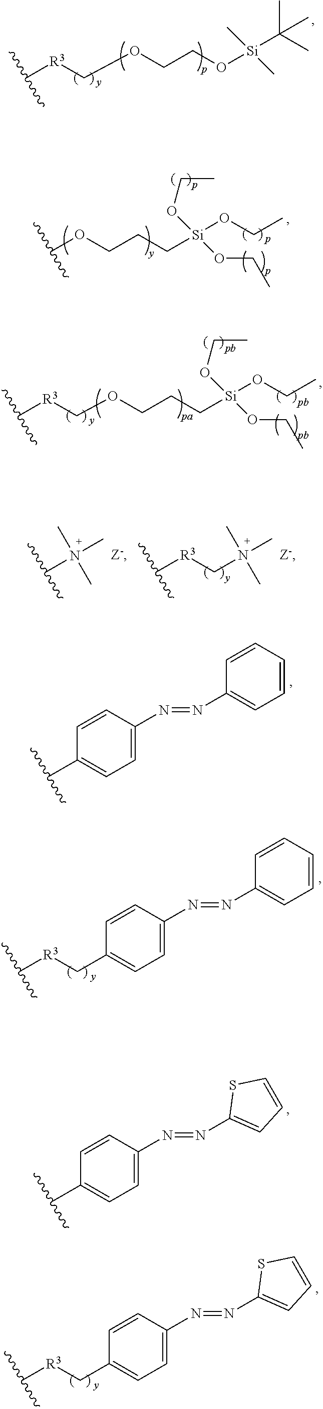 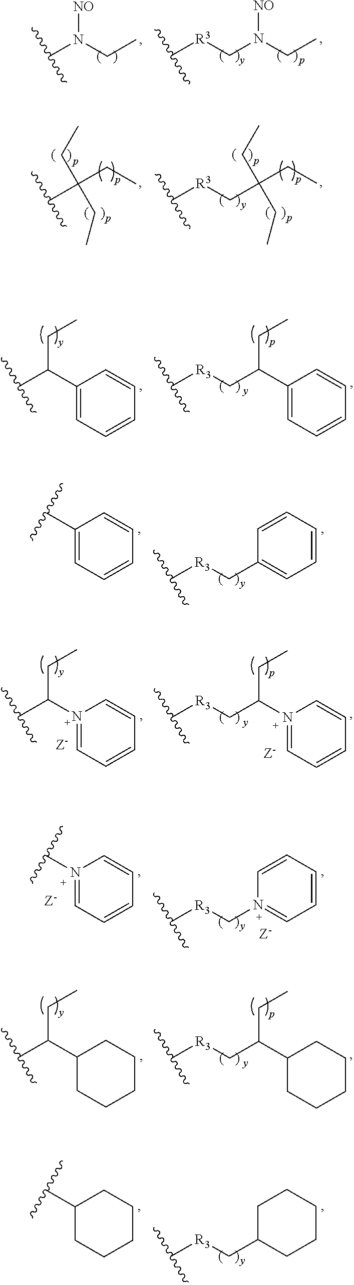 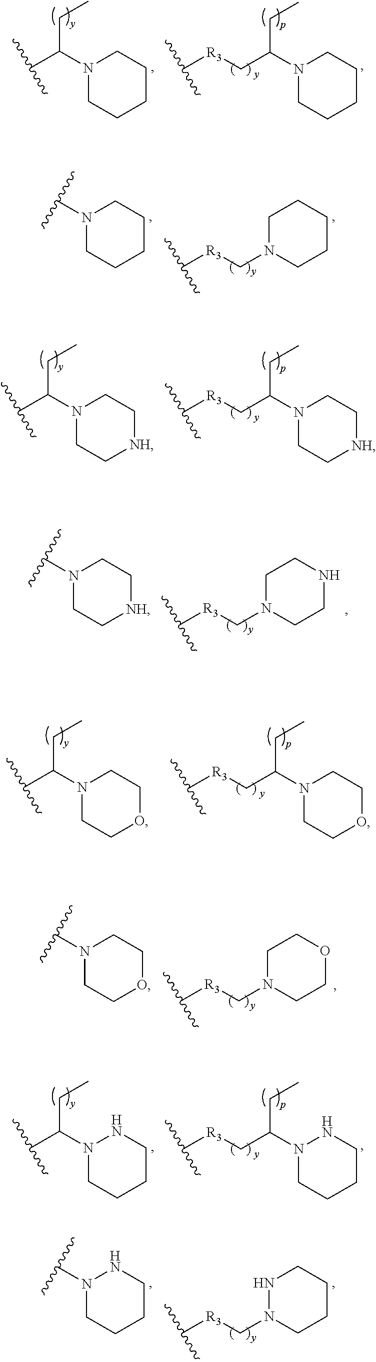 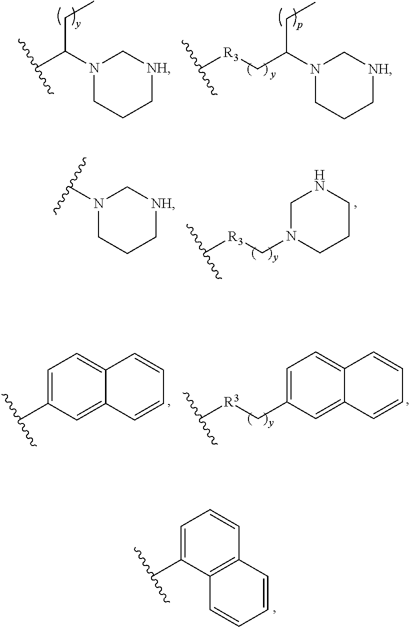 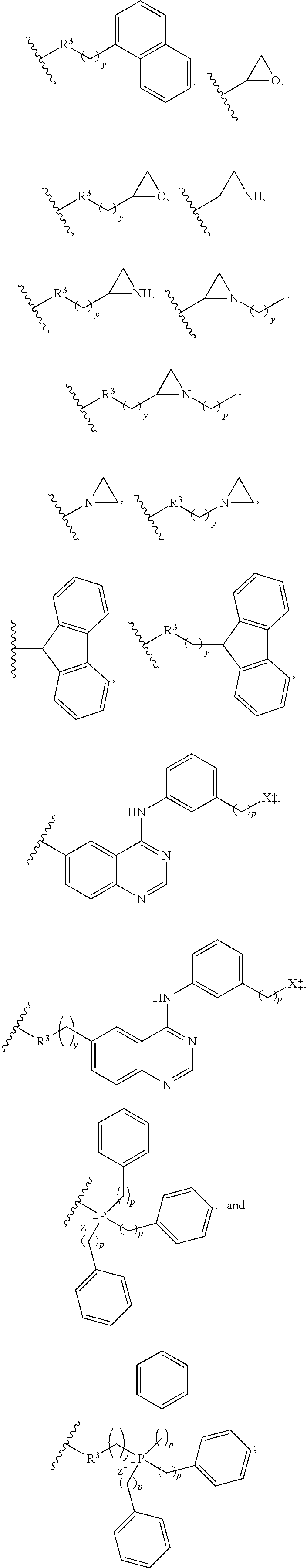
with the proviso that if Y¹ or Y² is a carbonyl, then the other is not a carbonyl,
or, u and v are 0, and Y¹ and Y² form a structure together selected from the group consisting of: 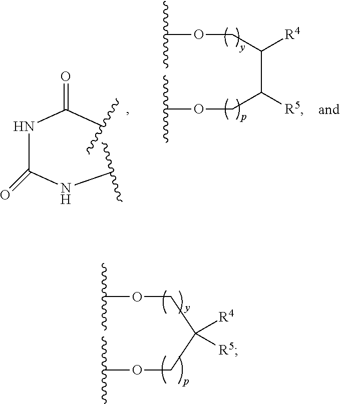
or, Y¹ and Y² function as a linker to a group independently selected from the groups defined in R¹ wherein the linker is selected from the group consisting of: 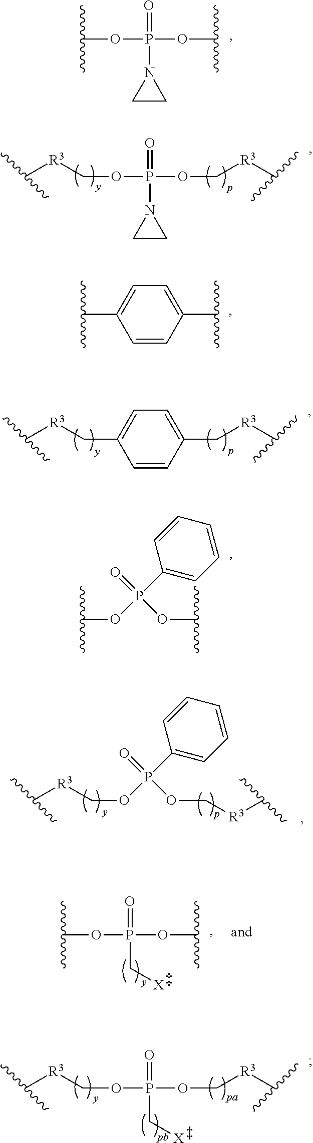
X^{‡} is a halogen;
Z is a counter ion;
R² is selected from the group consisting of 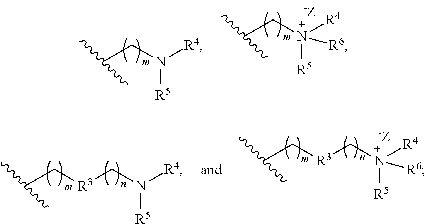
A is selected from the group consisting of 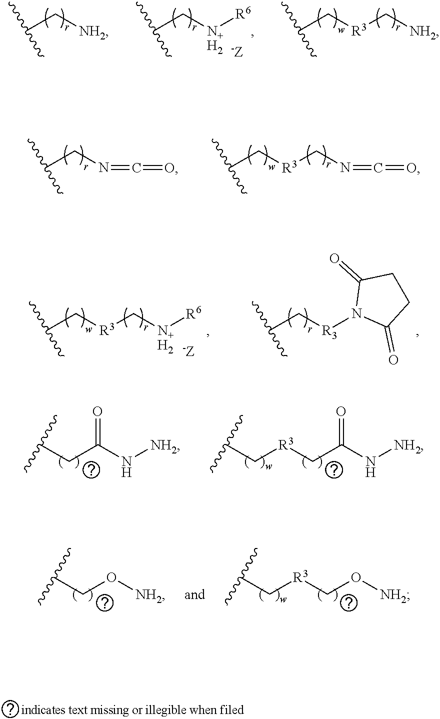
R³ are each independently selected from the group consisting of: substituted nitrogen, oxygen, carbonyl, amide, ester, ether, urea, hydrazide, carbamate, carbonate, thiocarbonate, thiol, thiourea, sulfur, sulfoxide, and sulfone;
R⁴, R⁵, and R⁶ are each independently selected from the group consisting of: H, OH, O, CN, N₃, COOH, alkyl, t-butyl, sec-butyl, isobutyl, isopropyl, tetrahydropyran, alkyl amino, alkylsulfonic acid, alkyl phosphonic acid, R³—(CH₂)_{y}CO(CH₂)ₚCH₃, R³—(CH₂)_{y}COH, NH₂, R³—(CH₂)_{y}—NH₂, N[(CH₂)_{y}(CH₃)]₂, R³—(CH₂)_{y}—N[(CH₂)ₚCH₃]₂, R³—(CH₂)_{y}—COOH, R³—(CH₂)_{y}—COO(CH₂)ₚCH₃, R³—(CH₂)_{y}— R³—(CH₂)_{y}—N₃, CH₃, 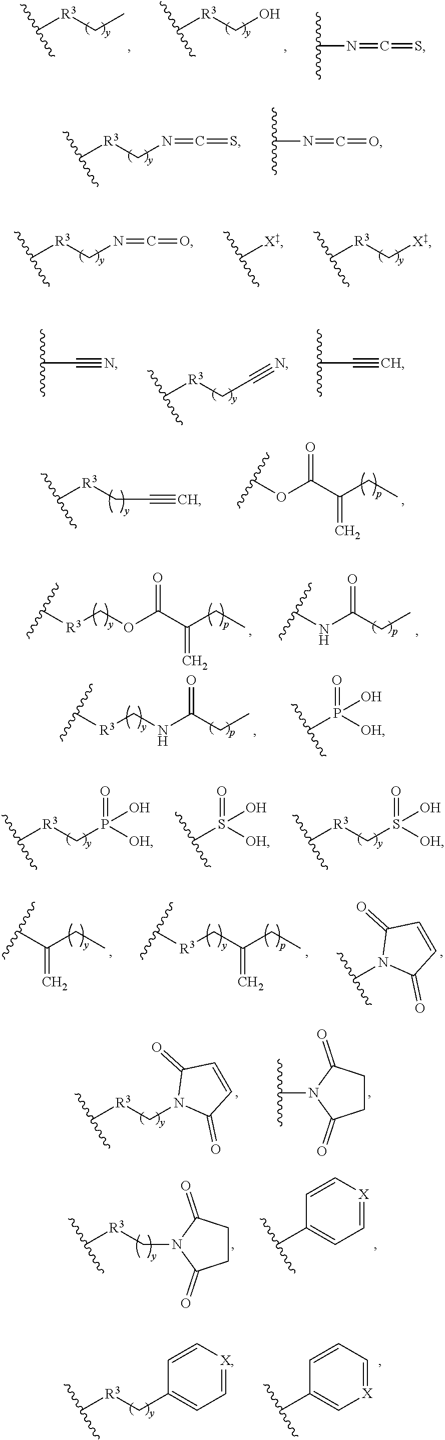 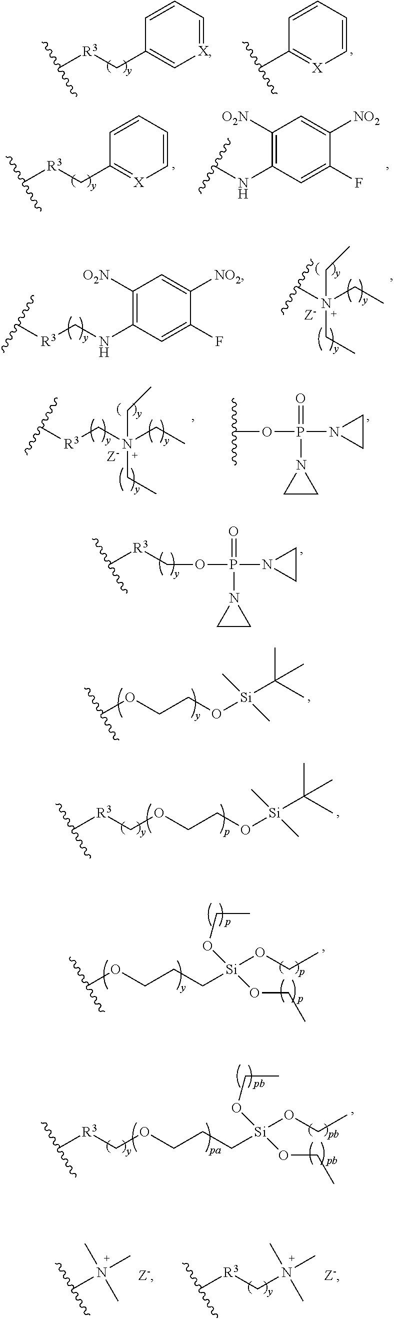 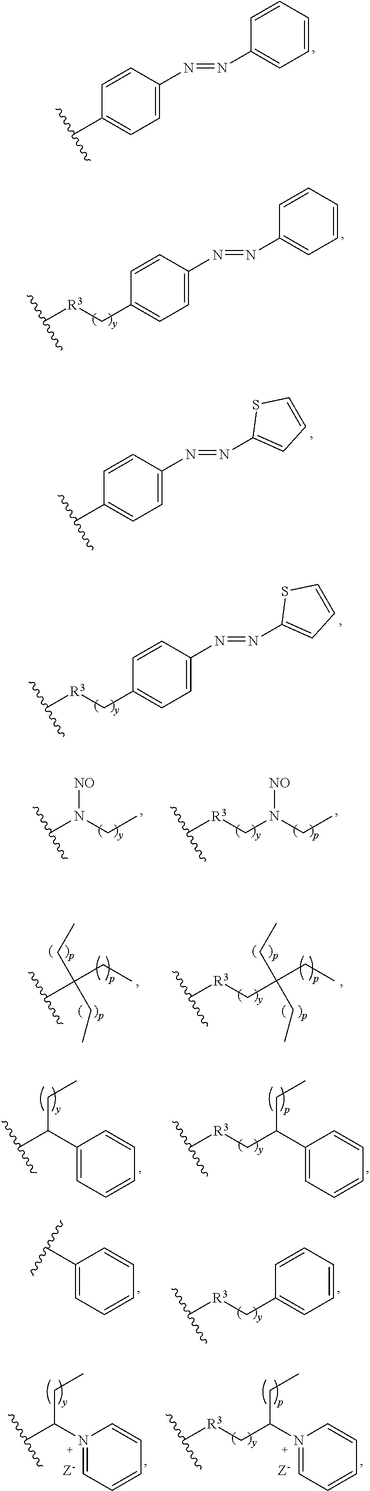 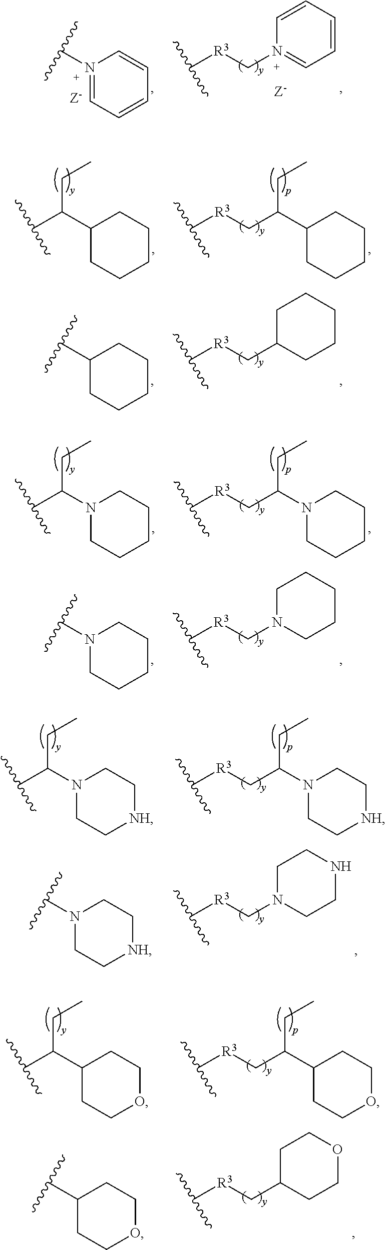 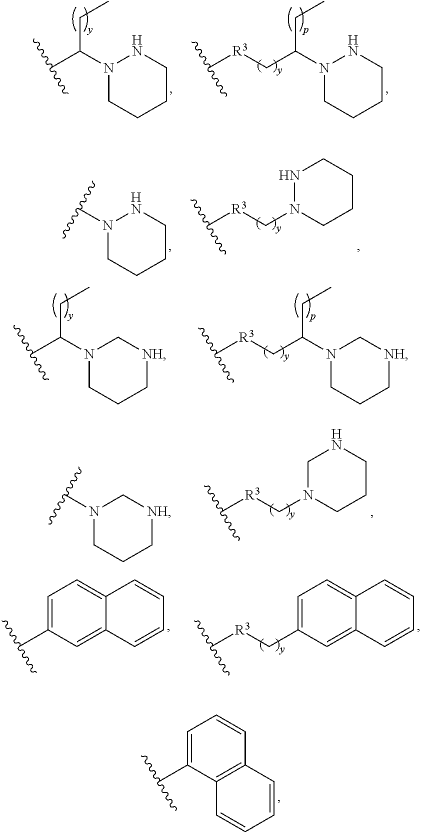 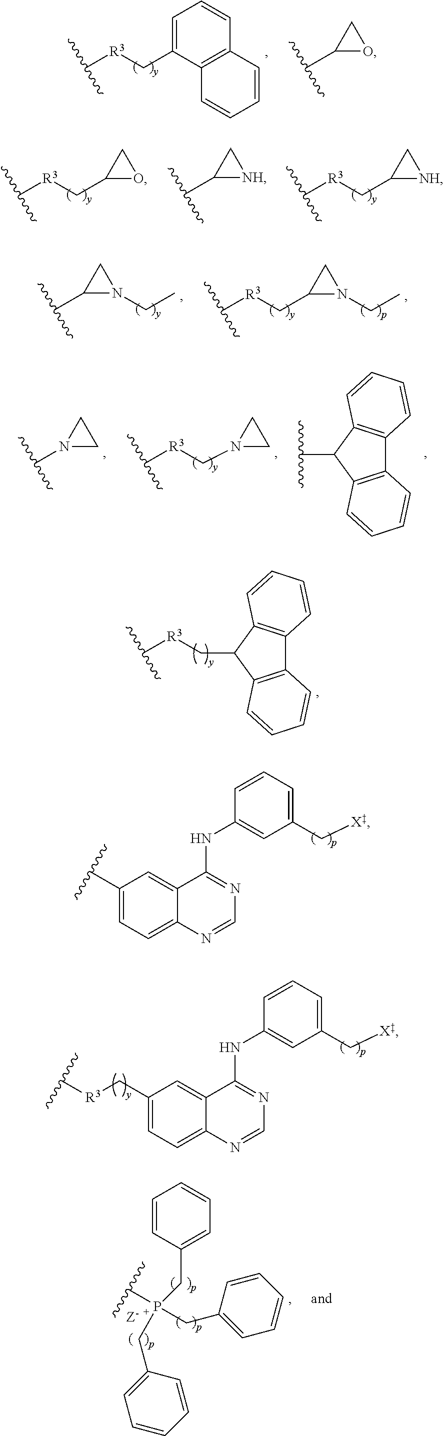 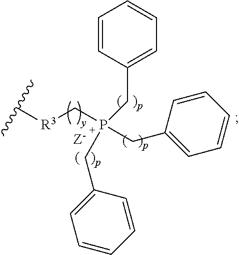
or R⁴ and R⁵ together with the nitrogen to which they are attached may form an optionally substituted 5- to 8-membered saturated or partially unsaturated ring;
or R⁴ and R⁵ function as a linker to a group independently selected from a group defined in R¹;
or R⁴ and R⁵ form a structure together selected from the group considering of: 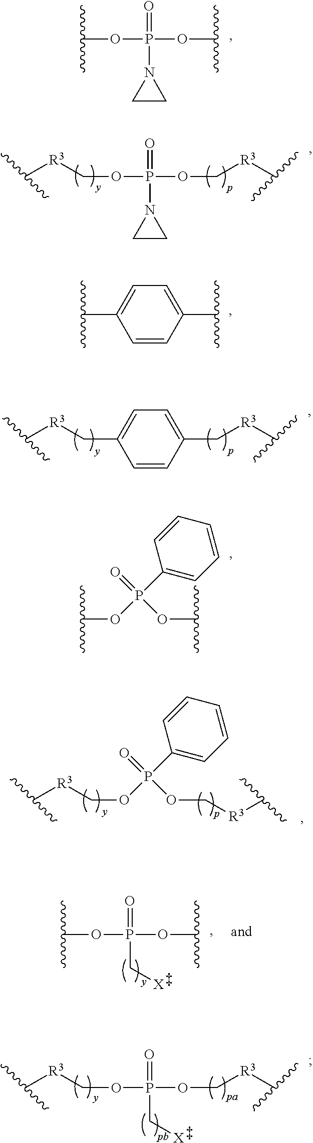
x, q, t, y, s, u, m, n, r, w, y, p, pₐ, and p_{b} are each independently an integer from 0 to 24;
R⁷ is a secondary, tertiary, or reduced amine, an ether, an alcohol;
R⁸ is an alkylated or hydrogenated imide, or an oxygen;
R⁹ is 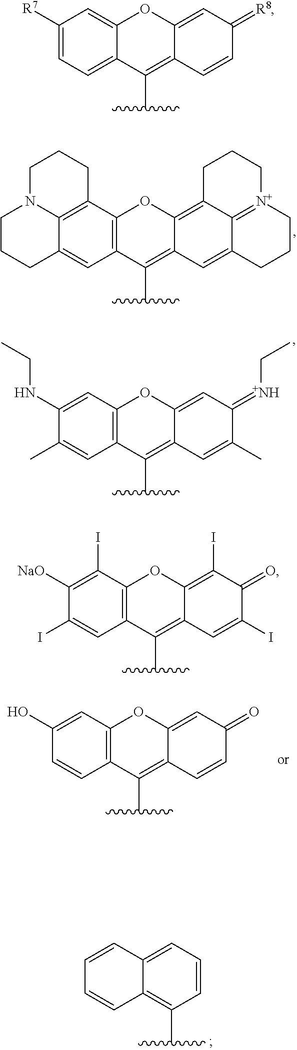
wherein any hydrogen, atom may be substituted with a fluorine, deuterium, and tritium atom;
with the proviso that if the structure is a compound of formula (Ib), then formula (Ib) is substituted with R¹, R² and A;
with the proviso that if X₁ is N and X₂ is C, and R₁ is 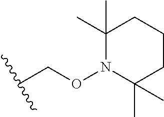 [SMILES: *CON1C(C)(C)CCCC1(C)C]
and one of R² or A is H, then R² or A is not

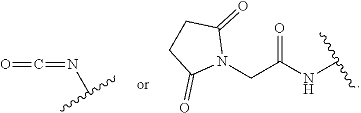

2. The compound of claim 1, which is substituted with R².

3. The compound of claim 1 having formula (Ic)

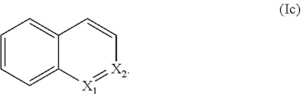

4. The compound of claim 1 having formula (Id), formula (Ie), or formula (If) 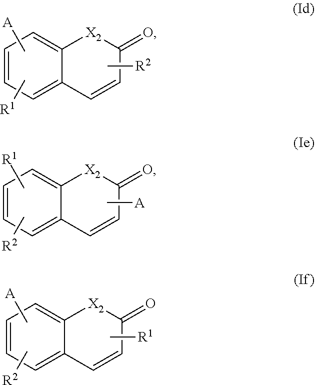
wherein X² is NH, N-alkyl, or NO.

5. The compound of claim 1 which is

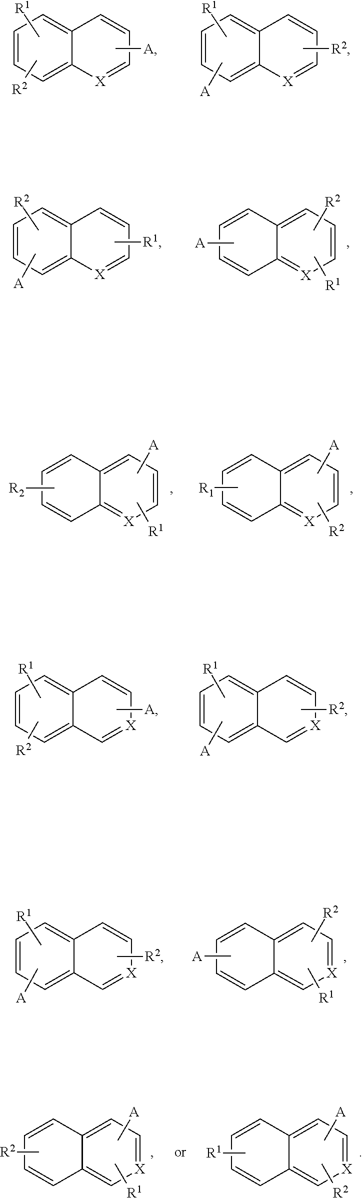

6. The compound of claim 1 which is

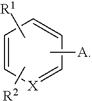

7. The compound of claim 1 which is

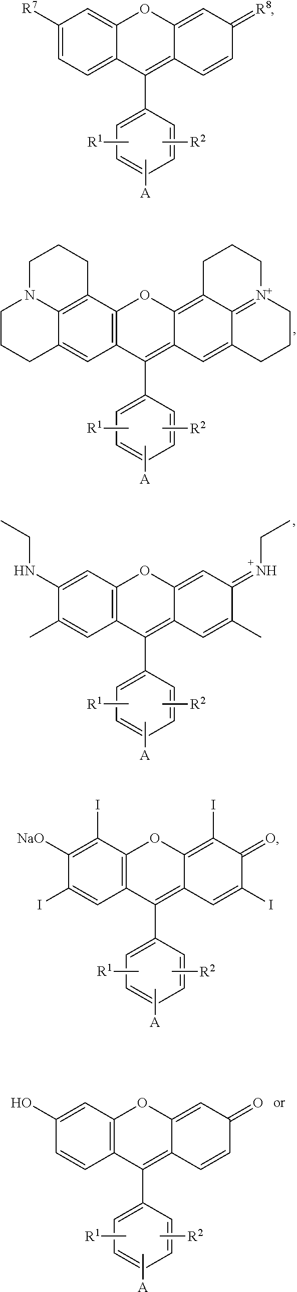

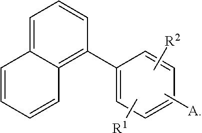

8. A compound which is 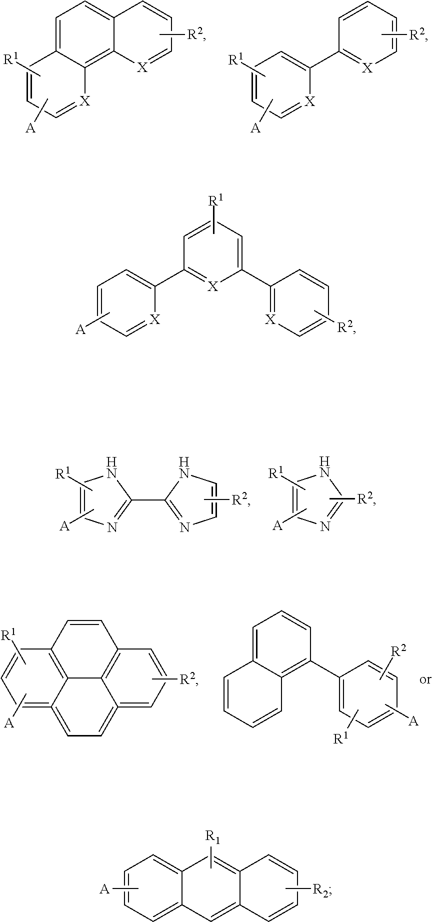
wherein X, A, R¹ and R² are defined as recited in claim 1.

9. The compound of claim 1, wherein R₁ is selected from one of the following structures 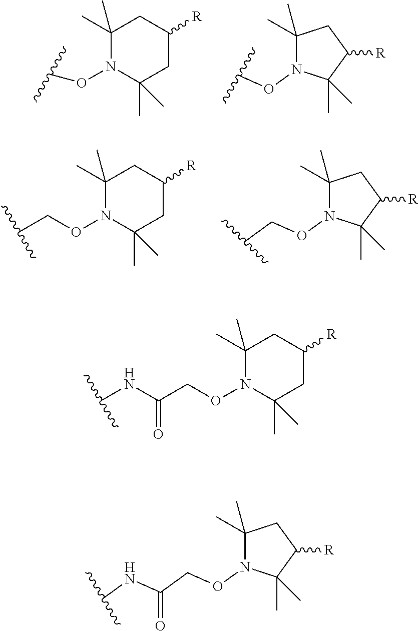
wherein R is independently selected from H, alkyl, alkyl amino, ester, ether, OH, ═O, COOH, NH₂, alkyl phosphonic acid, and halide.

10. The compound of claim 1, wherein R₁ is

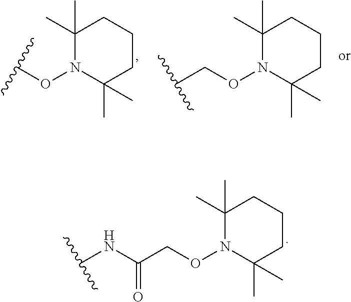

11. The compound of claim 1, wherein R₂ is

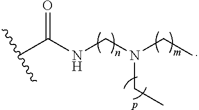

12. The compound of claim 1, wherein R₂ is

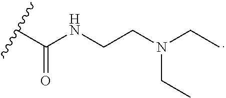 [SMILES: *C(=O)NCCN(CC)CC]

13. The compound of claim 1, wherein A is NH₂,

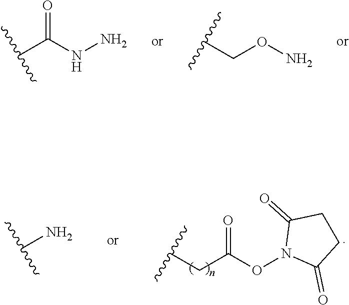

14. The compound of claim 1, wherein A is NH₂,

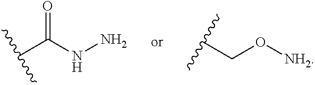

15. The compound of claim 5 wherein X is CH, or N.

16. The compound of claim 1, which is:

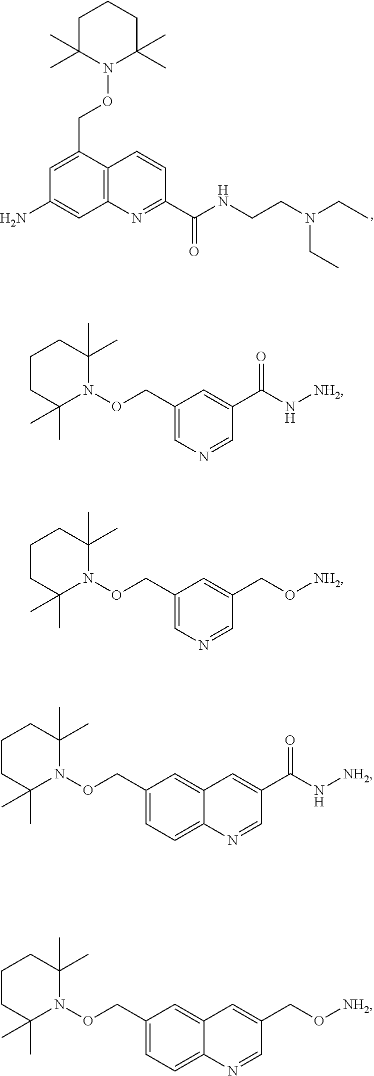 [SMILES: CCN(CC)CCNC(=O)c1ccc2c(CON3C(C)(C)CCCC3(C)C)cc(N)cc2n1 ; CC1(C)CCCC(C)(C)N1OCc1cncc(C(=O)NN)c1 ; CC1(C)CCCC(C)(C)N1OCc1cncc(CON)c1 ; CC1(C)CCCC(C)(C)N1OCc1ccc2ncc(C(=O)NN)cc2c1 ; CC1(C)CCCC(C)(C)N1OCc1ccc2ncc(CON)cc2c1]

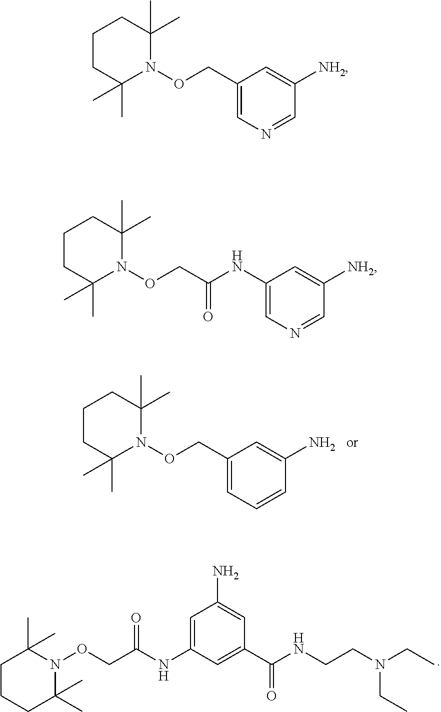 [SMILES: CC1(C)CCCC(C)(C)N1OCc1cncc(N)c1 ; CC1(C)CCCC(C)(C)N1OCC(=O)Nc1cncc(N)c1 ; CC1(C)CCCC(C)(C)N1OCc1cccc(N)c1 ; CCN(CC)CCNC(=O)c1cc(N)cc(NC(=O)CON2C(C)(C)CCCC2(C)C)c1]

17. A method of quantitating and/or characterizing a glycan comprising:
a) obtaining the compound of claim 1;
b) contacting the compound with the glycan, thereby forming a labeled glycan.

18. The method of claim 17, further comprising dissociation of the labeled glycan to form glycan fragmentations.

19. The method of claim 18, wherein the dissociation is collisional-induced dissociation (CID) or higher-energy collision dissociation (HCD).

20. The method of claim 17, further comprising analyzing the labeled glycan or glycan fragmentations with an instrument which is a fluorimeter, a mass spectrometer or liquid chromatography instrument, optionally with an instrument capable of detecting fluorescence and/or absorbance.

21. The method of claim 17, wherein the glycan comprises a reducing terminus.

22. The method of claim 17, wherein the glycan is other than an N-glycan.

23. The method of claim 17 wherein the instrument is a Ultra-Performance Liquid Chromatography (UPLC), a linear quadrupole ion trap (LTQ-XL) mass spectrometer, a Q Exactive Orbitrap mass spectrometer, or a liquid chromatography-mass spectrometry (LC-MS).

24. The method of claim 17 wherein the instrument is equipped with an electrospray ionization (ESI), heated-electrospray ionization (HESI) source and/or with a fluorescence detector.

25. The compound of claim 1 for use in the preparation of a labeled glycan or glycan fragmentations, or for use in glycan detection and/or glycan quantitation.
